(19) United States
(12) Patent Application Publication
(10) Pub. No.: US 2024/0401136 A1
(43) Pub. Date: Dec. 5, 2024

(54) Proprotein Convertase Subtilisin/Kexin Type 1 (PCSK1) Variants And Uses Thereof

(71) Applicant: Regeneron Pharmaceuticals, Inc., Tarrytown, NY (US)

(72) Inventors: Luca Andrea Lotta, Tarrytown, NY (US); Manuel Allen Revez Ferreira, Tarrytown, NY (US)

(21) Appl. No.: 18/746,543

(22) Filed: Jun. 18, 2024

Related U.S. Application Data
(63) Continuation of application No. 17/306,699, filed on May 3, 2021, now Pat. No. 12,049,670.
(60) Provisional application No. 63/019,589, filed on May 4, 2020.

(51) Int. Cl. C12Q 1/6883

(52) U.S. Cl. CPC C12Q 1/6883; C12Q 2600/156

(57) ABSTRACT

Methods of treating obesity and methods of identifying susceptibility of an obese subject to treatment with an agonist of the leptin-melanocortin signaling pathway are provided herein.

### REFERENCE TO A SEQUENCE LISTING

This application includes a Sequence Listing submitted electronically as an XML file named 381204272SEQ, created on Jun. 11, 2024, with a size of 141,694 bytes. The Sequence Listing is incorporated herein by reference.

### FIELD

The present disclosure provides methods of treating subjects having obesity, methods of identifying subjects having an increased risk of developing obesity, and methods of identifying susceptibility of an obese subject to treatment with an agonist of the leptin-melanocortin signaling pathway.

### BACKGROUND

Obesity and its cardio-metabolic complications, in particular type 2 diabetes and coronary artery disease, account for significant morbidity and mortality globally. There is a substantial unmet medical need for safe and effective weight loss approaches.

Lifestyle interventions on diet and physical activity are the first option for the management of obesity and overweight, but efficacy can be limited, and weight regain is common. Bariatric surgery can be highly effective for weight loss in severely obese or high-risk patients, but its use is limited by its invasive nature, cost, risk of perioperative adverse events including perioperative death. While a few drugs have demonstrated efficacy in weight-reduction, pharmacotherapy for the treatment of obesity is limited by the modest weight loss induced by most drugs, side effect profile of some agents, contraindications, low compliance, and barriers to treatment including underprescription.

PCSK1 is a member of the subtilisin-like proprotein convertase family, which includes proteases that process protein and peptide precursors trafficking through regulated or constitutive branches of the secretory pathway. PCSK1 is one of the seven basic amino acid-specific enzymes which cleave their substrates at single or paired basic residues. PCSK1 is packaged into and activated in dense core secretory granules and expressed in the neuroendocrine system and brain. PCSK1 is Involved in the processing of hormone and other protein precursors at sites comprised of pairs of basic amino acid residues. Substrates include proopiomelanocortin (POMC), renin, enkephalin, dynorphin, somatostatin, insulin and AGRP. PCSK1 activity is essential for the activating cleavage of many peptide hormone precursors implicated in the regulation of food ingestion, glucose homeostasis, and energy homeostasis, for example, proopiomelanocortin, proinsulin, proglucagon, and proghrelin.

### SUMMARY

The present disclosure provides methods of treating a subject having obesity comprising administering to the subject an agonist of the leptin-melanocortin signaling pathway.

The present disclosure also provides methods of treating a subject having elevated body mass index (BMI) comprising administering to the subject an agonist of the leptin-melanocortin signaling pathway.

The present disclosure also provides in such methods the detection of the presence in heterozygous state or absence of a PCSK1 variant nucleic acid molecule or PCSK1 variant polypeptide associated with an increased risk of developing obesity and/or elevated BMI in a biological sample from the subject.

The present disclosure also provides methods of identifying susceptibility of an obese subject to treatment with an agonist of the leptin-melanocortin signaling pathway comprising detecting the presence or absence of one copy of a PCSK1 genetic variant associated with an increased risk of developing obesity and/or elevated BMI in a biological sample from the subject, wherein when one copy of a PCSK1 genetic variant is detected, the subject is susceptible to treatment with an agonist of the leptin-melanocortin signaling pathway.

The present disclosure also provides methods of identifying susceptibility of a subject having elevated BMI to treatment with an agonist of the leptin-melanocortin signaling pathway comprising detecting the presence or absence of one copy of a PCSK1 genetic variant associated with an increased risk of developing obesity and/or elevated BMI in a biological sample from the subject, wherein when one copy of a PCSK1 genetic variant is detected, the subject is susceptible to treatment with an agonist of the leptin-melanocortin signaling pathway.

### BRIEF DESCRIPTION OF THE FIGURES

The accompanying figures, which are incorporated in and constitute a part of this specification, illustrate several aspects and together with the description serve to explain some principles of the present disclosure.

The patent or application file contains at least one drawing executed in color. Copies of this patent or patent application publication with color drawings will be provided by the Office upon request and payment of the necessary fee.

FIG. 1 shows the proportion of PCSK1 pLoF carriers among individuals having different categories of BMI from the UK Biobank (UKB) population-based cohort and the MyCode Community Health Initiative cohort from the Geisinger Health System (GHS) healthcare-system-based cohort.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

As used herein, the phrase “corresponding to”, or grammatical variations thereof, when used in the context of the numbering of a particular amino acid or nucleotide sequence or position refers to the numbering of a specified reference sequence when the particular amino acid or nucleotide sequence is compared to the reference sequence (e.g., with the reference sequence herein being the nucleic acid molecule or polypeptide of (wild type) PCSK1). In other words, the residue (e.g., amino acid or nucleotide) number or residue (e.g., amino acid or nucleotide) position of a particular polymer is designated with respect to the reference sequence rather than by the actual numerical position of the residue within the particular amino acid or nucleotide sequence. For example, a particular amino acid sequence or nucleotide sequence can be aligned to a reference sequence by introducing gaps to optimize residue matches between the two sequences. In these cases, although the gaps are present, the numbering of the residue in the particular amino acid or nucleotide sequence is made with respect to the reference sequence to which it has been aligned. A variety of computational algorithms exist that can be used for performing a sequence alignment to identify a nucleotide or amino acid position in one polymeric molecule that corresponds to a nucleotide or amino acid position in another polymeric molecule. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

It has been observed in accordance with the present disclosure that predicted loss of function variants in PCSK1 in the heterozygous state associate with a risk of developing obesity or elevated BMI. Homozygous carrier status for loss of function variants in PCSK1 is a known cause of severe obesity with hormonal disturbances, but the heterozygous carrier state has not been associated with higher BMI or increased risk of obesity before, such that the obese phenotype conferred by LOF variants in PCSK1 is considered an autosomal recessive trait. Because the allele frequency for PCSK1 LOF mutations in European ancestry participants in the UKB and GHS studies is 0.027%, the heterozygous carriers in PCSK1 are over 7000 times more common than homozygous carriers (1 every ˜1,800 people for heterozygous carriers vs 1 every ˜13,500,000 people for homozygous carriers). Hence, the finding of an association between heterozygous carrier state and higher BMI and obesity risk means that the etiologic contribution of PCSK1 LOF variants to obesity is greater than currently thought. Therefore, humans that are heterozygous for PCSK1 alterations that associate with obesity or elevated BMI may be treated such that obesity or elevated BMI is inhibited, the symptoms thereof are reduced, and/or development of symptoms is repressed. In addition, humans that are heterozygous for PCSK1 alterations that associate with obesity or elevated BMI may be treated to prevent obesity by specific interventions, such as any of those disclosed herein. It is also believed that humans having obesity or elevated BMI may be treated with molecules that promote MC4R signaling, which is defective in people with PCSK1 LOF.

For purposes of the present disclosure, any particular human can be categorized as having one of three PCSK1 genotypes: i) PCSK1 reference; ii) heterozygous for a PCSK1 variant nucleic acid molecule associated with an increased risk of developing obesity and/or elevated BMI as discovered herein, and iii) homozygous for a PCSK1 variant nucleic acid molecule associated with an increased risk of developing obesity and/or elevated BMI. A human is PCSK1 reference when the human does not have a copy of a PCSK1 variant nucleic acid molecule associated with an increased risk of developing obesity and/or elevated BMI. A human is heterozygous for a PCSK1 variant nucleic acid molecule associated with an increased risk of developing obesity and/or elevated BMI when the human has a single copy of a PCSK1 variant nucleic acid molecule associated with an increased risk of developing obesity and/or elevated BMI. A subject who has a PCSK1 polypeptide having a partial loss-of-function (or predicted partial loss-of-function) is hypomorphic for PCSK1. A human is homozygous for a PCSK1 variant nucleic acid molecule associated with an increased risk of developing obesity and/or elevated BMI when the human has two copies of any of the PCSK1 variant nucleic acid molecule associated with an increased risk of developing obesity and/or elevated BMI.

For subjects that are genotyped or determined to be heterozygous for a PCSK1 variant nucleic acid molecule associated with an increased risk of developing obesity and/or elevated BMI, such subjects are associated with having increased odds of developing obesity and/or elevated BMI. For subjects that are genotyped or determined to be heterozygous for a PCSK1 variant nucleic acid molecule, such subjects can be treated with an agent effective to treat obesity, such as type 1 obesity, type 2 obesity, or type 3 obesity, and/or with an agent effective to treat increased BMI.

In any of the embodiments described herein, the PCSK1 variant nucleic acid molecule associated with an increased risk of developing obesity and/or elevated BMI can be any PCSK1 nucleic acid molecule (such as, for example, genomic nucleic acid molecule, mRNA molecule, or cDNA molecule produced from the mRNA molecule) encoding a PCSK1 polypeptide having a partial loss-of-function, a complete loss-of-function, a predicted partial loss-of-function, or a predicted complete loss-of-function. In any of the embodiments described herein, the PCSK1 variant nucleic acid molecule associated with an increased risk of developing obesity and/or elevated BMI can also be any PCSK1 variant nucleic acid molecule (such as, for example, genomic nucleic acid molecule, mRNA molecule, or cDNA molecule produced from the mRNA molecule) predicted or shown to be associated with higher BMI, obesity risk, or to result in PCSK1 loss of function in vitro or in vivo. In any of the embodiments described herein, the PCSK1 variant nucleic acid molecule associated with an increased risk of developing obesity and/or elevated BMI can also be any PCSK1 protein truncating variant.

In any of the embodiments described herein, the variant PCSK1 polypeptide can be any PCSK1 polypeptide having a partial loss-of-function, a complete loss-of-function, a predicted partial loss-of-function, or a predicted complete loss-of-function.

The PCSK1 predicted loss-of-function variant nucleic acid molecule can be any one or more of the variant nucleic acid molecules described herein. In some embodiments, the PCSK1 variant nucleic acid molecule associated with an increased risk of developing obesity and/or elevated BMI is a protein truncating variant. A PCSK1 variant nucleic acid molecule associated with an increased risk of developing obesity and/or elevated BMI is also any PCSK1 deleterious missense variant nucleic acid molecule. The PCSK1 deleterious variant nucleic acid molecule can be any one or more of the variant nucleic acid molecules described herein. In some embodiments, the subject has one or two of the following loss-of-function variant PCSK1 nucleic acid molecules: 5:96393172:A:C, 5:96393258:G:A, 5:96393366:G:A, 5:96394941:G:A, 5:96394988:C:T, 5:96397351:CAA:C, 5:96397358:C:T, 5:96397377:C:A, 5:96397382:C:T, 5:96397390:AAC:A, 5:96397440:C:A, 5:96398877:A:G, 5:96398887:GAAGT:G, 5:96398922:A:C, 5:96399952:C:A, 5:96399993:T:A, 5:96400071:G:A, 5:96400106:C:T, 5:96400170:G:A, 5:96408221:A:C, 5:96408288:C:CA, 5:96408299:A:AG, 5:96408310:A:AG, 5:96408324:C:G, 5:96410771:T:TA, 5:96410773:C:T, 5:96410797:C:A, 5:96410840:G:C, 5:96410840:G:T, 5:96410844:CAGGGGGAT:C, 5:96410910:T:TC, 5:96410935:G:GC, 5:96410974:TC:T, 5:96410983:T:TC, 5:96412437:C:CA, 5:96412452:C:A, 5:96412454:A:AT, 5:96416067:G:T, 5:96416072:T:A, 5:96421879:CT:C, 5:96421881:T:A, 5:96421893:TG:T, 5:96421905:G:A, 5:96421940:TA:T, 5:96421951:TG:T, 5:96421954:AT:A, 5:96423354:CA:C, 5:96423401:CAA:C, 5:96425819:C:G, 5:96425827:C:T, 5:96425843:TG:T, 5:96425891:T:A, 5:96425892:AC:A, 5:96429256:CT:C, 5:96429260:G:A, 5:96429269:TG:T, 5:96429310:G:T, 5:96429319:T:C, 5:96432082:C:T, 5:96432861:A:C, 5:96432869:CA:C, 5:96432913:C:A, 5:96432957:CT:C, 5:96432971:AC:A, 5:96432981:G:GCA, 5:96432993:CAA:C, 5:96433040:C:T, 5:96433041:A:G.

In any of the embodiments described herein, the obesity is type 1 obesity, type 2 obesity, or type 3 obesity. In any of the embodiments described herein, the obesity is type 1 obesity. In any of the embodiments described herein, the obesity is type 2 obesity. In any of the embodiments described herein, the obesity is type 3 obesity. In any of the embodiments described herein, the subject has increased BMI.

Symptoms of obesity include, but are not limited to, excess body fat accumulation (particularly around the waist), breathlessness, increased sweating, snoring, inability to cope with sudden physical activity, feeling very tired every day, back and joint pains, skin problems (from moisture accumulating in the folds of skin).

The nucleotide sequence of a PCSK1 reference genomic nucleic acid molecule is set forth in SEQ ID NO:1 (ENST00000311106.8 encompassing chr5:96,390,333-96,433,248 in the GRCh38/hg38 human genome assembly; ENSG00000175426.11), which is 42,916 nucleotides in length. The first nucleotide recited in SEQ ID NO:1 corresponds to the nucleotide at position 96,390,333 of chromosome 5.

The nucleotide sequences of PCSK1 reference mRNA molecules produced through alternative splicing are set forth in SEQ ID NOs:2-12. The variant nucleotides at their respective variant positions for the variant genomic nucleic acid molecules described herein also have corresponding variant nucleotides at their respective variant positions for the variant mRNA molecules based upon the PCSK1 reference mRNA sequences according to SEQ ID NOs:2-12. Any of these PCSK1 variant mRNA molecules can be detected in any of the methods described herein.

The nucleotide sequences of PCSK1 reference cDNA molecules are set forth in SEQ ID NOs:13-23. The variant nucleotides at their respective variant positions for the variant genomic nucleic acid molecules described herein also have corresponding variant nucleotides at their respective variant positions for the variant cDNA molecules based upon the PCSK1 reference cDNA sequence according to SEQ ID NOs:13-23. Any of these PCSK1 variant cDNA molecules can be detected in any of the methods described herein.

The amino acid sequences of PCSK1 reference polypeptide isoforms are set forth in SEQ ID NOs:24-31. Using the translated nucleotide sequence of either the PCSK1 mRNA or cDNA molecules, the PCSK1 variant polypeptides have corresponding translated variant amino acids at variant positions (codons). Any of these PCSK1 variant polypeptides can be detected in any of the methods described herein.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure provides methods of treating a subject having obesity and/or increased BMI comprising administering to the subject an agonist of the leptin-melanocortin signaling pathway. In any of the embodiments described herein, the obesity is type 1 obesity, type 2 obesity, or type 3 obesity.

In any of the embodiments described herein, the agonist of the leptin-melanocortin signaling pathway is an MC4R agonist or a PCSK1 agonist. In some embodiments, the agonist of the leptin-melanocortin signaling pathway is an MC4R agonist. In some embodiments, the agonist of the leptin-melanocortin signaling pathway is a PCSK1 agonist. In some embodiments, the leptin-melanocortin signaling pathway agonist comprises a protein, a peptide, a nucleic acid molecule, or a small molecule.

In some embodiments, the MC4R agonist is a protein, such as recombinant MC4R. In some embodiments, the MC4R agonist is a peptide. In some embodiments, the peptide MC4R agonist is setmelanotide. In some embodiments, the peptide MC4R agonist is a peptide having the amino acid sequence His-Phe-Arg-Trp (SEQ ID NO:32). In some embodiments, the MC4R agonist is a peptide mimetic, such as (1,2,3R,4-tetrahydroisoquinoline-3-carboxylic acid). In some embodiments, the MC4R agonist is a small molecule, such as ALB-127158(a).

In some embodiments, the PCSK1 agonist is a protein, such as recombinant PCSK1. In some embodiments, the PCSK1 agonist is a peptide analog of PCSK1. In some embodiments, the PCSK1 agonist is a peptide.

In any of the embodiments described herein, the subject having obesity and/or elevated BMI can be treated with additional therapeutic agents. Examples of therapeutic agents that treat or inhibit obesity include, but are not limited to, sibutramine, orlistat, phentermine plus topiramate, lorcaserin, bupropion plus naltrexone, liraglutide, phentermine plus diethylpropion, bupropion, metformin, pramlintide, topiramate, and zonisamide, or any combination thereof. In some embodiments, the therapeutic agent that treats or inhibits obesity and/or reduces BMI is a combination of setmelanotide and one or more of sibutramine, orlistat, phentermine, lorcaserin, naltrexone, liraglutide, diethylpropion, bupropion, metformin, pramlintide, topiramate, and zonisamide.

Administration of the therapeutic agents that treat or inhibit obesity or elevated BMI can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit obesity or elevated BMI can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in obesity, decreased BMI, or both, a decrease/reduction in the severity of obesity, a decrease/reduction in symptoms and obesity-related effects, elevated BMI-related effects, or both, delaying the onset of symptoms and obesity-related effects, elevated BMI-related effects, or both, reducing the severity of symptoms of obesity-related effects, or of elevated BMI-related effects, or both, reducing the number of symptoms and obesity-related effects, or of elevated BMI-related effects, or both, reducing the latency of symptoms and obesity-related effects, or of elevated BMI-related effects, or both, an amelioration of symptoms and obesity-related effects, or of elevated BMI-related effects, or both, reducing secondary symptoms, reducing secondary infections, preventing relapse to obesity, or to elevated BMI, or both, decreasing the number or frequency of relapse episodes, increasing latency between symptomatic episodes, increasing time to sustained progression, speeding recovery, and/or increasing efficacy of or decreasing resistance to alternative therapeutics, following administration of the agent or composition comprising the agent. A prophylactic effect may comprise a complete or partial avoidance/inhibition or a delay of obesity, or of elevated BMI, or both development/progression (such as, for example, a complete or partial avoidance/inhibition or a delay) following administration of a therapeutic protocol. Treatment of obesity, elevated BMI, or both encompasses the treatment of subjects already diagnosed as having any form of obesity, or of elevated BMI, or both at any clinical stage or manifestation, the delay of the onset or evolution or aggravation or deterioration of the symptoms or signs of obesity, or of elevated BMI, or both, and/or preventing and/or reducing the severity of obesity, or of elevated BMI, or both.

In any of the embodiments described herein, the methods can further comprise detecting the presence or absence of a PCSK1 variant nucleic acid molecule or variant polypeptide associated with an increased risk of developing obesity and/or elevated BMI in a biological sample from the subject. It is understood that gene sequences within a population and mRNA molecules encoded by such genes can vary due to polymorphisms such as single-nucleotide polymorphisms. The sequences provided herein for the PCSK1 variant nucleic acid molecules disclosed herein are only exemplary sequences. Other sequences for the PCSK1 variant nucleic acid molecules are also possible. The PCSK1 variant nucleic acid molecule or variant polypeptide associated with an increased risk of developing obesity and/or elevated BMI can be any PCSK1 loss-of-function variant or PCSK1 missense variant, such as any of those described herein.

In some embodiments, detecting the presence or absence of the PCSK1 variant nucleic acid molecule or variant polypeptide associated with an increased risk of developing obesity and/or elevated BMI comprises determining whether the subject has a PCSK1 variant genomic nucleic acid molecule associated with an increased risk of developing obesity and/or elevated BMI, a PCSK1 variant mRNA molecule associated with an increased risk of developing obesity and/or elevated BMI, a PCSK1 variant cDNA molecule produced from the mRNA molecule, and/or a PCSK1 variant polypeptide associated with an increased risk of developing obesity and/or elevated BMI. In some embodiments, such determination is carried out by obtaining or having obtained a biological sample from the subject, and performing or having performed an assay on the biological sample to determine whether the subject has a PCSK1 variant nucleic acid molecule or variant polypeptide associated with an increased risk of developing obesity and/or elevated BMI.

Determining whether a subject has a PCSK1 variant nucleic acid molecule or a PCSK1 variant polypeptide associated with an increased risk of developing obesity and/or elevated BMI in a biological sample can be carried out by any of the methods described herein. In some embodiments, the detecting step, determining step, or assay is carried out in vitro. In some embodiments, these methods can be carried out in situ. In some embodiments, these methods can be carried out in vivo. In any of these embodiments, the nucleic acid molecule can be present within a cell obtained from the subject. In some embodiments, the assay is a sequence analysis or genotyping assay for nucleic acid molecules. In some embodiments, the assay is an immunoassay for polypeptides.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. In some embodiments, the biological sample comprises a cell lysate. Such methods can further comprise obtaining a biological sample from the subject. The sample used in the methods disclosed herein will vary based on the assay format, nature of the detection method, and the tissues, cells, or extracts that are used as the sample. A biological sample can be processed differently depending on the assay being employed. For example, when detecting any PCSK1 variant nucleic acid molecule, preliminary processing designed to isolate or enrich the sample for the genomic DNA can be employed. A variety of known techniques may be used for this purpose. When detecting the level of any PCSK1 variant mRNA, different techniques can be used enrich the biological sample with mRNA. Various methods to detect the presence or level of a mRNA or the presence of a particular variant genomic DNA locus can be used.

In some embodiments, the methods can further comprise determining the subject's aggregate burden of having one copy of a PCSK1 variant genomic nucleic acid molecule associated with an increased risk of developing obesity and/or elevated BMI, PCSK1 variant mRNA molecules associated with an increased risk of developing obesity and/or elevated BMI, PCSK1 variant cDNA molecules produced from the mRNA molecules, and/or PCSK1 variant polypeptides associated with an increased risk of developing obesity and/or elevated BMI. The aggregate burden is the sum of all rare variants in the heterozygous state for the PCSK1 gene, which can be carried out in an association analysis with obesity. In some embodiments, the subject is heterozygous for one copy of any of the PCSK1 variants associated with an increased risk of developing obesity and/or elevated BMI. The result of the association analysis suggests that a heterozygous state of rare loss-of-function and missense variants of PCSK1 are associated with increased risk of obesity.

In some embodiments, the detecting step, determining step, or assay comprises sequencing at least a portion of the nucleotide sequence of the PCSK1 nucleic acid molecule in the biological sample. The sequenced portion comprises a position corresponding to a loss-of-function variant position. When a variant nucleotide at the loss-of-function variant position is detected, the PCSK1 nucleic acid molecule in the biological sample is a PCSK1 loss-of-function variant nucleic acid molecule. The loss-of-function variant position within any particular PCSK1 nucleic acid molecule is the one or more positions of the variant nucleotide sequence that are different compared to the nucleotide sequence of the corresponding reference nucleic acid molecule.

In some embodiments, the detecting step, determining step, or assay comprises: contacting the biological sample with a primer hybridizing to a portion of the nucleotide sequence of the PCSK1 nucleic acid molecule that is proximate to a loss-of-function variant position, extending the primer at least through the loss-of-function variant position, and determining whether the extension product of the primer comprises a variant nucleotide at the loss-of-function variant position. In some embodiments, the detecting step, determining step, or assay comprises sequencing the entire nucleic acid molecule.

In some embodiments, the detecting step, determining step, or assay comprises sequencing at least a portion of the nucleotide sequence of the PCSK1 nucleic acid molecule in the biological sample. The sequenced portion comprises a position corresponding to a missense variant position. When a variant nucleotide at the missense variant position is detected, the PCSK1 nucleic acid molecule in the biological sample is a PCSK1 missense variant nucleic acid molecule. The missense variant position within any particular PCSK1 nucleic acid molecule is the one or more positions of the variant nucleotide sequence that are different compared to the nucleotide sequence of the corresponding reference nucleic acid molecule.

In some embodiments, the detecting step, determining step, or assay comprises contacting the biological sample with a primer hybridizing to a portion of the nucleotide sequence of the PCSK1 nucleic acid molecule that is proximate to a missense variant position, extending the primer at least through the missense variant position, and determining whether the extension product of the primer comprises a variant nucleotide at the missense variant position. In some embodiments, the detecting step, determining step, or assay comprises sequencing the entire nucleic acid molecule.

In some embodiments, the assay comprises contacting the biological sample with a primer, such as an alteration-specific primer, that specifically hybridizes to a PCSK1 variant genomic sequence, variant mRNA sequence, or variant cDNA sequence and not the corresponding PCSK1 reference sequence under stringent conditions.

In some embodiments, only a PCSK1 genomic nucleic acid molecule is analyzed. In some embodiments, only a PCSK1 mRNA is analyzed. In some embodiments, only a PCSK1 cDNA obtained from PCSK1 mRNA is analyzed.

Illustrative examples of nucleic acid sequencing techniques include, but are not limited to, chain terminator (Sanger) sequencing and dye terminator sequencing. In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the detecting step, determining step, or assay comprises amplifying at least a portion of the PCSK1 nucleic acid molecule, wherein the portion comprises a loss-of-function variant position, labeling the amplified nucleic acid molecule with a detectable label, contacting the labeled nucleic acid molecule with a support comprising an alteration-specific probe, wherein the alteration-specific probe comprises a nucleotide sequence which hybridizes under stringent conditions to the loss-of-function variant position, and detecting the detectable label. In some embodiments, the nucleic acid molecule in the sample is mRNA and the mRNA is reverse-transcribed into a cDNA prior to the amplifying step.

In some embodiments, the detecting step, determining step, or assay comprises contacting the nucleic acid molecule in the biological sample with an alteration-specific probe comprising a detectable label, wherein the alteration-specific probe comprises a nucleotide sequence which hybridizes under stringent conditions to a loss-of-function variant position, and detecting the detectable label.

In some embodiments, the detecting step, determining step, or assay comprises amplifying at least a portion of the PCSK1 nucleic acid molecule, wherein the portion comprises a missense variant position, labeling the amplified nucleic acid molecule with a detectable label, contacting the labeled nucleic acid molecule with a support comprising an alteration-specific probe, wherein the alteration-specific probe comprises a nucleotide sequence which hybridizes under stringent conditions to the missense variant position, and detecting the detectable label. In some embodiments, the nucleic acid molecule in the sample is mRNA and the mRNA is reverse-transcribed into a cDNA prior to the amplifying step.

In some embodiments, the detecting step, determining step, or assay comprises contacting the nucleic acid molecule in the biological sample with an alteration-specific probe comprising a detectable label, wherein the alteration-specific probe comprises a nucleotide sequence which hybridizes under stringent conditions to a missense variant position, and detecting the detectable label.

The alteration-specific probes or alteration-specific primers described herein comprise a nucleic acid sequence which is complementary to and/or hybridizes, or specifically hybridizes, to a PCSK1 loss-of-function variant nucleic acid molecule, or a PCSK1 missense variant nucleic acid molecule, or the complement thereof. In some embodiments, the alteration-specific probes or alteration-specific primers comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 nucleotides. In some embodiments, the alteration-specific probes or alteration-specific primers comprise or consist of at least 15 nucleotides. In some embodiments, the alteration-specific probes or alteration-specific primers comprise or consist of at least 15 nucleotides to at least about 35 nucleotides. In some embodiments, alteration-specific probes or alteration-specific primers hybridize to PCSK1 loss-of-function variant genomic nucleic acid molecules, PCSK1 loss-of-function variant mRNA molecules, and/or PCSK1 loss-of-function variant cDNA molecules under stringent conditions. In some embodiments, alteration-specific probes or alteration-specific primers hybridize to PCSK1 missense variant genomic nucleic acid molecules, PCSK1 missense variant mRNA molecules, and/or PCSK1 missense variant cDNA molecules under stringent conditions.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the assay comprises contacting the biological sample with a probe, such as an alteration-specific probe, that specifically hybridizes to a PCSK1 variant genomic sequence, variant mRNA sequence, or variant cDNA sequence and not the corresponding PCSK1 reference sequence under stringent conditions, and determining whether hybridization has occurred.

In some embodiments, the methods utilize probes and primers of sufficient nucleotide length to bind to the target nucleotide sequence and specifically detect and/or identify a polynucleotide comprising a PCSK1 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, labeled primers or probes directed against purified DNA, amplified DNA, and fixed cell preparations (fluorescence in situ hybridization (FISH)) can be used for detection. In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence-based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. Stringent conditions are sequence-dependent and will be different in different circumstances.

Appropriate stringency conditions which promote DNA hybridization, for example, 6×sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, detecting the presence of a human PCSK1 variant polypeptide comprises performing an assay on a sample obtained from a subject to determine whether a PCSK1 polypeptide in the subject contains one copy of a variation that causes the polypeptide to have a loss-of-function (partial or complete) or predicted loss-of-function (partial or complete), or be produced from a missense variant nucleic acid molecule, or be a truncated variant polypeptide. In some embodiments, the assay comprises sequencing at least a portion of the PCSK1 polypeptide that comprises a variant position. In some embodiments, the detecting step comprises sequencing the entire polypeptide. Identification of a variant amino acid at the variant position of the PCSK1 polypeptide indicates that the PCSK1 polypeptide is a PCSK1 loss-of-function polypeptide, or is produced from a missense variant nucleic acid molecule. In some embodiments, the assay comprises an immunoassay for detecting the presence of a variant polypeptide. Detection of a variant amino acid at the variant position of the PCSK1 polypeptide indicates that the PCSK1 polypeptide is a variant PCSK1 polypeptide.

The probes and/or primers (including alteration-specific probes and alteration-specific primers) described herein comprise or consist of from about 15 to about 100, from about 15 to about 35 nucleotides. In some embodiments, the alteration-specific probes and alteration-specific primers comprise DNA. In some embodiments, the alteration-specific probes and alteration-specific primers comprise RNA. In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers (including alteration-specific probes and alteration-specific primers) specifically hybridize to any of the nucleic acid molecules disclosed herein under stringent conditions. In the context of the disclosure “specifically hybridizes” means that the probe or primer (including alteration-specific probes and alteration-specific primers) does not hybridize to a nucleic acid sequence encoding a PCSK1 reference genomic nucleic acid molecule, a PCSK1 reference mRNA molecule, and/or a PCSK1 reference cDNA molecule. In some embodiments, the probes (such as, for example, an alteration-specific probe) comprise a label. In some embodiments, the label is a fluorescent label, a radiolabel, or biotin.

The present disclosure also provides methods of identifying a subject having an increased risk of developing obesity. The methods comprise determining or having determined whether the subject has any one copy of the PCSK1 loss-of-function variant nucleic acid molecule or polypeptide produced therefrom described herein. The methods can also comprise determining or having determined whether the subject has any one copy of the PCSK1 missense variant nucleic acid molecule or polypeptide produced therefrom described herein. When the subject has one copy of a PCSK1 loss-of-function variant nucleic acid molecule, or a PCSK1 missense variant nucleic acid molecule, or polypeptide produced therefrom, the subject has an increased risk of developing obesity. In some embodiments, the subject is heterozygous for a PCSK1 loss-of-function variant or PCSK1 missense variant. In some embodiments, when a subject is identified as having an increased risk of developing obesity (such as being heterozygous for a PCSK1 loss-of-function variant or PCSK1 missense variant), the subject is further treated with a therapeutic agent that treats or inhibits obesity and/or a leptin-melanocortin signaling pathway agonist (for example, an MC4R agonist), as described herein. Any of the leptin-melanocortin signaling pathway agonists described herein can be administered to the subject.

The present disclosure also provides methods of identifying a subject having an increased risk of developing elevated BMI. The methods comprise determining or having determined whether the subject has any one copy of the PCSK1 loss-of-function variant nucleic acid molecule or polypeptide produced therefrom described herein. The methods can also comprise determining or having determined whether the subject has any one copy of the PCSK1 missense variant nucleic acid molecule or polypeptide produced therefrom described herein. When the subject has one copy of a PCSK1 loss-of-function variant nucleic acid molecule, or a PCSK1 missense variant nucleic acid molecule, or polypeptide produced therefrom, the subject has an increased risk of developing elevated BMI. In some embodiments, the subject is heterozygous for a PCSK1 loss-of-function variant or PCSK1 missense variant. In some embodiments, when a subject is identified as having an increased risk of developing elevated BMI (such as being heterozygous for a PCSK1 loss-of-function variant or PCSK1 missense variant), the subject is further treated with a therapeutic agent that treats or inhibits elevated BMI and/or a leptin-melanocortin signaling pathway agonist (for example, an MC4R agonist), as described herein. Any of the leptin-melanocortin signaling pathway agonists described herein can be administered to the subject.

The present disclosure also provides methods of diagnosing obesity in a subject. The methods comprise determining or having determined whether the subject has any one copy of the PCSK1 loss-of-function variant nucleic acid molecule or polypeptide produced therefrom described herein. The methods can also comprise determining or having determined whether the subject has any one copy of the PCSK1 missense variant nucleic acid molecule or polypeptide produced therefrom described herein. When the subject has one copy of a PCSK1 loss-of-function variant nucleic acid molecule, or a PCSK1 missense variant nucleic acid molecule, or polypeptide produced therefrom, and has one or more symptoms of obesity, the subject is diagnosed as having obesity. In some embodiments, the subject is heterozygous for a PCSK1 loss-of-function variant or PCSK1 missense variant. In some embodiments, when a subject is identified as having obesity (such as having one or more symptoms of obesity and being heterozygous for a PCSK1 loss-of-function variant or PCSK1 missense variant), the subject is further treated with a therapeutic agent that treats or inhibits obesity and/or a leptin-melanocortin signaling pathway agonist (for example, an MC4R agonist), as described herein. Any of the leptin-melanocortin signaling pathway agonists described herein can be administered to the subject.

The present disclosure also provides methods of diagnosing elevated BMI in a subject. The methods comprise determining or having determined whether the subject has any one copy of a PCSK1 loss-of-function variant nucleic acid molecule or polypeptide produced therefrom described herein. The methods can also comprise determining or having determined whether the subject has any one copy of a PCSK1 missense variant nucleic acid molecule or polypeptide produced therefrom described herein. When the subject has one copy of a PCSK1 loss-of-function variant nucleic acid molecule, or a PCSK1 missense variant nucleic acid molecule, or polypeptide produced therefrom, and has one or more symptoms of obesity, the subject is diagnosed as having elevated BMI. In some embodiments, the subject is heterozygous for PCSK1 loss-of-function variant or PCSK1 missense variant. In some embodiments, when a subject is identified as having elevated BMI (such as having one or more symptoms of obesity and being heterozygous for a PCSK1 loss-of-function variant or PCSK1 missense variant), the subject is further treated with a therapeutic agent that treats or inhibits elevated BMI and/or a leptin-melanocortin signaling pathway agonist (for example, an MC4R agonist), as described herein. Any of the leptin-melanocortin signaling pathway agonists described herein can be administered to the subject.

The present disclosure also provides methods of identifying susceptibility of obese subjects to treatment with a leptin-melanocortin signaling pathway agonist (for example, an MC4R agonist). The methods comprise determining or having determined whether the subject has any one copy of a PCSK1 loss-of-function variant nucleic acid molecules or polypeptides produced therefrom as described herein. The methods can also comprise determining or having determined whether the subject has any one copy of a PCSK1 missense variant nucleic acid molecules or polypeptides produced therefrom as described herein. When the subject is heterozygous for a PCSK1 loss-of-function variant nucleic acid molecule, or a PCSK1 missense variant nucleic acid molecule, or a polypeptide produced therefrom, the subject is susceptible to treatment with a leptin-melanocortin signaling pathway agonist (for example, an MC4R agonist). In some embodiments, when a subject is identified as susceptible to treatment (such as being heterozygous for a PCSK1 loss-of-function variant or PCSK1 missense variant), the subject is further treated with a leptin-melanocortin signaling pathway agonist (for example, an MC4R agonist), as described herein. Any of the leptin-melanocortin signaling pathway agonists described herein can be administered to the subject.

The present disclosure also provides methods of identifying susceptibility of subjects having elevated BMI to treatment with a leptin-melanocortin signaling pathway agonist (for example, an MC4R agonist). The methods comprise determining or having determined whether the subject has any one copy of a PCSK1 loss-of-function variant nucleic acid molecule or polypeptide produced therefrom described herein. The methods can also comprise determining or having determined whether the subject has any one copy of a PCSK1 missense variant nucleic acid molecule or polypeptide produced therefrom described herein. When the subject is heterozygous for a PCSK1 loss-of-function variant nucleic acid molecule, or a PCSK1 missense variant nucleic acid molecule, or a polypeptide produced therefrom, the subject is susceptible to treatment with a leptin-melanocortin signaling pathway agonist (for example, an MC4R agonist). In some embodiments, when a subject is identified as susceptible to treatment (such as being heterozygous for a PCSK1 loss of function variant or PCSK1 missense variant), the subject is further treated with a leptin-melanocortin signaling pathway agonist (for example, an MC4R agonist), as described herein. Any of the leptin-melanocortin signaling pathway agonists described herein can be administered to the subject.

The present disclosure also provides molecular complexes comprising any of the PCSK1 variant nucleic acid molecules (genomic nucleic acid molecules, mRNA molecules, or cDNA molecules), or complement thereof, described herein and any of the alteration-specific primers or alteration-specific probes described herein. In some embodiments, the PCSK1 variant nucleic acid molecules (genomic nucleic acid molecules, mRNA molecules, or cDNA molecules), or complement thereof, in the molecular complexes are single-stranded. In some embodiments, the PCSK1 variant nucleic acid molecule is any of the variant genomic nucleic acid molecules described herein. In some embodiments, the PCSK1 variant nucleic acid molecule is any of the variant mRNA molecules described herein. In some embodiments, the PCSK1 variant nucleic acid molecule is any of the variant cDNA molecules described herein. In some embodiments, the molecular complex comprises any of the PCSK1 variant nucleic acid molecules (genomic nucleic acid molecules, mRNA molecules, or cDNA molecules), or complement thereof, described herein and any of the alteration-specific primers described herein. In some embodiments, the molecular complex comprises any of the PCSK1 variant nucleic acid molecules (genomic nucleic acid molecules, mRNA molecules, or cDNA molecules), or complement thereof, described herein and any of the alteration-specific probes described herein. In some embodiments, the molecular complex comprises a non-human polymerase.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Aggregate Burden

### Experimental Design

Overview: Genetic association studies were performed in the United Kingdom (UK) Biobank (UKB) cohort (Sudlow et al., PLOS Med., 2015, 12, e1001779) and in the MyCode Community Health Initiative cohort from the Geisinger Health System (GHS) (Carey et al., Genet. Med., 2016, 18, 906-913). The UKB is a population-based cohort study of people aged between 40 and 69 from 22 testing centers in the UK. Recruitment took place between 2006 and 2010 (Sudlow et al., PLOS Med., 2015, 12, e1001779). A total of 142,720 European ancestry participants with available whole-exome sequencing and clinical phenotype data were included in this study. The GHS MyCode study is a hospital-based cohort of patients of the GHS (Carey et al., Genet. Med., 2016, 18, 906-913). Recruitment took place between 2007 and 2019. A total of 132,634 European ancestry participants with available whole-exome sequencing and clinical phenotype data were included in this study.

Definition of BMI (UKB and GHS Studies): In both UKB and GHS, body mass index (BMI) was calculated as weight in kilograms divided by height in meters squared on the basis of anthropometric measurements taken at one of the study visits. BMI categories were defined on the basis of the World Health Organization classification.

### Sample Preparation and Sequencing

A high-throughput automated approach was used to prepare 100 ng of high-quality genomic DNA for exome capture. The exome was captured using NimbleGen probes (SeqCap VCRome) for a subset of the GHS study (called GHS-VCRome herein) or a slightly modified version of the xGen design available from Integrated DNA Technologies (IDT) for a second subset of GHS (called GHS-IDT herein) or UKB.

A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. The captured DNA was amplified by polymerase chain reaction. The multiplexed samples were sequenced using 75 bp paired-end sequencing on an Illumina v4 HiSeq 2500 (GHS-VCRome subset and part of the GHS-IDT subset) or NovaSeq (part of the GHS-IDT subset and UKB). Sequencing had a coverage depth (i.e., number of sequence-reads covering each nucleotide in the target areas of the genome) sufficient to provide greater than 20x coverage over 85% of targeted bases in 96% of VCRome samples and 20X coverage over 90% of targeted bases in 99% of IDT samples.

Sequence Alignment: Raw sequence data from the Illumina sequencers were uploaded to the DNAnexus platform. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations), and single nucleotide variants and intra-read insertion-deletion calling with genotyping software. Sequencing and data quality metric statistics were captured for each sample to evaluate capture, alignment, and variant calling performance.

Sample-and project-level variant calls files (VCFs) were generated. Project-level VCFs contained genotype and the associated metric information for all samples at any site where any sample in the cohort carried a variant from the reference genome.

Variant Annotation and Definition of LOF and Predicted Deleterious Genetic Variants: Single nucleotide variants and insertion-deletions were annotated with the snpEff software using the Ensembl v85 gene definitions to determine their functional impact on transcripts and genes. The functional annotations were then further processed to get a single effect prediction (Regeneron Effect Prediction; REP) for each variant.

To reduce the number of false-positive LOF genetic variant calls, restrictions were imposed on the transcripts in the annotation set. A subset of 54,214 protein-coding transcripts with annotated start and stop transcription sites were considered out of the total 198,002 transcripts in Ensembl v85.

The snpEff predictions that involve protein-coding transcripts with an annotated start and stop were then combined into a single functional impact prediction (i.e., REP) by selecting the most deleterious functional effect class for each gene. The hierarchy used to determine the deleteriousness of a functional effect class is set forth in Table 1.

LOF genetic variants were defined as variants that are predicted to disrupt gene function, by resulting in the likely loss of a copy of the functional gene product. These included insertions or deletions resulting in a frameshift, insertions or deletions or single nucleotide variants resulting in the introduction of a premature stop codon or in the loss of the transcription start site or stop site of the transcript, and variants in donor or acceptor splice sites (see, Table 1 for a full list of variant definitions and effect priority).

TABLE 1
Variant Definitions and Effect Priority

| | | | Is a |
|---|---|---|---|
| | | Effect | Predicted |
| Effect | Description | Priority | LOF Variant |
| Frameshift | Variant causes a frameshift | 1 | Yes |
| | (eg, insertion or deletion | | |
| | (Indel) size that is not a | | |
| | multiple of 3) | | |
| Stop_gained | Variant causes a STOP codon | 2 | Yes |
| | (eg, Cag/Tag, Q/*) | | |
| Start_lost | Variant causes start codon to | 3 | Yes |
| | be mutated into a non-start | | |
| | codon (eg, aTg/aGg, M/R) | | |
| Splice_acceptor | Variant hits a splice acceptor | 4 | Yes |
| | site (defined as 2 bases | | |
| | before exon start, except for | | |
| | the first exon) | | |
| Splice_donor | Variant hits a splice donor | 5 | Yes |
| | site (defined as 2 bases after | | |
| | coding exon end, except | | |
| | for the last exon) | | |
| Stop_lost | Variant causes stop codon to | 6 | Yes |
| | be mutated into a non-stop | | |
| | codon (eg, Tga/Cga, */R) | | |
| Inframe_indel | Variant inserts or deletes 1 or | 7 | No |
| | many codons (ie, a multiple | | |
| | of 3) | | |
| Missense | Variant causes a codon that | 8 | No |
| | produces a different amino | | |
| | acid (eg, Tgg/Cgg, W/R) | | |
| Splice_region | Variant occurs within the | 9 | No |
| | region of the splice site, either | | |
| | within 1-3 bases of the exon | | |
| | or 3-8 bases of the intron | | |
| Synonymous | Variant causes a codon that | 10 | No |
| | produces the same amino acid | | |
| | (eg, Ttg/Ctg, L/L) | | |
| Regulatory_region | Variant hits a known | 11 | No |
| | regulatory feature | | |
| | (non-coding) | | |
| 5_prime_UTR | Variant hits 5′UTR region | 12 | No |
| 3_prime_UTR | Variant hits 3′UTR region | 13 | No |
| Upstream | Variant is upstream of a gene | 14 | No |
| | (default length: 5K bases) | | |
| Downstream | Variant is downstream of a | 15 | No |
| | gene (default length: 5K | | |
| | bases) | | |
| Intronic | Variant hits and intron | 16 | No |
| Intragenic | Variant hits a gene, but no | 17 | No |
| | transcripts within the gene | | |

Missense variants were classified into three groups based on their predicted effect on the PCSK1 protein: predicted to be deleterious by (1) 5/5 in silico prediction algorithms (M3 missense variants), (2) 1/5 algorithms (M4 missense variants), or (3) 0/5 algorithms (M2 missense variants). The prediction algorithms used were: 1) SIFT (available at the world wide web at “ncbi.nlm.nih.gov/pubmed/19561590”); 2) Polyphen2_HDIV and Polyphen2_HVAR (available at the world wide web at “ncbi.nlm.nih.gov/pubmed/20354512”); 3) LRT (available at the world wide web at “ncbi.nlm.nih.gov/pubmed/19602639”); and 4) MutationTaster (available at the world wide web at “ncbi.nlm.nih.gov/pubmed/20676075”).

### Statistical Analysis

Genes and Genetic Variants of Interest: This study focused on the association with clinical outcomes of LOF genetic variants in the PCSK1 and MC4R genes.

Frequency of variants in the genes of interest: For each individual of European ancestry with whole-exome sequence data, genotype information from all rare (minor allele frequency (MAF)<1%) LOF or LOF plus missense (M2, M3 or M4) variants in PCSK1 or MC4R was collapsed into a single composite genotype, such that: 1) individuals who were homozygous for the human genome reference allele (R) for all LOF variants in the gene of interest were considered homozygous for the reference allele (“RR” composite genotype), 2) individuals who were heterozygous for at least 1 LOF variant in the gene of interest were considered heterozygous (“RA” composite genotype), and 3) individuals with 2 copies of the alternative allele (A) for the same LOF variant were considered homozygous for the alternative allele (“AA” composite genotype). Variants were not phased, thus any individuals who were heterozygotes for 2 or more LOF variants (i.e., compound heterozygotes) was considered heterozygous (“RA” composite genotype). Given the rarity of compound heterozygous carriers of LOF variants, this is highly unlikely to affect any association results.

The frequency of LOF variants in a gene for a given study (GHS-VCRome, GHS-IDT, and UKB) was then estimated as (N_RA+2*N_AA)/(2*N), where N_RA is the number of individuals with a “RA” composite genotype, N_AA is the number of individuals with a “AA” composite genotype, and N is the total number of individuals with a non-missing composite genotype.

Association Between Genetic Variants and Clinical Outcomes of Interest: Given the rarity of each individual LOF or missense variant, the association analyses was restricted to the composite genotype combining all LOF missense variants in a given gene (as described above), instead of testing the association for individual LOF or missense variants, one at a time.

For quantitative traits, association analyses were performed by fitting the linear mixed model implemented in BOLT-LMM v2.3.4 (Loh et al., Nat. Genet., 2018, 50, 906-908). For the model-fitting step of BOLT-LMM, variants with a MAF>0.5% from the exome sequencing data were used. Age, age², sex, age-by-sex, and 10 ancestry-informative principal components were included as covariates. All quantitative traits were normalized using a rank-based inverse-normal transformation within each sex prior to the BOLT-LMM association analysis. Analyses were performed separately for GHS-VCRome, GHS-IDT, and UKB. Association results were combined across the GHS-VCRome and GHS-IDT sub-studies with METAL using an inverse-variance weighted meta-analysis approach.

Table 2 summarizes the association with body mass index of the burden of predicted loss of function (pLOF) variants or pLOF plus missense variants in PCSK1. The results are from a meta-analysis including 263,183 participants in the UKB or GHS studies.

TABLE 2

| | Alternative | Per allele beta | | Number of |
|---|---|---|---|---|
| | allele | (95% Cl) | | participants by |
| | frequency, | in SDs of body | | genotype |
| Exposure | % | mass index | p-value | (RR\|RA\|AA) |
| pLOF-M1.1 | 0.03% | 0.46 | 1.2 × 10⁻⁰⁸ | 263,040\|143\|0 |
| | | (0.30, 0.62) | | |
| pLOF or predicted- | 0.72% | 0.07 | 4.3 × 10⁻⁰⁶ | 259,405\|3,777\|1 |
| deleterious | | (0.04, 0.10) | | |
| missense variants-M2.1 | | | | |
| pLOF or predicted | 0.24% | 0.10 | 4.5 × 10⁻⁰⁴ | 261,927\|1,256\|0 |
| deleterious | | (0.04, 0.15) | | |
| missense variants-M3.1 | | | | |
| pLOF or predicted- | 0.40% | 0.07 | 5.8 × 10⁻⁰⁴ | 261,078\|2,105\|0 |
| deleterious | | (0.03, 0.11) | | |
| missense variants-M4.1 | | | | |

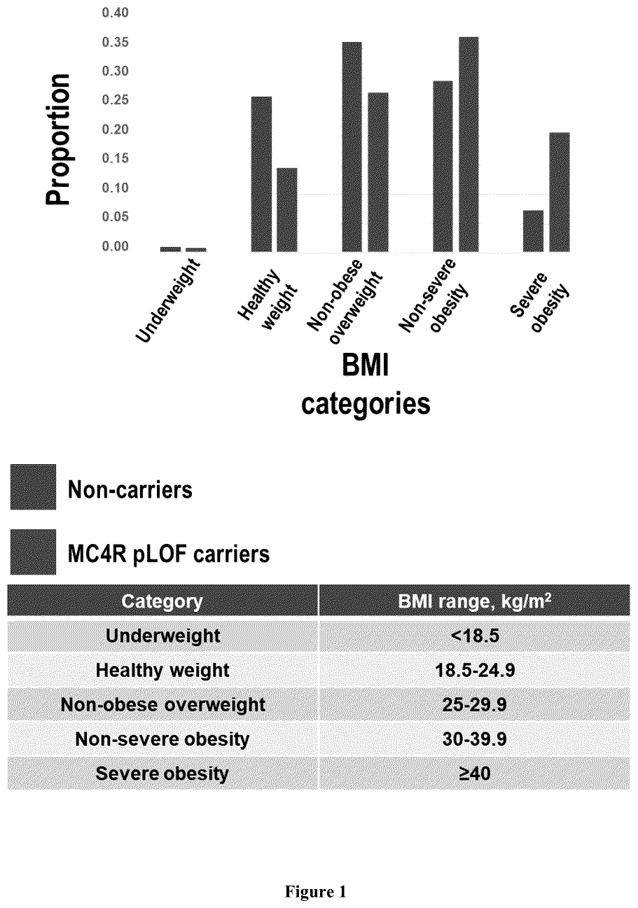

FIG. 1 shows the proportion individuals in different categories of BMI for carriers vs non-carriers of PCSK1 pLOF genetic variants.

Tables 3-6 (M1-M4) list PCSK1 variants included in the association analyses reported in Table 1 (ENST00000311106.8 encompassing chr5:96,390,333-96,433,248 in GRCh38/hg38 human genome assembly; ENSG00000175426.11).

TABLE 3

| VARIANT | AAF | M1 | M2 | M3 | M4 | ANNOTATION | IS_LOF |
|---|---|---|---|---|---|---|---|
| 5:96393172:A:C | 0.0000085 | TRUE | TRUE | TRUE | TRUE | STOP_GAINED | TRUE |
| 5:96393258:G:A | 0.000007 | TRUE | TRUE | TRUE | TRUE | STOP_GAINED | TRUE |
| 5:96393366:G:A | 0.0000194 | TRUE | TRUE | TRUE | TRUE | STOP_GAINED | TRUE |
| 5:96394941:G:A | 0.0000035 | TRUE | TRUE | TRUE | TRUE | STOP_GAINED | TRUE |
| 5:96394988:C:T | 0.0000065 | TRUE | TRUE | TRUE | TRUE | STOP_GAINED | TRUE |
| 5:96397351:CAA:C | 0.0000194 | TRUE | TRUE | TRUE | TRUE | FRAMESHIFT | TRUE |
| 5:96397358:C:T | 0.0000085 | TRUE | TRUE | TRUE | TRUE | STOP_GAINED | TRUE |
| 5:96397377:C:A | 0.0000065 | TRUE | TRUE | TRUE | TRUE | STOP_GAINED | TRUE |
| 5:96397382:C:T | 0.0000035 | TRUE | TRUE | TRUE | TRUE | STOP_GAINED | TRUE |
| 5:96397390:AAC:A | 0.0000065 | TRUE | TRUE | TRUE | TRUE | FRAMESHIFT | TRUE |
| 5:96397440:C:A | 0.0000065 | TRUE | TRUE | TRUE | TRUE | STOP_GAINED | TRUE |
| 5:96398877:A:G | 0.0000065 | TRUE | TRUE | TRUE | TRUE | SPLICE_DONOR | TRUE |
| 5:96398887:GAAGT:G | 0.0000085 | TRUE | TRUE | TRUE | TRUE | FRAMESHIFT | TRUE |
| 5:96398922:A:C | 0.0000035 | TRUE | TRUE | TRUE | TRUE | STOP_GAINED | TRUE |
| 5:96399952:C:A | 0.0000065 | TRUE | TRUE | TRUE | TRUE | SPLICE_DONOR | TRUE |
| 5:96399993:T:A | 0.0000105 | TRUE | TRUE | TRUE | TRUE | STOP_GAINED | TRUE |
| 5:96400071:G:A | 0.000007 | TRUE | TRUE | TRUE | TRUE | STOP_GAINED | TRUE |
| 5:96400106:C:T | 0.000013 | TRUE | TRUE | TRUE | TRUE | STOP_GAINED | TRUE |
| 5:96400170:G:A | 0.0000175 | TRUE | TRUE | TRUE | TRUE | STOP_GAINED | TRUE |
| 5:96408221:A:C | 0.0000035 | TRUE | TRUE | TRUE | TRUE | SPLICE_DONOR | TRUE |
| 5:96408288:C:CA | 0.0000065 | TRUE | TRUE | TRUE | TRUE | FRAMESHIFT | TRUE |
| 5:96408299:A:AG | 0.0000065 | TRUE | TRUE | TRUE | TRUE | FRAMESHIFT | TRUE |
| 5:96408310:A:AG | 0.0000035 | TRUE | TRUE | TRUE | TRUE | FRAMESHIFT | TRUE |
| 5:96408324:C:G | 0.0000035 | TRUE | TRUE | TRUE | TRUE | SPLICE_ACCEPTOR | TRUE |
| 5:96410771:T:TA | 0.0000105 | TRUE | TRUE | TRUE | TRUE | SPLICE_DONOR | TRUE |
| 5:96410773:C:T | 0.0000065 | TRUE | TRUE | TRUE | TRUE | SPLICE_DONOR | TRUE |
| 5:96410797:C:A | 0.000007 | TRUE | TRUE | TRUE | TRUE | STOP_GAINED | TRUE |
| 5:96410840:G:C | 0.000028 | TRUE | TRUE | TRUE | TRUE | STOP_GAINED | TRUE |
| 5:96410840:G:T | 0.0000065 | TRUE | TRUE | TRUE | TRUE | STOP_GAINED | TRUE |
| 5:96410844:CAGGGGGAT:C | 0.000007 | TRUE | TRUE | TRUE | TRUE | FRAMESHIFT | TRUE |
| 5:96410910:T:TC | 0.000013 | TRUE | TRUE | TRUE | TRUE | FRAMESHIFT | TRUE |
| 5:96410935:G:GC | 0.0000035 | TRUE | TRUE | TRUE | TRUE | FRAMESHIFT | TRUE |
| 5:96410974:TC:T | 0.0000712 | TRUE | TRUE | TRUE | TRUE | FRAMESHIFT | TRUE |
| 5:96410983:T:TC | 0.0000035 | TRUE | TRUE | TRUE | TRUE | FRAMESHIFT | TRUE |
| 5:96412437:C:CA | 0.0000065 | TRUE | TRUE | TRUE | TRUE | FRAMESHIFT | TRUE |
| 5:96412452:C:A | 0.0000105 | TRUE | TRUE | TRUE | TRUE | STOP_GAINED | TRUE |
| 5:96412454:A:AT | 0.0000065 | TRUE | TRUE | TRUE | TRUE | FRAMESHIFT | TRUE |
| 5:96416067:G:T | 0.0000035 | TRUE | TRUE | TRUE | TRUE | STOP_GAINED | TRUE |
| 5:96416072:T:A | 0.0000035 | TRUE | TRUE | TRUE | TRUE | STOP_GAINED | TRUE |
| 5:96421879:CT:C | 0.0000035 | TRUE | TRUE | TRUE | TRUE | FRAMESHIFT | TRUE |
| 5:96421881:T:A | 0.0000035 | TRUE | TRUE | TRUE | TRUE | STOP_GAINED | TRUE |
| 5:96421893:TG:T | 0.0000065 | TRUE | TRUE | TRUE | TRUE | FRAMESHIFT | TRUE |
| 5:96421905:G:A | 0.0000194 | TRUE | TRUE | TRUE | TRUE | STOP_GAINED | TRUE |
| 5:96421940:TA:T | 0.0000065 | TRUE | TRUE | TRUE | TRUE | FRAMESHIFT | TRUE |
| 5:96421951:TG:T | 0.0000065 | TRUE | TRUE | TRUE | TRUE | FRAMESHIFT | TRUE |
| 5:96421954:AT:A | 0.0000065 | TRUE | TRUE | TRUE | TRUE | FRAMESHIFT | TRUE |
| 5:96423354:CA:C | 0.0000065 | TRUE | TRUE | TRUE | TRUE | FRAMESHIFT | TRUE |
| 5:96423401:CAA:C | 0.0000035 | TRUE | TRUE | TRUE | TRUE | FRAMESHIFT | TRUE |
| 5:96425819:C:G | 0.0000035 | TRUE | TRUE | TRUE | TRUE | SPLICE_DONOR | TRUE |
| 5:96425827:C:T | 0.0000105 | TRUE | TRUE | TRUE | TRUE | STOP_GAINED | TRUE |
| 5:96425843:TG:T | 0.000013 | TRUE | TRUE | TRUE | TRUE | FRAMESHIFT | TRUE |
| 5:96425891:T:A | 0.0000035 | TRUE | TRUE | TRUE | TRUE | STOP_GAINED | TRUE |
| 5:96425892:AC:A | 0.000007 | TRUE | TRUE | TRUE | TRUE | FRAMESHIFT | TRUE |
| 5:96429256:CT:C | 0.0000065 | TRUE | TRUE | TRUE | TRUE | FRAMESHIFT | TRUE |
| 5:96429260:G:A | 0.0000065 | TRUE | TRUE | TRUE | TRUE | STOP_GAINED | TRUE |
| 5:96429269:TG:T | 0.000013 | TRUE | TRUE | TRUE | TRUE | FRAMESHIFT | TRUE |
| 5:96429310:G:T | 0.000007 | TRUE | TRUE | TRUE | TRUE | STOP_GAINED | TRUE |
| 5:96429319:T:C | 0.000007 | TRUE | TRUE | TRUE | TRUE | SPLICE_ACCEPTOR | TRUE |
| 5:96432082:C:T | 0.0000035 | TRUE | TRUE | TRUE | TRUE | SPLICE_DONOR | TRUE |
| 5:96432861:A:C | 0.0000065 | TRUE | TRUE | TRUE | TRUE | SPLICE_DONOR | TRUE |
| 5:96432869:CA:C | 0.0000085 | TRUE | TRUE | TRUE | TRUE | FRAMESHIFT | TRUE |
| 5:96432913:C:A | 0.0000035 | TRUE | TRUE | TRUE | TRUE | STOP_GAINED | TRUE |
| 5:96432957:CT:C | 0.0000065 | TRUE | TRUE | TRUE | TRUE | FRAMESHIFT | TRUE |
| 5:96432971:AC:A | 0.0000259 | TRUE | TRUE | TRUE | TRUE | FRAMESHIFT | TRUE |
| 5:96432981:G:GCA | 0.0000035 | TRUE | TRUE | TRUE | TRUE | FRAMESHIFT | TRUE |
| 5:96432993:CAA:C | 0.0000065 | TRUE | TRUE | TRUE | TRUE | FRAMESHIFT | TRUE |
| 5:96433040:C:T | 0.0000065 | TRUE | TRUE | TRUE | TRUE | START_LOST | TRUE |
| 5:96433041:A:G | 0.0000035 | TRUE | TRUE | TRUE | TRUE | START_LOST | TRUE |

TABLE 4

| VARIANT | AAF | M1 | M2 | M3 | M4 | ANNOTATION | IS_LOF |
|---|---|---|---|---|---|---|---|
| 5:96393012:C:T | 0.0000175 | FALSE | TRUE | FALSE | TRUE | MISSENSE | FALSE |
| 5:96393013:A:T | 0.0000035 | FALSE | TRUE | FALSE | FALSE | MISSENSE | FALSE |
| 5:96393014:T:C | 0.0000035 | FALSE | TRUE | FALSE | FALSE | MISSENSE | FALSE |
| 5:96393026:A:C | 0.0000035 | FALSE | TRUE | FALSE | TRUE | MISSENSE | FALSE |
| 5:96393027:C:T | 0.0000175 | FALSE | TRUE | FALSE | TRUE | MISSENSE | FALSE |
| 5:96393044:C:T | 0.0000245 | FALSE | TRUE | TRUE | TRUE | MISSENSE | FALSE |
| 5:96393045:G:A | 0.0002452 | FALSE | TRUE | TRUE | TRUE | MISSENSE | FALSE |
| 5:96393048:C:T | 0.0000420 | FALSE | TRUE | TRUE | TRUE | MISSENSE | FALSE |
| 5:96393050:T:C | 0.0000070 | FALSE | TRUE | TRUE | TRUE | MISSENSE | FALSE |
| 5:96393053:C:T | 0.0000035 | FALSE | TRUE | TRUE | TRUE | MISSENSE | FALSE |
| 5:96393057:G:T | 0.0000065 | FALSE | TRUE | TRUE | TRUE | MISSENSE | FALSE |
| 5:96393065:G:A | 0.0000194 | FALSE | TRUE | TRUE | TRUE | MISSENSE | FALSE |
| 5:96393077:T:C | 0.0000035 | FALSE | TRUE | TRUE | TRUE | MISSENSE | FALSE |
| 5:96393078:A:G | 0.0000035 | FALSE | TRUE | TRUE | TRUE | MISSENSE | FALSE |
| 5:96393083:A:C | 0.0000035 | FALSE | TRUE | FALSE | TRUE | MISSENSE | FALSE |
| 5:96393084:C:T | 0.0000065 | FALSE | TRUE | FALSE | FALSE | MISSENSE | FALSE |
| 5:96393101:T:C | 0.0000070 | FALSE | TRUE | TRUE | TRUE | MISSENSE | FALSE |
| 5:96393104:A:C | 0.0000035 | FALSE | TRUE | TRUE | TRUE | MISSENSE | FALSE |
| 5:96393105:G:C | 0.0000035 | FALSE | TRUE | FALSE | TRUE | MISSENSE | FALSE |
| 5:96393105:G:T | 0.0000035 | FALSE | TRUE | TRUE | TRUE | MISSENSE | FALSE |
| 5:96393119:T:C | 0.0000280 | FALSE | TRUE | FALSE | FALSE | MISSENSE | FALSE |
| 5:96393134:G:C | 0.0000085 | FALSE | TRUE | FALSE | TRUE | MISSENSE | FALSE |
| 5:96393155:G:A | 0.0000035 | FALSE | TRUE | TRUE | TRUE | MISSENSE | FALSE |
| 5:96393156:C:T | 0.0000035 | FALSE | TRUE | TRUE | TRUE | MISSENSE | FALSE |
| 5:96393159:C:T | 0.0000140 | FALSE | TRUE | FALSE | TRUE | MISSENSE | FALSE |
| 5:96393168:T:C | 0.0000070 | FALSE | TRUE | FALSE | FALSE | MISSENSE | FALSE |
| 5:96393169:T:G | 0.0000035 | FALSE | TRUE | FALSE | TRUE | MISSENSE | FALSE |
| 5:96393171:C:G | 0.0000035 | FALSE | TRUE | FALSE | FALSE | MISSENSE | FALSE |
| 5:96393172:A:C | 0.0000085 | TRUE | TRUE | TRUE | TRUE | STOP_GAINED | TRUE |
| 5:96393179:A:C | 0.0000105 | FALSE | TRUE | FALSE | TRUE | MISSENSE | FALSE |
| 5:96393181:G:T | 0.0000035 | FALSE | TRUE | FALSE | FALSE | MISSENSE | FALSE |
| 5:96393183:T:C | 0.0000085 | FALSE | TRUE | FALSE | FALSE | MISSENSE | FALSE |
| 5:96393183:T:G | 0.0000065 | FALSE | TRUE | FALSE | FALSE | MISSENSE | FALSE |
| 5:96393186:G:A | 0.0000085 | FALSE | TRUE | FALSE | TRUE | MISSENSE | FALSE |
| 5:96393187:C:G | 0.0000035 | FALSE | TRUE | FALSE | TRUE | MISSENSE | FALSE |
| 5:96393192:C:A | 0.0000085 | FALSE | TRUE | FALSE | FALSE | MISSENSE | FALSE |
| 5:96393198:G:A | 0.0000130 | FALSE | TRUE | FALSE | FALSE | MISSENSE | FALSE |
| 5:96393210:G:A | 0.0000035 | FALSE | TRUE | FALSE | TRUE | MISSENSE | FALSE |
| 5:96393212:G:A | 0.0000175 | FALSE | TRUE | FALSE | FALSE | MISSENSE | FALSE |
| 5:96393224:G:A | 0.0000070 | FALSE | TRUE | FALSE | FALSE | MISSENSE | FALSE |
| 5:96393225:G:T | 0.0000140 | FALSE | TRUE | FALSE | FALSE | MISSENSE | FALSE |
| 5:96393236:C:T | 0.0000194 | FALSE | TRUE | FALSE | FALSE | MISSENSE | FALSE |
| 5:96393239:A:G | 0.0000140 | FALSE | TRUE | TRUE | TRUE | MISSENSE | FALSE |
| 5:96393257:C:A | 0.0000065 | FALSE | TRUE | FALSE | TRUE | MISSENSE | FALSE |
| 5:96393257:C:T | 0.0000140 | FALSE | TRUE | FALSE | TRUE | MISSENSE | FALSE |
| 5:96393258:G:A | 0.0000070 | TRUE | TRUE | TRUE | TRUE | STOP_GAINED | TRUE |
| 5:96393269:T:C | 0.0000035 | FALSE | TRUE | FALSE | FALSE | MISSENSE | FALSE |
| 5:96393272:G:A | 0.0000035 | FALSE | TRUE | FALSE | TRUE | MISSENSE | FALSE |
| 5:96393281:C:G | 0.0000065 | FALSE | TRUE | FALSE | FALSE | MISSENSE | FALSE |
| 5:96393281:C:T | 0.0000035 | FALSE | TRUE | FALSE | FALSE | MISSENSE | FALSE |
| 5:96393284:T:C | 0.0000065 | FALSE | TRUE | FALSE | TRUE | MISSENSE | FALSE |
| 5:96393285:C:T | 0.0000130 | FALSE | TRUE | FALSE | TRUE | MISSENSE | FALSE |
| 5:96393300:T:A | 0.0000065 | FALSE | TRUE | FALSE | TRUE | MISSENSE | FALSE |
| 5:96393302:C:T | 0.0000771 | FALSE | TRUE | FALSE | FALSE | MISSENSE | FALSE |
| 5:96393303:G:A | 0.0000140 | FALSE | TRUE | FALSE | TRUE | MISSENSE | FALSE |
| 5:96393305:C:A | 0.0000210 | FALSE | TRUE | FALSE | FALSE | MISSENSE | FALSE |
| 5:96393308:C:A | 0.0000453 | FALSE | TRUE | FALSE | TRUE | MISSENSE | FALSE |
| 5:96393312:C:T | 0.0000350 | FALSE | TRUE | FALSE | FALSE | MISSENSE | FALSE |
| 5:96393324:T:G | 0.0000035 | FALSE | TRUE | FALSE | FALSE | MISSENSE | FALSE |
| 5:96393328:G:T | 0.0000389 | FALSE | TRUE | FALSE | FALSE | MISSENSE | FALSE |
| 5:96393342:G:T | 0.0000085 | FALSE | TRUE | FALSE | FALSE | MISSENSE | FALSE |
| 5:96393344:G:T | 0.0000065 | FALSE | TRUE | FALSE | FALSE | MISSENSE | FALSE |
| 5:96393345:T:C | 0.0030547 | FALSE | TRUE | FALSE | FALSE | MISSENSE | FALSE |
| 5:96393347:T:C | 0.0000065 | FALSE | TRUE | FALSE | FALSE | MISSENSE | FALSE |
| 5:96393363:C:T | 0.0000065 | FALSE | TRUE | FALSE | FALSE | MISSENSE | FALSE |
| 5:96393365:T:C | 0.0000035 | FALSE | TRUE | FALSE | FALSE | MISSENSE | FALSE |
| 5:96393366:G:A | 0.0000194 | TRUE | TRUE | TRUE | TRUE | STOP_GAINED | TRUE |
| 5:96393378:C:G | 0.0000035 | FALSE | TRUE | FALSE | TRUE | MISSENSE | FALSE |
| 5:96394878:C:A | 0.0000070 | FALSE | TRUE | FALSE | TRUE | MISSENSE | FALSE |
| 5:96394882:C:G | 0.0000035 | FALSE | TRUE | FALSE | TRUE | MISSENSE | FALSE |
| 5:96394890:C:A | 0.0000035 | FALSE | TRUE | FALSE | TRUE | MISSENSE | FALSE |
| 5:96394893:C:T | 0.0000035 | FALSE | TRUE | FALSE | TRUE | MISSENSE | FALSE |
| 5:96394901:T:G | 0.0000035 | FALSE | TRUE | FALSE | TRUE | MISSENSE | FALSE |
| 5:96394911:C:T | 0.0000175 | FALSE | TRUE | FALSE | TRUE | MISSENSE | FALSE |
| 5:96394925:G:A | 0.0000175 | FALSE | TRUE | TRUE | TRUE | MISSENSE | FALSE |
| 5:96394934:C:T | 0.0000175 | FALSE | TRUE | TRUE | TRUE | MISSENSE | FALSE |
| 5:96394935:G:A | 0.0000035 | FALSE | TRUE | TRUE | TRUE | MISSENSE | FALSE |
| 5:96394941:G:A | 0.0000035 | TRUE | TRUE | TRUE | TRUE | STOP_GAINED | TRUE |
| 5:96394947:T:C | 0.0000065 | FALSE | TRUE | FALSE | TRUE | MISSENSE | FALSE |
| 5:96394964:G:T | 0.0000065 | FALSE | TRUE | FALSE | TRUE | MISSENSE | FALSE |
| 5:96394973:T:C | 0.0000035 | FALSE | TRUE | TRUE | TRUE | MISSENSE | FALSE |
| 5:96394988:C:T | 0.0000065 | TRUE | TRUE | TRUE | TRUE | STOP_GAINED | TRUE |
| 5:96394997:A:G | 0.0000035 | FALSE | TRUE | TRUE | TRUE | MISSENSE | FALSE |
| 5:96395003:C:T | 0.0000065 | FALSE | TRUE | TRUE | TRUE | MISSENSE | FALSE |
| 5:96395024:G:C | 0.0000065 | FALSE | TRUE | TRUE | TRUE | MISSENSE | FALSE |
| 5:96397344:T:C | 0.0000085 | FALSE | TRUE | FALSE | TRUE | MISSENSE | FALSE |
| 5:96397346:A:T | 0.0000105 | FALSE | TRUE | TRUE | TRUE | MISSENSE | FALSE |
| 5:96397347:T:A | 0.0000455 | FALSE | TRUE | FALSE | TRUE | MISSENSE | FALSE |
| 5:96397351:CAA:C | 0.0000194 | TRUE | TRUE | TRUE | TRUE | FRAMESHIFT | TRUE |
| 5:96397351:C:G | 0.0000254 | FALSE | TRUE | TRUE | TRUE | MISSENSE | FALSE |
| 5:96397357:C:G | 0.0000065 | FALSE | TRUE | TRUE | TRUE | MISSENSE | FALSE |
| 5:96397358:C:T | 0.0000085 | TRUE | TRUE | TRUE | TRUE | STOP_GAINED | TRUE |
| 5:96397362:T:C | 0.0000035 | FALSE | TRUE | FALSE | TRUE | MISSENSE | FALSE |
| 5:96397364:C:A | 0.0000065 | FALSE | TRUE | TRUE | TRUE | MISSENSE | FALSE |
| 5:96397366:T:C | 0.0000130 | FALSE | TRUE | FALSE | FALSE | MISSENSE | FALSE |
| 5:96397367:A:G | 0.0000280 | FALSE | TRUE | FALSE | FALSE | MISSENSE | FALSE |
| 5:96397372:G:T | 0.0000035 | FALSE | TRUE | FALSE | TRUE | MISSENSE | FALSE |
| 5:96397377:C:A | 0.0000065 | TRUE | TRUE | TRUE | TRUE | STOP_GAINED | TRUE |
| 5:96397380:C:T | 0.0000065 | FALSE | TRUE | TRUE | TRUE | MISSENSE | FALSE |
| 5:96397382:C:T | 0.0000035 | TRUE | TRUE | TRUE | TRUE | STOP_GAINED | TRUE |
| 5:96397390:AAC:A | 0.0000065 | TRUE | TRUE | TRUE | TRUE | FRAMESHIFT | TRUE |
| 5:96397392:C:T | 0.0000701 | FALSE | TRUE | FALSE | TRUE | MISSENSE | FALSE |
| 5:96397397:A:G | 0.0000065 | FALSE | TRUE | TRUE | TRUE | MISSENSE | FALSE |
| 5:96397424:G:C | 0.0000210 | FALSE | TRUE | FALSE | TRUE | MISSENSE | FALSE |
| 5:96397431:T:A | 0.0000035 | FALSE | TRUE | FALSE | TRUE | MISSENSE | FALSE |
| 5:96397432:A:T | 0.0000035 | FALSE | TRUE | TRUE | TRUE | MISSENSE | FALSE |
| 5:96397433:T:C | 0.0000070 | FALSE | TRUE | TRUE | TRUE | MISSENSE | FALSE |
| 5:96397436:C:G | 0.0000194 | FALSE | TRUE | FALSE | TRUE | MISSENSE | FALSE |
| 5:96397437:G:A | 0.0000315 | FALSE | TRUE | FALSE | TRUE | MISSENSE | FALSE |
| 5:96397439:T:C | 0.0000035 | FALSE | TRUE | FALSE | TRUE | MISSENSE | FALSE |
| 5:96397440:C:A | 0.0000065 | TRUE | TRUE | TRUE | TRUE | STOP_GAINED | TRUE |
| 5:96397451:A:G | 0.0000035 | FALSE | TRUE | TRUE | TRUE | MISSENSE | FALSE |
| 5:96397461:T:C | 0.0000035 | FALSE | TRUE | FALSE | TRUE | MISSENSE | FALSE |
| 5:96397463:C:T | 0.0000035 | FALSE | TRUE | FALSE | TRUE | MISSENSE | FALSE |
| 5:96398877:A:G | 0.0000065 | TRUE | TRUE | TRUE | TRUE | SPLICE_DONOR | TRUE |
| 5:96398887:GAAGT:G | 0.0000085 | TRUE | TRUE | TRUE | TRUE | FRAMESHIFT | TRUE |
| 5:96398897:T:C | 0.0000035 | FALSE | TRUE | FALSE | TRUE | MISSENSE | FALSE |
| 5:96398902:T:C | 0.0000035 | FALSE | TRUE | FALSE | TRUE | MISSENSE | FALSE |
| 5:96398911:C:A | 0.0000130 | FALSE | TRUE | TRUE | TRUE | MISSENSE | FALSE |
| 5:96398917:C:T | 0.0002172 | FALSE | TRUE | TRUE | TRUE | MISSENSE | FALSE |
| 5:96398920:G:A | 0.0000035 | FALSE | TRUE | FALSE | TRUE | MISSENSE | FALSE |
| 5:96398922:A:C | 0.0000035 | TRUE | TRUE | TRUE | TRUE | STOP_GAINED | TRUE |
| 5:96398929:A:G | 0.0000035 | FALSE | TRUE | TRUE | TRUE | MISSENSE | FALSE |
| 5:96398945:G:T | 0.0000085 | FALSE | TRUE | FALSE | TRUE | MISSENSE | FALSE |
| 5:96398966:T:G | 0.0000070 | FALSE | TRUE | FALSE | TRUE | MISSENSE | FALSE |
| 5:96398969:C:A | 0.0000245 | FALSE | TRUE | FALSE | FALSE | MISSENSE | FALSE |
| 5:96398980:C:T | 0.0000035 | FALSE | TRUE | FALSE | TRUE | MISSENSE | FALSE |
| 5:96398983:T:A | 0.0000085 | FALSE | TRUE | FALSE | TRUE | MISSENSE | FALSE |
| 5:96398992:C:T | 0.0000065 | FALSE | TRUE | FALSE | FALSE | MISSENSE | FALSE |
| 5:96399007:A:G | 0.0000035 | FALSE | TRUE | FALSE | TRUE | MISSENSE | FALSE |
| 5:96399008:T:C | 0.0000065 | FALSE | TRUE | FALSE | TRUE | MISSENSE | FALSE |
| 5:96399952:C:A | 0.0000065 | TRUE | TRUE | TRUE | TRUE | SPLICE_DONOR | TRUE |
| 5:96399958:C:G | 0.0000070 | FALSE | TRUE | FALSE | TRUE | MISSENSE | FALSE |
| 5:96399969:T:C | 0.0000035 | FALSE | TRUE | FALSE | TRUE | MISSENSE | FALSE |
| 5:96399978:C:T | 0.0000245 | FALSE | TRUE | FALSE | TRUE | MISSENSE | FALSE |
| 5:96399980:A:G | 0.0000453 | FALSE | TRUE | FALSE | TRUE | MISSENSE | FALSE |
| 5:96399993:T:A | 0.0000105 | TRUE | TRUE | TRUE | TRUE | STOP_GAINED | TRUE |
| 5:96399994:C:G | 0.0000070 | FALSE | TRUE | FALSE | TRUE | MISSENSE | FALSE |
| 5:96399996:C:T | 0.0000070 | FALSE | TRUE | FALSE | TRUE | MISSENSE | FALSE |
| 5:96399999:G:T | 0.0000175 | FALSE | TRUE | TRUE | TRUE | MISSENSE | FALSE |
| 5:96400002:C:G | 0.0000070 | FALSE | TRUE | FALSE | TRUE | MISSENSE | FALSE |
| 5:96400002:C:T | 0.0000596 | FALSE | TRUE | TRUE | TRUE | MISSENSE | FALSE |
| 5:96400003:G:T | 0.0000070 | FALSE | TRUE | FALSE | FALSE | MISSENSE | FALSE |
| 5:96400004:C:T | 0.0000105 | FALSE | TRUE | FALSE | FALSE | MISSENSE | FALSE |
| 5:96400010:C:G | 0.0000085 | FALSE | TRUE | TRUE | TRUE | MISSENSE | FALSE |
| 5:96400028:A:G | 0.0000065 | FALSE | TRUE | TRUE | TRUE | MISSENSE | FALSE |
| 5:96400032:C:T | 0.0000035 | FALSE | TRUE | FALSE | TRUE | MISSENSE | FALSE |
| 5:96400038:G:C | 0.0000070 | FALSE | TRUE | TRUE | TRUE | MISSENSE | FALSE |
| 5:96400047:C:A | 0.0000035 | FALSE | TRUE | TRUE | TRUE | MISSENSE | FALSE |
| 5:96400049:T:A | 0.0000035 | FALSE | TRUE | TRUE | TRUE | MISSENSE | FALSE |
| 5:96400052:A:G | 0.0000065 | FALSE | TRUE | TRUE | TRUE | MISSENSE | FALSE |
| 5:96400061:A:T | 0.0000035 | FALSE | TRUE | FALSE | TRUE | MISSENSE | FALSE |
| 5:96400070:C:T | 0.0000065 | FALSE | TRUE | FALSE | TRUE | MISSENSE | FALSE |
| 5:96400071:G:A | 0.0000070 | TRUE | TRUE | TRUE | TRUE | STOP_GAINED | TRUE |
| 5:96400091:G:T | 0.0000035 | FALSE | TRUE | TRUE | TRUE | MISSENSE | FALSE |
| 5:96400096:A:T | 0.0000259 | FALSE | TRUE | TRUE | TRUE | MISSENSE | FALSE |
| 5:96400100:T:C | 0.0000085 | FALSE | TRUE | FALSE | TRUE | MISSENSE | FALSE |
| 5:96400106:C:T | 0.0000130 | TRUE | TRUE | TRUE | TRUE | STOP_GAINED | TRUE |
| 5:96400118:T:C | 0.0000085 | FALSE | TRUE | FALSE | FALSE | MISSENSE | FALSE |
| 5:96400128:G:A | 0.0000085 | FALSE | TRUE | FALSE | TRUE | MISSENSE | FALSE |
| 5:96400133:T:G | 0.0000175 | FALSE | TRUE | FALSE | TRUE | MISSENSE | FALSE |
| 5:96400142:G:C | 0.0000035 | FALSE | TRUE | FALSE | TRUE | MISSENSE | FALSE |
| 5:96400146:A:G | 0.0000070 | FALSE | TRUE | FALSE | TRUE | MISSENSE | FALSE |
| 5:96400156:G:C | 0.0000065 | FALSE | TRUE | TRUE | TRUE | MISSENSE | FALSE |
| 5:96400169:C:T | 0.0000035 | FALSE | TRUE | TRUE | TRUE | MISSENSE | FALSE |
| 5:96400170:G:A | 0.0000175 | TRUE | TRUE | TRUE | TRUE | STOP_GAINED | TRUE |
| 5:96408221:A:C | 0.0000035 | TRUE | TRUE | TRUE | TRUE | SPLICE_DONOR | TRUE |
| 5:96408226:G:T | 0.0000280 | FALSE | TRUE | TRUE | TRUE | MISSENSE | FALSE |
| 5:96408232:A:G | 0.0000130 | FALSE | TRUE | TRUE | TRUE | MISSENSE | FALSE |
| 5:96408238:A:G | 0.0000035 | FALSE | TRUE | TRUE | TRUE | MISSENSE | FALSE |
| 5:96408242:C:G | 0.0000065 | FALSE | TRUE | TRUE | TRUE | MISSENSE | FALSE |
| 5:96408242:C:T | 0.0000070 | FALSE | TRUE | TRUE | TRUE | MISSENSE | FALSE |
| 5:96408244:A:G | 0.0000280 | FALSE | TRUE | TRUE | TRUE | MISSENSE | FALSE |
| 5:96408251:C:T | 0.0000035 | FALSE | TRUE | TRUE | TRUE | MISSENSE | FALSE |
| 5:96408254:C:T | 0.0000035 | FALSE | TRUE | TRUE | TRUE | MISSENSE | FALSE |
| 5:96408256:G:T | 0.0000065 | FALSE | TRUE | TRUE | TRUE | MISSENSE | FALSE |
| 5:96408271:G:A | 0.0000035 | FALSE | TRUE | FALSE | TRUE | MISSENSE | FALSE |
| 5:96408272:C:T | 0.0000065 | FALSE | TRUE | TRUE | TRUE | MISSENSE | FALSE |
| 5:96408274:G:A | 0.0000105 | FALSE | TRUE | TRUE | TRUE | MISSENSE | FALSE |
| 5:96408283:G:C | 0.0000194 | FALSE | TRUE | TRUE | TRUE | MISSENSE | FALSE |
| 5:96408285:G:T | 0.0000085 | FALSE | TRUE | TRUE | TRUE | MISSENSE | FALSE |
| 5:96408288:C:CA | 0.0000065 | TRUE | TRUE | TRUE | TRUE | FRAMESHIFT | TRUE |
| 5:96408289:G:A | 0.0000666 | FALSE | TRUE | FALSE | TRUE | MISSENSE | FALSE |
| 5:96408292:T:A | 0.0000065 | FALSE | TRUE | FALSE | TRUE | MISSENSE | FALSE |
| 5:96408293:C:T | 0.0000065 | FALSE | TRUE | FALSE | FALSE | MISSENSE | FALSE |
| 5:96408295:G:A | 0.0000035 | FALSE | TRUE | TRUE | TRUE | MISSENSE | FALSE |
| 5:96408296:T:A | 0.0000065 | FALSE | TRUE | TRUE | TRUE | MISSENSE | FALSE |
| 5:96408299:A:AG | 0.0000065 | TRUE | TRUE | TRUE | TRUE | FRAMESHIFT | TRUE |
| 5:96408303:A:T | 0.0000085 | FALSE | TRUE | FALSE | TRUE | MISSENSE | FALSE |
| 5:96408310:A:AG | 0.0000035 | TRUE | TRUE | TRUE | TRUE | FRAMESHIFT | TRUE |
| 5:96408312:G:C | 0.0000085 | FALSE | TRUE | TRUE | TRUE | MISSENSE | FALSE |
| 5:96408313:T:C | 0.0000065 | FALSE | TRUE | TRUE | TRUE | MISSENSE | FALSE |
| 5:96408316:G:A | 0.0000175 | FALSE | TRUE | FALSE | TRUE | MISSENSE | FALSE |
| 5:96408322:G:A | 0.0000070 | FALSE | TRUE | FALSE | TRUE | MISSENSE | FALSE |
| 5:96408323:T:C | 0.0001401 | FALSE | TRUE | FALSE | TRUE | MISSENSE | FALSE |
| 5:96408324:C:G | 0.0000035 | TRUE | TRUE | TRUE | TRUE | SPLICE_ACCEPTOR | TRUE |
| 5:96410771:T:TA | 0.0000105 | TRUE | TRUE | TRUE | TRUE | SPLICE_DONOR | TRUE |
| 5:96410773:C:T | 0.0000065 | TRUE | TRUE | TRUE | TRUE | SPLICE_DONOR | TRUE |
| 5:96410776:T:C | 0.0000035 | FALSE | TRUE | FALSE | TRUE | MISSENSE | FALSE |
| 5:96410785:C:T | 0.0000035 | FALSE | TRUE | TRUE | TRUE | MISSENSE | FALSE |
| 5:96410794:C:G | 0.0000070 | FALSE | TRUE | FALSE | TRUE | MISSENSE | FALSE |
| 5:96410797:C:A | 0.0000070 | TRUE | TRUE | TRUE | TRUE | STOP_GAINED | TRUE |
| 5:96410797:C:T | 0.0000035 | FALSE | TRUE | TRUE | TRUE | MISSENSE | FALSE |
| 5:96410808:G:C | 0.0000035 | FALSE | TRUE | TRUE | TRUE | MISSENSE | FALSE |
| 5:96410814:G:A | 0.0000035 | FALSE | TRUE | TRUE | TRUE | MISSENSE | FALSE |
| 5:96410818:G:T | 0.0000065 | FALSE | TRUE | TRUE | TRUE | MISSENSE | FALSE |
| 5:96410826:G:A | 0.0000035 | FALSE | TRUE | TRUE | TRUE | MISSENSE | FALSE |
| 5:96410839:C:A | 0.0000085 | FALSE | TRUE | FALSE | TRUE | MISSENSE | FALSE |
| 5:96410839:C:T | 0.0000105 | FALSE | TRUE | FALSE | TRUE | MISSENSE | FALSE |
| 5:96410840:G:C | 0.0000280 | TRUE | TRUE | TRUE | TRUE | STOP_GAINED | TRUE |
| 5:96410840:G:T | 0.0000065 | TRUE | TRUE | TRUE | TRUE | STOP_GAINED | TRUE |
| 5:96410842:A:G | 0.0000085 | FALSE | TRUE | TRUE | TRUE | MISSENSE | FALSE |
| 5:96410844:CAGGGGGAT:C | 0.0000070 | TRUE | TRUE | TRUE | TRUE | FRAMESHIFT | TRUE |
| 5:96410845:A:C | 0.0000210 | FALSE | TRUE | TRUE | TRUE | MISSENSE | FALSE |
| 5:96410856:C:A | 0.0000140 | FALSE | TRUE | TRUE | TRUE | MISSENSE | FALSE |
| 5:96410856:C:T | 0.0000070 | FALSE | TRUE | TRUE | TRUE | MISSENSE | FALSE |
| 5:96410857:C:A | 0.0000065 | FALSE | TRUE | TRUE | TRUE | MISSENSE | FALSE |
| 5:96410875:T:C | 0.0000035 | FALSE | TRUE | FALSE | TRUE | MISSENSE | FALSE |
| 5:96410878:T:A | 0.0000070 | FALSE | TRUE | TRUE | TRUE | MISSENSE | FALSE |
| 5:96410883:A:G | 0.0000105 | FALSE | TRUE | TRUE | TRUE | MISSENSE | FALSE |
| 5:96410887:T:C | 0.0000065 | FALSE | TRUE | TRUE | TRUE | MISSENSE | FALSE |
| 5:96410895:C:A | 0.0000065 | FALSE | TRUE | TRUE | TRUE | MISSENSE | FALSE |
| 5:96410901:G:A | 0.0000035 | FALSE | TRUE | TRUE | TRUE | MISSENSE | FALSE |
| 5:96410907:C:A | 0.0000035 | FALSE | TRUE | TRUE | TRUE | MISSENSE | FALSE |
| 5:96410907:C:T | 0.0000035 | FALSE | TRUE | TRUE | TRUE | MISSENSE | FALSE |
| 5:96410910:T:TC | 0.0000130 | TRUE | TRUE | TRUE | TRUE | FRAMESHIFT | TRUE |
| 5:96410913:C:T | 0.0000065 | FALSE | TRUE | TRUE | TRUE | MISSENSE | FALSE |
| 5:96410929:C:T | 0.0000453 | FALSE | TRUE | FALSE | TRUE | MISSENSE | FALSE |
| 5:96410934:C:T | 0.0000035 | FALSE | TRUE | FALSE | TRUE | MISSENSE | FALSE |
| 5:96410935:G:A | 0.0000035 | FALSE | TRUE | TRUE | TRUE | MISSENSE | FALSE |
| 5:96410935:G:GC | 0.0000035 | TRUE | TRUE | TRUE | TRUE | FRAMESHIFT | TRUE |
| 5:96410940:C:A | 0.0000035 | FALSE | TRUE | TRUE | TRUE | MISSENSE | FALSE |
| 5:96410941:C:T | 0.0000085 | FALSE | TRUE | TRUE | TRUE | MISSENSE | FALSE |
| 5:96410946:C:T | 0.0000105 | FALSE | TRUE | TRUE | TRUE | MISSENSE | FALSE |
| 5:96410949:G:A | 0.0000175 | FALSE | TRUE | FALSE | TRUE | MISSENSE | FALSE |
| 5:96410959:C:T | 0.0000525 | FALSE | TRUE | FALSE | TRUE | MISSENSE | FALSE |
| 5:96410965:T:G | 0.0000070 | FALSE | TRUE | FALSE | TRUE | MISSENSE | FALSE |
| 5:96410970:C:T | 0.0000035 | FALSE | TRUE | TRUE | TRUE | MISSENSE | FALSE |
| 5:96410974:TC:T | 0.0000712 | TRUE | TRUE | TRUE | TRUE | FRAMESHIFT | TRUE |
| 5:96410976:C:G | 0.0000065 | FALSE | TRUE | TRUE | TRUE | MISSENSE | FALSE |
| 5:96410983:T:TC | 0.0000035 | TRUE | TRUE | TRUE | TRUE | FRAMESHIFT | TRUE |
| 5:96410985:C:T | 0.0000065 | FALSE | TRUE | TRUE | TRUE | MISSENSE | FALSE |
| 5:96412322:T:C | 0.0000130 | FALSE | TRUE | FALSE | TRUE | MISSENSE | FALSE |
| 5:96412328:C:T | 0.0000035 | FALSE | TRUE | TRUE | TRUE | MISSENSE | FALSE |
| 5:96412332:A:G | 0.0000130 | FALSE | TRUE | FALSE | TRUE | MISSENSE | FALSE |
| 5:96412355:C:T | 0.0000065 | FALSE | TRUE | FALSE | TRUE | MISSENSE | FALSE |
| 5:96412356:G:A | 0.0000175 | FALSE | TRUE | TRUE | TRUE | MISSENSE | FALSE |
| 5:96412362:G:A | 0.0000130 | FALSE | TRUE | TRUE | TRUE | MISSENSE | FALSE |
| 5:96412362:G:T | 0.0000035 | FALSE | TRUE | TRUE | TRUE | MISSENSE | FALSE |
| 5:96412364:C:G | 0.0000169 | FALSE | TRUE | TRUE | TRUE | MISSENSE | FALSE |
| 5:96412379:C:G | 0.0000070 | FALSE | TRUE | TRUE | TRUE | MISSENSE | FALSE |
| 5:96412382:T:C | 0.0000065 | FALSE | TRUE | TRUE | TRUE | MISSENSE | FALSE |
| 5:96412385:T:C | 0.0000169 | FALSE | TRUE | TRUE | TRUE | MISSENSE | FALSE |
| 5:96412395:C:T | 0.0000065 | FALSE | TRUE | TRUE | TRUE | MISSENSE | FALSE |
| 5:96412400:C:T | 0.0000085 | FALSE | TRUE | TRUE | TRUE | MISSENSE | FALSE |
| 5:96412416:C:T | 0.0000130 | FALSE | TRUE | TRUE | TRUE | MISSENSE | FALSE |
| 5:96412418:A:G | 0.0000140 | FALSE | TRUE | TRUE | TRUE | MISSENSE | FALSE |
| 5:96412419:C:T | 0.0000070 | FALSE | TRUE | TRUE | TRUE | MISSENSE | FALSE |
| 5:96412420:G:T | 0.0000105 | FALSE | TRUE | TRUE | TRUE | MISSENSE | FALSE |
| 5:96412425:C:T | 0.0000085 | FALSE | TRUE | FALSE | TRUE | MISSENSE | FALSE |
| 5:96412436:C:G | 0.0000065 | FALSE | TRUE | TRUE | TRUE | MISSENSE | FALSE |
| 5:96412436:C:T | 0.0000070 | FALSE | TRUE | TRUE | TRUE | MISSENSE | FALSE |
| 5:96412437:C:CA | 0.0000065 | TRUE | TRUE | TRUE | TRUE | FRAMESHIFT | TRUE |
| 5:96412437:C:T | 0.0000035 | FALSE | TRUE | TRUE | TRUE | MISSENSE | FALSE |
| 5:96412439:A:G | 0.0000085 | FALSE | TRUE | TRUE | TRUE | MISSENSE | FALSE |
| 5:96412440:T:C | 0.0000065 | FALSE | TRUE | FALSE | TRUE | MISSENSE | FALSE |
| 5:96412443:A:T | 0.0000065 | FALSE | TRUE | TRUE | TRUE | MISSENSE | FALSE |
| 5:96412445:C:T | 0.0000035 | FALSE | TRUE | FALSE | TRUE | MISSENSE | FALSE |
| 5:96412448:G:A | 0.0000070 | FALSE | TRUE | TRUE | TRUE | MISSENSE | FALSE |
| 5:96412452:C:A | 0.0000105 | TRUE | TRUE | TRUE | TRUE | STOP_GAINED | TRUE |
| 5:96412454:A:AT | 0.0000065 | TRUE | TRUE | TRUE | TRUE | FRAMESHIFT | TRUE |
| 5:96412454:A:G | 0.0000035 | FALSE | TRUE | TRUE | TRUE | MISSENSE | FALSE |
| 5:96412455:T:C | 0.0000712 | FALSE | TRUE | FALSE | TRUE | MISSENSE | FALSE |
| 5:96412463:G:A | 0.0000140 | FALSE | TRUE | TRUE | TRUE | MISSENSE | FALSE |
| 5:96412464:T:A | 0.0000035 | FALSE | TRUE | TRUE | TRUE | MISSENSE | FALSE |
| 5:96412480:C:G | 0.0000035 | FALSE | TRUE | TRUE | TRUE | MISSENSE | FALSE |
| 5:96412484:C:A | 0.0000085 | FALSE | TRUE | TRUE | TRUE | MISSENSE | FALSE |
| 5:96412488:T:C | 0.0000035 | FALSE | TRUE | FALSE | TRUE | MISSENSE | FALSE |
| 5:96416041:T:A | 0.0001296 | FALSE | TRUE | TRUE | TRUE | MISSENSE | FALSE |
| 5:96416041:T:G | 0.0000065 | FALSE | TRUE | TRUE | TRUE | MISSENSE | FALSE |
| 5:96416047:T:C | 0.0000070 | FALSE | TRUE | FALSE | TRUE | MISSENSE | FALSE |
| 5:96416050:T:C | 0.0000130 | FALSE | TRUE | TRUE | TRUE | MISSENSE | FALSE |
| 5:96416051:A:G | 0.0000070 | FALSE | TRUE | TRUE | TRUE | MISSENSE | FALSE |
| 5:96416053:G:A | 0.0000194 | FALSE | TRUE | TRUE | TRUE | MISSENSE | FALSE |
| 5:96416054:C:A | 0.0000035 | FALSE | TRUE | TRUE | TRUE | MISSENSE | FALSE |
| 5:96416054:C:G | 0.0000065 | FALSE | TRUE | TRUE | TRUE | MISSENSE | FALSE |
| 5:96416057:C:T | 0.0000194 | FALSE | TRUE | FALSE | TRUE | MISSENSE | FALSE |
| 5:96416059:C:A | 0.0000105 | FALSE | TRUE | TRUE | TRUE | MISSENSE | FALSE |
| 5:96416062:A:G | 0.0000070 | FALSE | TRUE | FALSE | TRUE | MISSENSE | FALSE |
| 5:96416063:C:T | 0.0000070 | FALSE | TRUE | TRUE | TRUE | MISSENSE | FALSE |
| 5:96416066:C:G | 0.0000035 | FALSE | TRUE | TRUE | TRUE | MISSENSE | FALSE |
| 5:96416066:C:T | 0.0000210 | FALSE | TRUE | TRUE | TRUE | MISSENSE | FALSE |
| 5:96416067:G:T | 0.0000035 | TRUE | TRUE | TRUE | TRUE | STOP_GAINED | TRUE |
| 5:96416068:C:T | 0.0000035 | FALSE | TRUE | TRUE | TRUE | MISSENSE | FALSE |
| 5:96416069:A:G | 0.0000035 | FALSE | TRUE | TRUE | TRUE | MISSENSE | FALSE |
| 5:96416072:T:A | 0.0000035 | TRUE | TRUE | TRUE | TRUE | STOP_GAINED | TRUE |
| 5:96416075:G:A | 0.0000035 | FALSE | TRUE | FALSE | TRUE | MISSENSE | FALSE |
| 5:96416092:G:A | 0.0001787 | FALSE | TRUE | TRUE | TRUE | MISSENSE | FALSE |
| 5:96416105:C:A | 0.0000035 | FALSE | TRUE | TRUE | TRUE | MISSENSE | FALSE |
| 5:96416105:C:T | 0.0000035 | FALSE | TRUE | TRUE | TRUE | MISSENSE | FALSE |
| 5:96416117:C:T | 0.0000035 | FALSE | TRUE | TRUE | TRUE | MISSENSE | FALSE |
| 5:96421879:CT:C | 0.0000035 | TRUE | TRUE | TRUE | TRUE | FRAMESHIFT | TRUE |
| 5:96421881:T:A | 0.0000035 | TRUE | TRUE | TRUE | TRUE | STOP_GAINED | TRUE |
| 5:96421882:G:T | 0.0000105 | FALSE | TRUE | TRUE | TRUE | MISSENSE | FALSE |
| 5:96421887:C:T | 0.0000105 | FALSE | TRUE | FALSE | TRUE | MISSENSE | FALSE |
| 5:96421893:TG:T | 0.0000065 | TRUE | TRUE | TRUE | TRUE | FRAMESHIFT | TRUE |
| 5:96421896:G:A | 0.0000070 | FALSE | TRUE | FALSE | TRUE | MISSENSE | FALSE |
| 5:96421898:T:A | 0.0000070 | FALSE | TRUE | TRUE | TRUE | MISSENSE | FALSE |
| 5:96421902:A:T | 0.0000035 | FALSE | TRUE | TRUE | TRUE | MISSENSE | FALSE |
| 5:96421904:C:T | 0.0000105 | FALSE | TRUE | TRUE | TRUE | MISSENSE | FALSE |
| 5:96421905:G:A | 0.0000194 | TRUE | TRUE | TRUE | TRUE | STOP_GAINED | TRUE |
| 5:96421908:G:A | 0.0000065 | FALSE | TRUE | TRUE | TRUE | MISSENSE | FALSE |
| 5:96421914:G:A | 0.0000194 | FALSE | TRUE | TRUE | TRUE | MISSENSE | FALSE |
| 5:96421916:T:C | 0.0000070 | FALSE | TRUE | TRUE | TRUE | MISSENSE | FALSE |
| 5:96421916:T:G | 0.0000105 | FALSE | TRUE | TRUE | TRUE | MISSENSE | FALSE |
| 5:96421922:T:C | 0.0000035 | FALSE | TRUE | TRUE | TRUE | MISSENSE | FALSE |
| 5:96421932:T:A | 0.0000065 | FALSE | TRUE | TRUE | TRUE | MISSENSE | FALSE |
| 5:96421934:A:C | 0.0000169 | FALSE | TRUE | FALSE | TRUE | MISSENSE | FALSE |
| 5:96421940:TA:T | 0.0000065 | TRUE | TRUE | TRUE | TRUE | FRAMESHIFT | TRUE |
| 5:96421941:A:G | 0.0000350 | FALSE | TRUE | TRUE | TRUE | MISSENSE | FALSE |
| 5:96421946:G:C | 0.0000210 | FALSE | TRUE | TRUE | TRUE | MISSENSE | FALSE |
| 5:96421950:C:G | 0.0000035 | FALSE | TRUE | FALSE | TRUE | MISSENSE | FALSE |
| 5:96421951:TG:T | 0.0000065 | TRUE | TRUE | TRUE | TRUE | FRAMESHIFT | TRUE |
| 5:96421954:AT:A | 0.0000065 | TRUE | TRUE | TRUE | TRUE | FRAMESHIFT | TRUE |
| 5:96423315:A:G | 0.0003818 | FALSE | TRUE | TRUE | TRUE | MISSENSE | FALSE |
| 5:96423317:T:C | 0.0000806 | FALSE | TRUE | TRUE | TRUE | MISSENSE | FALSE |
| 5:96423320:G:A | 0.0000035 | FALSE | TRUE | FALSE | TRUE | MISSENSE | FALSE |
| 5:96423326:A:T | 0.0000169 | FALSE | TRUE | TRUE | TRUE | MISSENSE | FALSE |
| 5:96423332:G:A | 0.0000455 | FALSE | TRUE | TRUE | TRUE | MISSENSE | FALSE |
| 5:96423336:G:A | 0.0000105 | FALSE | TRUE | TRUE | TRUE | MISSENSE | FALSE |
| 5:96423337:A:T | 0.0000085 | FALSE | TRUE | TRUE | TRUE | MISSENSE | FALSE |
| 5:96423338:T:A | 0.0000085 | FALSE | TRUE | TRUE | TRUE | MISSENSE | FALSE |
| 5:96423354:CA:C | 0.0000065 | TRUE | TRUE | TRUE | TRUE | FRAMESHIFT | TRUE |
| 5:96423356:T:A | 0.0000085 | FALSE | TRUE | TRUE | TRUE | MISSENSE | FALSE |
| 5:96423363:C:T | 0.0000175 | FALSE | TRUE | FALSE | TRUE | MISSENSE | FALSE |
| 5:96423375:C:T | 0.0000035 | FALSE | TRUE | FALSE | TRUE | MISSENSE | FALSE |
| 5:96423377:C:T | 0.0000085 | FALSE | TRUE | TRUE | TRUE | MISSENSE | FALSE |
| 5:96423386:G:A | 0.0000070 | FALSE | TRUE | TRUE | TRUE | MISSENSE | FALSE |
| 5:96423387:T:A | 0.0000035 | FALSE | TRUE | FALSE | TRUE | MISSENSE | FALSE |
| 5:96423392:C:T | 0.0000065 | FALSE | TRUE | TRUE | TRUE | MISSENSE | FALSE |
| 5:96423393:C:T | 0.0000385 | FALSE | TRUE | TRUE | TRUE | MISSENSE | FALSE |
| 5:96423401:CAA:C | 0.0000035 | TRUE | TRUE | TRUE | TRUE | FRAMESHIFT | TRUE |
| 5:96423409:T:C | 0.0000065 | FALSE | TRUE | FALSE | TRUE | MISSENSE | FALSE |
| 5:96423410:A:G | 0.0000070 | FALSE | TRUE | FALSE | TRUE | MISSENSE | FALSE |
| 5:96423411:T:A | 0.0000105 | FALSE | TRUE | FALSE | TRUE | MISSENSE | FALSE |
| 5:96423416:T:G | 0.0000085 | FALSE | TRUE | TRUE | TRUE | MISSENSE | FALSE |
| 5:96423431:G:A | 0.0000065 | FALSE | TRUE | TRUE | TRUE | MISSENSE | FALSE |
| 5:96423443:G:A | 0.0000245 | FALSE | TRUE | FALSE | TRUE | MISSENSE | FALSE |
| 5:96423452:G:T | 0.0000245 | FALSE | TRUE | TRUE | TRUE | MISSENSE | FALSE |
| 5:96425819:C:G | 0.0000035 | TRUE | TRUE | TRUE | TRUE | SPLICE_DONOR | TRUE |
| 5:96425827:C:G | 0.0000035 | FALSE | TRUE | TRUE | TRUE | MISSENSE | FALSE |
| 5:96425827:C:T | 0.0000105 | TRUE | TRUE | TRUE | TRUE | STOP_GAINED | TRUE |
| 5:96425836:T:A | 0.0000070 | FALSE | TRUE | FALSE | TRUE | MISSENSE | FALSE |
| 5:96425836:T:C | 0.0000085 | FALSE | TRUE | FALSE | TRUE | MISSENSE | FALSE |
| 5:96425837:T:G | 0.0000175 | FALSE | TRUE | TRUE | TRUE | MISSENSE | FALSE |
| 5:96425841:C:T | 0.0002347 | FALSE | TRUE | FALSE | TRUE | MISSENSE | FALSE |
| 5:96425842:A:T | 0.0000175 | FALSE | TRUE | FALSE | TRUE | MISSENSE | FALSE |
| 5:96425843:TG:T | 0.0000130 | TRUE | TRUE | TRUE | TRUE | FRAMESHIFT | TRUE |
| 5:96425845:G:A | 0.0000065 | FALSE | TRUE | TRUE | TRUE | MISSENSE | FALSE |
| 5:96425851:T:C | 0.0000194 | FALSE | TRUE | FALSE | TRUE | MISSENSE | FALSE |
| 5:96425855:A:C | 0.0000175 | FALSE | TRUE | TRUE | TRUE | MISSENSE | FALSE |
| 5:96425881:G:A | 0.0000085 | FALSE | TRUE | FALSE | FALSE | MISSENSE | FALSE |
| 5:96425887:C:G | 0.0000065 | FALSE | TRUE | TRUE | TRUE | MISSENSE | FALSE |
| 5:96425887:C:T | 0.0000140 | FALSE | TRUE | TRUE | TRUE | MISSENSE | FALSE |
| 5:96425888:G:A | 0.0000140 | FALSE | TRUE | TRUE | TRUE | MISSENSE | FALSE |
| 5:96425891:T:A | 0.0000035 | TRUE | TRUE | TRUE | TRUE | STOP_GAINED | TRUE |
| 5:96425892:AC:A | 0.0000070 | TRUE | TRUE | TRUE | TRUE | FRAMESHIFT | TRUE |
| 5:96425893:C:G | 0.0000035 | FALSE | TRUE | FALSE | FALSE | MISSENSE | FALSE |
| 5:96425899:T:A | 0.0000035 | FALSE | TRUE | FALSE | TRUE | MISSENSE | FALSE |
| 5:96425920:G:A | 0.0000259 | FALSE | TRUE | FALSE | TRUE | MISSENSE | FALSE |
| 5:96425925:T:C | 0.0000070 | FALSE | TRUE | FALSE | FALSE | MISSENSE | FALSE |
| 5:96425929:A:G | 0.0000065 | FALSE | TRUE | TRUE | TRUE | MISSENSE | FALSE |
| 5:96429214:C:T | 0.0000035 | FALSE | TRUE | TRUE | TRUE | MISSENSE | FALSE |
| 5:96429215:G:A | 0.0000583 | FALSE | TRUE | TRUE | TRUE | MISSENSE | FALSE |
| 5:96429215:G:C | 0.0000035 | FALSE | TRUE | FALSE | TRUE | MISSENSE | FALSE |
| 5:96429217:T:C | 0.0000105 | FALSE | TRUE | FALSE | TRUE | MISSENSE | FALSE |
| 5:96429220:T:C | 0.0000070 | FALSE | TRUE | FALSE | TRUE | MISSENSE | FALSE |
| 5:96429228:T:A | 0.0000035 | FALSE | TRUE | TRUE | TRUE | MISSENSE | FALSE |
| 5:96429238:G:A | 0.0000085 | FALSE | TRUE | TRUE | TRUE | MISSENSE | FALSE |
| 5:96429242:T:C | 0.0000085 | FALSE | TRUE | FALSE | TRUE | MISSENSE | FALSE |
| 5:96429243:A:T | 0.0000035 | FALSE | TRUE | FALSE | TRUE | MISSENSE | FALSE |
| 5:96429244:T:C | 0.0000070 | FALSE | TRUE | FALSE | TRUE | MISSENSE | FALSE |
| 5:96429252:A:C | 0.0000035 | FALSE | TRUE | FALSE | TRUE | MISSENSE | FALSE |
| 5:96429253:C:G | 0.0000035 | FALSE | TRUE | FALSE | TRUE | MISSENSE | FALSE |
| 5:96429255:C:A | 0.0000035 | FALSE | TRUE | TRUE | TRUE | MISSENSE | FALSE |
| 5:96429256:CT:C | 0.0000065 | TRUE | TRUE | TRUE | TRUE | FRAMESHIFT | TRUE |
| 5:96429259:C:T | 0.0000065 | FALSE | TRUE | TRUE | TRUE | MISSENSE | FALSE |
| 5:96429260:G:A | 0.0000065 | TRUE | TRUE | TRUE | TRUE | STOP_GAINED | TRUE |
| 5:96429262:G:C | 0.0000245 | FALSE | TRUE | TRUE | TRUE | MISSENSE | FALSE |
| 5:96429264:C:A | 0.0000130 | FALSE | TRUE | FALSE | TRUE | MISSENSE | FALSE |
| 5:96429265:C:T | 0.0000085 | FALSE | TRUE | FALSE | TRUE | MISSENSE | FALSE |
| 5:96429269:TG:T | 0.0000130 | TRUE | TRUE | TRUE | TRUE | FRAMESHIFT | TRUE |
| 5:96429272:G:A | 0.0000035 | FALSE | TRUE | FALSE | TRUE | MISSENSE | FALSE |
| 5:96429283:T:C | 0.0000035 | FALSE | TRUE | FALSE | TRUE | MISSENSE | FALSE |
| 5:96429287:T:G | 0.0000035 | FALSE | TRUE | FALSE | TRUE | MISSENSE | FALSE |
| 5:96429290:A:G | 0.0000035 | FALSE | TRUE | FALSE | TRUE | MISSENSE | FALSE |
| 5:96429299:G:T | 0.0000070 | FALSE | TRUE | TRUE | TRUE | MISSENSE | FALSE |
| 5:96429310:G:A | 0.0000389 | FALSE | TRUE | FALSE | TRUE | MISSENSE | FALSE |
| 5:96429310:G:T | 0.0000070 | TRUE | TRUE | TRUE | TRUE | STOP_GAINED | TRUE |
| 5:96429319:T:C | 0.0000070 | TRUE | TRUE | TRUE | TRUE | SPLICE_ACCEPTOR | TRUE |
| 5:96432082:C:T | 0.0000035 | TRUE | TRUE | TRUE | TRUE | SPLICE_DONOR | TRUE |
| 5:96432094:A:G | 0.0000490 | FALSE | TRUE | FALSE | FALSE | MISSENSE | FALSE |
| 5:96432105:A:T | 0.0000140 | FALSE | TRUE | FALSE | TRUE | MISSENSE | FALSE |
| 5:96432106:T:C | 0.0000035 | FALSE | TRUE | FALSE | FALSE | MISSENSE | FALSE |
| 5:96432108:G:A | 0.0000035 | FALSE | TRUE | FALSE | TRUE | MISSENSE | FALSE |
| 5:96432109:A:G | 0.0000035 | FALSE | TRUE | FALSE | FALSE | MISSENSE | FALSE |
| 5:96432113:C:G | 0.0000194 | FALSE | TRUE | FALSE | TRUE | MISSENSE | FALSE |
| 5:96432115:T:C | 0.0000065 | FALSE | TRUE | FALSE | TRUE | MISSENSE | FALSE |
| 5:96432118:C:G | 0.0000065 | FALSE | TRUE | FALSE | TRUE | MISSENSE | FALSE |
| 5:96432861:A:C | 0.0000065 | TRUE | TRUE | TRUE | TRUE | SPLICE_DONOR | TRUE |
| 5:96432869:CA:C | 0.0000085 | TRUE | TRUE | TRUE | TRUE | FRAMESHIFT | TRUE |
| 5:96432870:A:G | 0.0000070 | FALSE | TRUE | FALSE | TRUE | MISSENSE | FALSE |
| 5:96432874:G:A | 0.0000070 | FALSE | TRUE | FALSE | TRUE | MISSENSE | FALSE |
| 5:96432876:T:C | 0.0000130 | FALSE | TRUE | FALSE | TRUE | MISSENSE | FALSE |
| 5:96432877:C:T | 0.0000035 | FALSE | TRUE | FALSE | TRUE | MISSENSE | FALSE |
| 5:96432886:G:C | 0.0000070 | FALSE | TRUE | FALSE | TRUE | MISSENSE | FALSE |
| 5:96432888:T:G | 0.0001166 | FALSE | TRUE | FALSE | TRUE | MISSENSE | FALSE |
| 5:96432889:C:T | 0.0000085 | FALSE | TRUE | FALSE | TRUE | MISSENSE | FALSE |
| 5:96432894:G:T | 0.0000085 | FALSE | TRUE | FALSE | TRUE | MISSENSE | FALSE |
| 5:96432895:C:A | 0.0000105 | FALSE | TRUE | FALSE | TRUE | MISSENSE | FALSE |
| 5:96432896:G:C | 0.0001878 | FALSE | TRUE | FALSE | TRUE | MISSENSE | FALSE |
| 5:96432897:A:T | 0.0000130 | FALSE | TRUE | FALSE | TRUE | MISSENSE | FALSE |
| 5:96432903:G:A | 0.0000140 | FALSE | TRUE | FALSE | FALSE | MISSENSE | FALSE |
| 5:96432909:G:T | 0.0000105 | FALSE | TRUE | FALSE | TRUE | MISSENSE | FALSE |
| 5:96432913:C:A | 0.0000035 | TRUE | TRUE | TRUE | TRUE | STOP_GAINED | TRUE |
| 5:96432915:G:C | 0.0000105 | FALSE | TRUE | FALSE | FALSE | MISSENSE | FALSE |
| 5:96432918:C:T | 0.0000490 | FALSE | TRUE | TRUE | TRUE | MISSENSE | FALSE |
| 5:96432921:C:G | 0.0000065 | FALSE | TRUE | FALSE | TRUE | MISSENSE | FALSE |
| 5:96432922:C:T | 0.0000140 | FALSE | TRUE | TRUE | TRUE | MISSENSE | FALSE |
| 5:96432927:A:G | 0.0000035 | FALSE | TRUE | FALSE | TRUE | MISSENSE | FALSE |
| 5:96432948:A:G | 0.0000085 | FALSE | TRUE | FALSE | TRUE | MISSENSE | FALSE |
| 5:96432952:A:G | 0.0000035 | FALSE | TRUE | FALSE | TRUE | MISSENSE | FALSE |
| 5:96432957:CT:C | 0.0000065 | TRUE | TRUE | TRUE | TRUE | FRAMESHIFT | TRUE |
| 5:96432961:T:C | 0.0000254 | FALSE | TRUE | FALSE | FALSE | MISSENSE | FALSE |
| 5:96432963:G:A | 0.0000911 | FALSE | TRUE | FALSE | FALSE | MISSENSE | FALSE |
| 5:96432965:T:G | 0.0000070 | FALSE | TRUE | FALSE | FALSE | MISSENSE | FALSE |
| 5:96432967:T:C | 0.0000596 | FALSE | TRUE | FALSE | FALSE | MISSENSE | FALSE |
| 5:96432967:T:G | 0.0001036 | FALSE | TRUE | FALSE | FALSE | MISSENSE | FALSE |
| 5:96432971:AC:A | 0.0000259 | TRUE | TRUE | TRUE | TRUE | FRAMESHIFT | TRUE |
| 5:96432972:C:T | 0.0000130 | FALSE | TRUE | FALSE | FALSE | MISSENSE | FALSE |
| 5:96432975:T:A | 0.0002720 | FALSE | TRUE | FALSE | FALSE | MISSENSE | FALSE |
| 5:96432975:T:C | 0.0000035 | FALSE | TRUE | FALSE | FALSE | MISSENSE | FALSE |
| 5:96432976:T:A | 0.0002720 | FALSE | TRUE | FALSE | FALSE | MISSENSE | FALSE |
| 5:96432978:A:G | 0.0000065 | FALSE | TRUE | FALSE | FALSE | MISSENSE | FALSE |
| 5:96432979:G:T | 0.0000035 | FALSE | TRUE | FALSE | FALSE | MISSENSE | FALSE |
| 5:96432981:G:GCA | 0.0000035 | TRUE | TRUE | TRUE | TRUE | FRAMESHIFT | TRUE |
| 5:96432981:G:T | 0.0000140 | FALSE | TRUE | FALSE | TRUE | MISSENSE | FALSE |
| 5:96432985:A:G | 0.0000070 | FALSE | TRUE | FALSE | TRUE | MISSENSE | FALSE |
| 5:96432991:C:A | 0.0000339 | FALSE | TRUE | FALSE | FALSE | MISSENSE | FALSE |
| 5:96432993:CAA:C | 0.0000065 | TRUE | TRUE | TRUE | TRUE | FRAMESHIFT | TRUE |
| 5:96433000:G:A | 0.0000070 | FALSE | TRUE | FALSE | FALSE | MISSENSE | FALSE |
| 5:96433004:G:T | 0.0000035 | FALSE | TRUE | FALSE | FALSE | MISSENSE | FALSE |
| 5:96433008:G:A | 0.0000065 | FALSE | TRUE | FALSE | TRUE | MISSENSE | FALSE |
| 5:96433016:C:G | 0.0000065 | FALSE | TRUE | FALSE | FALSE | MISSENSE | FALSE |
| 5:96433017:T:A | 0.0000070 | FALSE | TRUE | FALSE | FALSE | MISSENSE | FALSE |
| 5:96433017:T:C | 0.0000130 | FALSE | TRUE | FALSE | FALSE | MISSENSE | FALSE |
| 5:96433032:C:T | 0.0000065 | FALSE | TRUE | FALSE | FALSE | MISSENSE | FALSE |
| 5:96433040:C:T | 0.0000065 | TRUE | TRUE | TRUE | TRUE | START_LOST | TRUE |
| 5:96433041:A:G | 0.0000035 | TRUE | TRUE | TRUE | TRUE | START_LOST | TRUE |

TABLE 5

| VARIANT | AAF | M1 | M2 | M3 | M4 | ANNOTATION | IS_LOF |
|---|---|---|---|---|---|---|---|
| 5:96393044:C:T | 0.0000245 | FALSE | TRUE | TRUE | TRUE | MISSENSE | FALSE |
| 5:96393045:G:A | 0.0002452 | FALSE | TRUE | TRUE | TRUE | MISSENSE | FALSE |
| 5:96393048:C:T | 0.0000420 | FALSE | TRUE | TRUE | TRUE | MISSENSE | FALSE |
| 5:96393050:T:C | 0.0000070 | FALSE | TRUE | TRUE | TRUE | MISSENSE | FALSE |
| 5:96393053:C:T | 0.0000035 | FALSE | TRUE | TRUE | TRUE | MISSENSE | FALSE |
| 5:96393057:G:T | 0.0000065 | FALSE | TRUE | TRUE | TRUE | MISSENSE | FALSE |
| 5:96393065:G:A | 0.0000194 | FALSE | TRUE | TRUE | TRUE | MISSENSE | FALSE |
| 5:96393077:T:C | 0.0000035 | FALSE | TRUE | TRUE | TRUE | MISSENSE | FALSE |
| 5:96393078:A:G | 0.0000035 | FALSE | TRUE | TRUE | TRUE | MISSENSE | FALSE |
| 5:96393101:T:C | 0.0000070 | FALSE | TRUE | TRUE | TRUE | MISSENSE | FALSE |
| 5:96393104:A:C | 0.0000035 | FALSE | TRUE | TRUE | TRUE | MISSENSE | FALSE |
| 5:96393105:G:T | 0.0000035 | FALSE | TRUE | TRUE | TRUE | MISSENSE | FALSE |
| 5:96393155:G:A | 0.0000035 | FALSE | TRUE | TRUE | TRUE | MISSENSE | FALSE |
| 5:96393156:C:T | 0.0000035 | FALSE | TRUE | TRUE | TRUE | MISSENSE | FALSE |
| 5:96393172:A:C | 0.0000085 | TRUE | TRUE | TRUE | TRUE | STOP_GAINED | TRUE |
| 5:96393239:A:G | 0.0000140 | FALSE | TRUE | TRUE | TRUE | MISSENSE | FALSE |
| 5:96393258:G:A | 0.0000070 | TRUE | TRUE | TRUE | TRUE | STOP_GAINED | TRUE |
| 5:96393366:G:A | 0.0000194 | TRUE | TRUE | TRUE | TRUE | STOP_GAINED | TRUE |
| 5:96394925:G:A | 0.0000175 | FALSE | TRUE | TRUE | TRUE | MISSENSE | FALSE |
| 5:96394934:C:T | 0.0000175 | FALSE | TRUE | TRUE | TRUE | MISSENSE | FALSE |
| 5:96394935:G:A | 0.0000035 | FALSE | TRUE | TRUE | TRUE | MISSENSE | FALSE |
| 5:96394941:G:A | 0.0000035 | TRUE | TRUE | TRUE | TRUE | STOP_GAINED | TRUE |
| 5:96394973:T:C | 0.0000035 | FALSE | TRUE | TRUE | TRUE | MISSENSE | FALSE |
| 5:96394988:C:T | 0.0000065 | TRUE | TRUE | TRUE | TRUE | STOP_GAINED | TRUE |
| 5:96394997:A:G | 0.0000035 | FALSE | TRUE | TRUE | TRUE | MISSENSE | FALSE |
| 5:96395003:C:T | 0.0000065 | FALSE | TRUE | TRUE | TRUE | MISSENSE | FALSE |
| 5:96395024:G:C | 0.0000065 | FALSE | TRUE | TRUE | TRUE | MISSENSE | FALSE |
| 5:96397346:A:T | 0.0000105 | FALSE | TRUE | TRUE | TRUE | MISSENSE | FALSE |
| 5:96397351:CAA:C | 0.0000194 | TRUE | TRUE | TRUE | TRUE | FRAMESHIFT | TRUE |
| 5:96397351:C:G | 0.0000254 | FALSE | TRUE | TRUE | TRUE | MISSENSE | FALSE |
| 5:96397357:C:G | 0.0000065 | FALSE | TRUE | TRUE | TRUE | MISSENSE | FALSE |
| 5:96397358:C:T | 0.0000085 | TRUE | TRUE | TRUE | TRUE | STOP_GAINED | TRUE |
| 5:96397364:C:A | 0.0000065 | FALSE | TRUE | TRUE | TRUE | MISSENSE | FALSE |
| 5:96397377:C:A | 0.0000065 | TRUE | TRUE | TRUE | TRUE | STOP_GAINED | TRUE |
| 5:96397380:C:T | 0.0000065 | FALSE | TRUE | TRUE | TRUE | MISSENSE | FALSE |
| 5:96397382:C:T | 0.0000035 | TRUE | TRUE | TRUE | TRUE | STOP_GAINED | TRUE |
| 5:96397390:AAC:A | 0.0000065 | TRUE | TRUE | TRUE | TRUE | FRAMESHIFT | TRUE |
| 5:96397397:A:G | 0.0000065 | FALSE | TRUE | TRUE | TRUE | MISSENSE | FALSE |
| 5:96397432:A:T | 0.0000035 | FALSE | TRUE | TRUE | TRUE | MISSENSE | FALSE |
| 5:96397433:T:C | 0.0000070 | FALSE | TRUE | TRUE | TRUE | MISSENSE | FALSE |
| 5:96397440:C:A | 0.0000065 | TRUE | TRUE | TRUE | TRUE | STOP_GAINED | TRUE |
| 5:96397451:A:G | 0.0000035 | FALSE | TRUE | TRUE | TRUE | MISSENSE | FALSE |
| 5:96398877:A:G | 0.0000065 | TRUE | TRUE | TRUE | TRUE | SPLICE_DONOR | TRUE |
| 5:96398887:GAAGT:G | 0.0000085 | TRUE | TRUE | TRUE | TRUE | FRAMESHIFT | TRUE |
| 5:96398911:C:A | 0.0000130 | FALSE | TRUE | TRUE | TRUE | MISSENSE | FALSE |
| 5:96398917:C:T | 0.0002172 | FALSE | TRUE | TRUE | TRUE | MISSENSE | FALSE |
| 5:96398922:A:C | 0.0000035 | TRUE | TRUE | TRUE | TRUE | STOP_GAINED | TRUE |
| 5:96398929:A:G | 0.0000035 | FALSE | TRUE | TRUE | TRUE | MISSENSE | FALSE |
| 5:96399952:C:A | 0.0000065 | TRUE | TRUE | TRUE | TRUE | SPLICE_DONOR | TRUE |
| 5:96399993:T:A | 0.0000105 | TRUE | TRUE | TRUE | TRUE | STOP_GAINED | TRUE |
| 5:96399999:G:T | 0.0000175 | FALSE | TRUE | TRUE | TRUE | MISSENSE | FALSE |
| 5:96400002:C:T | 0.0000596 | FALSE | TRUE | TRUE | TRUE | MISSENSE | FALSE |
| 5:96400010:C:G | 0.0000085 | FALSE | TRUE | TRUE | TRUE | MISSENSE | FALSE |
| 5:96400028:A:G | 0.0000065 | FALSE | TRUE | TRUE | TRUE | MISSENSE | FALSE |
| 5:96400038:G:C | 0.0000070 | FALSE | TRUE | TRUE | TRUE | MISSENSE | FALSE |
| 5:96400047:C:A | 0.0000035 | FALSE | TRUE | TRUE | TRUE | MISSENSE | FALSE |
| 5:96400049:T:A | 0.0000035 | FALSE | TRUE | TRUE | TRUE | MISSENSE | FALSE |
| 5:96400052:A:G | 0.0000065 | FALSE | TRUE | TRUE | TRUE | MISSENSE | FALSE |
| 5:96400071:G:A | 0.0000070 | TRUE | TRUE | TRUE | TRUE | STOP_GAINED | TRUE |
| 5:96400091:G:T | 0.0000035 | FALSE | TRUE | TRUE | TRUE | MISSENSE | FALSE |
| 5:96400096:A:T | 0.0000259 | FALSE | TRUE | TRUE | TRUE | MISSENSE | FALSE |
| 5:96400106:C:T | 0.0000130 | TRUE | TRUE | TRUE | TRUE | STOP_GAINED | TRUE |
| 5:96400156:G:C | 0.0000065 | FALSE | TRUE | TRUE | TRUE | MISSENSE | FALSE |
| 5:96400169:C:T | 0.0000035 | FALSE | TRUE | TRUE | TRUE | MISSENSE | FALSE |
| 5:96400170:G:A | 0.0000175 | TRUE | TRUE | TRUE | TRUE | STOP_GAINED | TRUE |
| 5:96408221:A:C | 0.0000035 | TRUE | TRUE | TRUE | TRUE | SPLICE_DONOR | TRUE |
| 5:96408226:G:T | 0.0000280 | FALSE | TRUE | TRUE | TRUE | MISSENSE | FALSE |
| 5:96408232:A:G | 0.0000130 | FALSE | TRUE | TRUE | TRUE | MISSENSE | FALSE |
| 5:96408238:A:G | 0.0000035 | FALSE | TRUE | TRUE | TRUE | MISSENSE | FALSE |
| 5:96408242:C:G | 0.0000065 | FALSE | TRUE | TRUE | TRUE | MISSENSE | FALSE |
| 5:96408242:C:T | 0.0000070 | FALSE | TRUE | TRUE | TRUE | MISSENSE | FALSE |
| 5:96408244:A:G | 0.0000280 | FALSE | TRUE | TRUE | TRUE | MISSENSE | FALSE |
| 5:96408251:C:T | 0.0000035 | FALSE | TRUE | TRUE | TRUE | MISSENSE | FALSE |
| 5:96408254:C:T | 0.0000035 | FALSE | TRUE | TRUE | TRUE | MISSENSE | FALSE |
| 5:96408256:G:T | 0.0000065 | FALSE | TRUE | TRUE | TRUE | MISSENSE | FALSE |
| 5:96408272:C:T | 0.0000065 | FALSE | TRUE | TRUE | TRUE | MISSENSE | FALSE |
| 5:96408274:G:A | 0.0000105 | FALSE | TRUE | TRUE | TRUE | MISSENSE | FALSE |
| 5:96408283:G:C | 0.0000194 | FALSE | TRUE | TRUE | TRUE | MISSENSE | FALSE |
| 5:96408285:G:T | 0.0000085 | FALSE | TRUE | TRUE | TRUE | MISSENSE | FALSE |
| 5:96408288:C:CA | 0.0000065 | TRUE | TRUE | TRUE | TRUE | FRAMESHIFT | TRUE |
| 5:96408295:G:A | 0.0000035 | FALSE | TRUE | TRUE | TRUE | MISSENSE | FALSE |
| 5:96408296:T:A | 0.0000065 | FALSE | TRUE | TRUE | TRUE | MISSENSE | FALSE |
| 5:96408299:A:AG | 0.0000065 | TRUE | TRUE | TRUE | TRUE | FRAMESHIFT | TRUE |
| 5:96408310:A:AG | 0.0000035 | TRUE | TRUE | TRUE | TRUE | FRAMESHIFT | TRUE |
| 5:96408312:G:C | 0.0000085 | FALSE | TRUE | TRUE | TRUE | MISSENSE | FALSE |
| 5:96408313:T:C | 0.0000065 | FALSE | TRUE | TRUE | TRUE | MISSENSE | FALSE |
| 5:96408324:C:G | 0.0000035 | TRUE | TRUE | TRUE | TRUE | SPLICE_ACCEPTOR | TRUE |
| 5:96410771:T:TA | 0.0000105 | TRUE | TRUE | TRUE | TRUE | SPLICE_DONOR | TRUE |
| 5:96410773:C:T | 0.0000065 | TRUE | TRUE | TRUE | TRUE | SPLICE_DONOR | TRUE |
| 5:96410785:C:T | 0.0000035 | FALSE | TRUE | TRUE | TRUE | MISSENSE | FALSE |
| 5:96410797:C:A | 0.0000070 | TRUE | TRUE | TRUE | TRUE | STOP_GAINED | TRUE |
| 5:96410797:C:T | 0.0000035 | FALSE | TRUE | TRUE | TRUE | MISSENSE | FALSE |
| 5:96410808:G:C | 0.0000035 | FALSE | TRUE | TRUE | TRUE | MISSENSE | FALSE |
| 5:96410814:G:A | 0.0000035 | FALSE | TRUE | TRUE | TRUE | MISSENSE | FALSE |
| 5:96410818:G:T | 0.0000065 | FALSE | TRUE | TRUE | TRUE | MISSENSE | FALSE |
| 5:96410826:G:A | 0.0000035 | FALSE | TRUE | TRUE | TRUE | MISSENSE | FALSE |
| 5:96410840:G:C | 0.0000280 | TRUE | TRUE | TRUE | TRUE | STOP_GAINED | TRUE |
| 5:96410840:G:T | 0.0000065 | TRUE | TRUE | TRUE | TRUE | STOP_GAINED | TRUE |
| 5:96410842:A:G | 0.0000085 | FALSE | TRUE | TRUE | TRUE | MISSENSE | FALSE |
| 5:96410844:CAGGGGGAT:C | 0.0000070 | TRUE | TRUE | TRUE | TRUE | FRAMESHIFT | TRUE |
| 5:96410845:A:C | 0.0000210 | FALSE | TRUE | TRUE | TRUE | MISSENSE | FALSE |
| 5:96410856:C:A | 0.0000140 | FALSE | TRUE | TRUE | TRUE | MISSENSE | FALSE |
| 5:96410856:C:T | 0.0000070 | FALSE | TRUE | TRUE | TRUE | MISSENSE | FALSE |
| 5:96410857:C:A | 0.0000065 | FALSE | TRUE | TRUE | TRUE | MISSENSE | FALSE |
| 5:96410878:T:A | 0.0000070 | FALSE | TRUE | TRUE | TRUE | MISSENSE | FALSE |
| 5:96410883:A:G | 0.0000105 | FALSE | TRUE | TRUE | TRUE | MISSENSE | FALSE |
| 5:96410887:T:C | 0.0000065 | FALSE | TRUE | TRUE | TRUE | MISSENSE | FALSE |
| 5:96410895:C:A | 0.0000065 | FALSE | TRUE | TRUE | TRUE | MISSENSE | FALSE |
| 5:96410901:G:A | 0.0000035 | FALSE | TRUE | TRUE | TRUE | MISSENSE | FALSE |
| 5:96410907:C:A | 0.0000035 | FALSE | TRUE | TRUE | TRUE | MISSENSE | FALSE |
| 5:96410907:C:T | 0.0000035 | FALSE | TRUE | TRUE | TRUE | MISSENSE | FALSE |
| 5:96410910:T:TC | 0.0000130 | TRUE | TRUE | TRUE | TRUE | FRAMESHIFT | TRUE |
| 5:96410913:C:T | 0.0000065 | FALSE | TRUE | TRUE | TRUE | MISSENSE | FALSE |
| 5:96410935:G:A | 0.0000035 | FALSE | TRUE | TRUE | TRUE | MISSENSE | FALSE |
| 5:96410935:G:GC | 0.0000035 | TRUE | TRUE | TRUE | TRUE | FRAMESHIFT | TRUE |
| 5:96410940:C:A | 0.0000035 | FALSE | TRUE | TRUE | TRUE | MISSENSE | FALSE |
| 5:96410941:C:T | 0.0000085 | FALSE | TRUE | TRUE | TRUE | MISSENSE | FALSE |
| 5:96410946:C:T | 0.0000105 | FALSE | TRUE | TRUE | TRUE | MISSENSE | FALSE |
| 5:96410970:C:T | 0.0000035 | FALSE | TRUE | TRUE | TRUE | MISSENSE | FALSE |
| 5:96410974:TC:T | 0.0000712 | TRUE | TRUE | TRUE | TRUE | FRAMESHIFT | TRUE |
| 5:96410976:C:G | 0.0000065 | FALSE | TRUE | TRUE | TRUE | MISSENSE | FALSE |
| 5:96410983:T:TC | 0.0000035 | TRUE | TRUE | TRUE | TRUE | FRAMESHIFT | TRUE |
| 5:96410985:C:T | 0.0000065 | FALSE | TRUE | TRUE | TRUE | MISSENSE | FALSE |
| 5:96412328:C:T | 0.0000035 | FALSE | TRUE | TRUE | TRUE | MISSENSE | FALSE |
| 5:96412356:G:A | 0.0000175 | FALSE | TRUE | TRUE | TRUE | MISSENSE | FALSE |
| 5:96412362:G:A | 0.0000130 | FALSE | TRUE | TRUE | TRUE | MISSENSE | FALSE |
| 5:96412362:G:T | 0.0000035 | FALSE | TRUE | TRUE | TRUE | MISSENSE | FALSE |
| 5:96412364:C:G | 0.0000169 | FALSE | TRUE | TRUE | TRUE | MISSENSE | FALSE |
| 5:96412379:C:G | 0.0000070 | FALSE | TRUE | TRUE | TRUE | MISSENSE | FALSE |
| 5:96412382:T:C | 0.0000065 | FALSE | TRUE | TRUE | TRUE | MISSENSE | FALSE |
| 5:96412385:T:C | 0.0000169 | FALSE | TRUE | TRUE | TRUE | MISSENSE | FALSE |
| 5:96412395:C:T | 0.0000065 | FALSE | TRUE | TRUE | TRUE | MISSENSE | FALSE |
| 5:96412400:C:T | 0.0000085 | FALSE | TRUE | TRUE | TRUE | MISSENSE | FALSE |
| 5:96412416:C:T | 0.0000130 | FALSE | TRUE | TRUE | TRUE | MISSENSE | FALSE |
| 5:96412418:A:G | 0.0000140 | FALSE | TRUE | TRUE | TRUE | MISSENSE | FALSE |
| 5:96412419:C:T | 0.0000070 | FALSE | TRUE | TRUE | TRUE | MISSENSE | FALSE |
| 5:96412420:G:T | 0.0000105 | FALSE | TRUE | TRUE | TRUE | MISSENSE | FALSE |
| 5:96412436:C:G | 0.0000065 | FALSE | TRUE | TRUE | TRUE | MISSENSE | FALSE |
| 5:96412436:C:T | 0.0000070 | FALSE | TRUE | TRUE | TRUE | MISSENSE | FALSE |
| 5:96412437:C:CA | 0.0000065 | TRUE | TRUE | TRUE | TRUE | FRAMESHIFT | TRUE |
| 5:96412437:C:T | 0.0000035 | FALSE | TRUE | TRUE | TRUE | MISSENSE | FALSE |
| 5:96412439:A:G | 0.0000085 | FALSE | TRUE | TRUE | TRUE | MISSENSE | FALSE |
| 5:96412443:A:T | 0.0000065 | FALSE | TRUE | TRUE | TRUE | MISSENSE | FALSE |
| 5:96412448:G:A | 0.0000070 | FALSE | TRUE | TRUE | TRUE | MISSENSE | FALSE |
| 5:96412452:C:A | 0.0000105 | TRUE | TRUE | TRUE | TRUE | STOP_GAINED | TRUE |
| 5:96412454:A:AT | 0.0000065 | TRUE | TRUE | TRUE | TRUE | FRAMESHIFT | TRUE |
| 5:96412454:A:G | 0.0000035 | FALSE | TRUE | TRUE | TRUE | MISSENSE | FALSE |
| 5:96412463:G:A | 0.0000140 | FALSE | TRUE | TRUE | TRUE | MISSENSE | FALSE |
| 5:96412464:T:A | 0.0000035 | FALSE | TRUE | TRUE | TRUE | MISSENSE | FALSE |
| 5:96412480:C:G | 0.0000035 | FALSE | TRUE | TRUE | TRUE | MISSENSE | FALSE |
| 5:96412484:C:A | 0.0000085 | FALSE | TRUE | TRUE | TRUE | MISSENSE | FALSE |
| 5:96416041:T:A | 0.0001296 | FALSE | TRUE | TRUE | TRUE | MISSENSE | FALSE |
| 5:96416041:T:G | 0.0000065 | FALSE | TRUE | TRUE | TRUE | MISSENSE | FALSE |
| 5:96416050:T:C | 0.0000130 | FALSE | TRUE | TRUE | TRUE | MISSENSE | FALSE |
| 5:96416051:A:G | 0.0000070 | FALSE | TRUE | TRUE | TRUE | MISSENSE | FALSE |
| 5:96416053:G:A | 0.0000194 | FALSE | TRUE | TRUE | TRUE | MISSENSE | FALSE |
| 5:96416054:C:A | 0.0000035 | FALSE | TRUE | TRUE | TRUE | MISSENSE | FALSE |
| 5:96416054:C:G | 0.0000065 | FALSE | TRUE | TRUE | TRUE | MISSENSE | FALSE |
| 5:96416059:C:A | 0.0000105 | FALSE | TRUE | TRUE | TRUE | MISSENSE | FALSE |
| 5:96416063:C:T | 0.0000070 | FALSE | TRUE | TRUE | TRUE | MISSENSE | FALSE |
| 5:96416066:C:G | 0.0000035 | FALSE | TRUE | TRUE | TRUE | MISSENSE | FALSE |
| 5:96416066:C:T | 0.0000210 | FALSE | TRUE | TRUE | TRUE | MISSENSE | FALSE |
| 5:96416067:G:T | 0.0000035 | TRUE | TRUE | TRUE | TRUE | STOP_GAINED | TRUE |
| 5:96416068:C:T | 0.0000035 | FALSE | TRUE | TRUE | TRUE | MISSENSE | FALSE |
| 5:96416069:A:G | 0.0000035 | FALSE | TRUE | TRUE | TRUE | MISSENSE | FALSE |
| 5:96416072:T:A | 0.0000035 | TRUE | TRUE | TRUE | TRUE | STOP_GAINED | TRUE |
| 5:96416075:G:A | 0.0000035 | FALSE | TRUE | FALSE | TRUE | MISSENSE | FALSE |
| 5:96416092:G:A | 0.0001787 | FALSE | TRUE | TRUE | TRUE | MISSENSE | FALSE |
| 5:96416105:C:A | 0.0000035 | FALSE | TRUE | TRUE | TRUE | MISSENSE | FALSE |
| 5:96416105:C:T | 0.0000035 | FALSE | TRUE | TRUE | TRUE | MISSENSE | FALSE |
| 5:96416117:C:T | 0.0000035 | FALSE | TRUE | TRUE | TRUE | MISSENSE | FALSE |
| 5:96421879:CT:C | 0.0000035 | TRUE | TRUE | TRUE | TRUE | FRAMESHIFT | TRUE |
| 5:96421881:T:A | 0.0000035 | TRUE | TRUE | TRUE | TRUE | STOP_GAINED | TRUE |
| 5:96421882:G:T | 0.0000105 | FALSE | TRUE | TRUE | TRUE | MISSENSE | FALSE |
| 5:96421887:C:T | 0.0000105 | FALSE | TRUE | FALSE | TRUE | MISSENSE | FALSE |
| 5:96421893:TG:T | 0.0000065 | TRUE | TRUE | TRUE | TRUE | FRAMESHIFT | TRUE |
| 5:96421896:G:A | 0.0000070 | FALSE | TRUE | FALSE | TRUE | MISSENSE | FALSE |
| 5:96421898:T:A | 0.0000070 | FALSE | TRUE | TRUE | TRUE | MISSENSE | FALSE |
| 5:96421902:A:T | 0.0000035 | FALSE | TRUE | TRUE | TRUE | MISSENSE | FALSE |
| 5:96421904:C:T | 0.0000105 | FALSE | TRUE | TRUE | TRUE | MISSENSE | FALSE |
| 5:96421905:G:A | 0.0000194 | TRUE | TRUE | TRUE | TRUE | STOP_GAINED | TRUE |
| 5:96421908:G:A | 0.0000065 | FALSE | TRUE | TRUE | TRUE | MISSENSE | FALSE |
| 5:96421914:G:A | 0.0000194 | FALSE | TRUE | TRUE | TRUE | MISSENSE | FALSE |
| 5:96421916:T:C | 0.0000070 | FALSE | TRUE | TRUE | TRUE | MISSENSE | FALSE |
| 5:96421916:T:G | 0.0000105 | FALSE | TRUE | TRUE | TRUE | MISSENSE | FALSE |
| 5:96421922:T:C | 0.0000035 | FALSE | TRUE | TRUE | TRUE | MISSENSE | FALSE |
| 5:96421932:T:A | 0.0000065 | FALSE | TRUE | TRUE | TRUE | MISSENSE | FALSE |
| 5:96421940:TA:T | 0.0000065 | TRUE | TRUE | TRUE | TRUE | FRAMESHIFT | TRUE |
| 5:96421941:A:G | 0.0000350 | FALSE | TRUE | TRUE | TRUE | MISSENSE | FALSE |
| 5:96421946:G:C | 0.0000210 | FALSE | TRUE | TRUE | TRUE | MISSENSE | FALSE |
| 5:96421950:C:G | 0.0000035 | FALSE | TRUE | FALSE | TRUE | MISSENSE | FALSE |
| 5:96421951:TG:T | 0.0000065 | TRUE | TRUE | TRUE | TRUE | FRAMESHIFT | TRUE |
| 5:96421954:AT:A | 0.0000065 | TRUE | TRUE | TRUE | TRUE | FRAMESHIFT | TRUE |
| 5:96423315:A:G | 0.0003818 | FALSE | TRUE | TRUE | TRUE | MISSENSE | FALSE |
| 5:96423317:T:C | 0.0000806 | FALSE | TRUE | TRUE | TRUE | MISSENSE | FALSE |
| 5:96423326:A:T | 0.0000169 | FALSE | TRUE | TRUE | TRUE | MISSENSE | FALSE |
| 5:96423332:G:A | 0.0000455 | FALSE | TRUE | TRUE | TRUE | MISSENSE | FALSE |
| 5:96423336:G:A | 0.0000105 | FALSE | TRUE | TRUE | TRUE | MISSENSE | FALSE |
| 5:96423337:A:T | 0.0000085 | FALSE | TRUE | TRUE | TRUE | MISSENSE | FALSE |
| 5:96423338:T:A | 0.0000085 | FALSE | TRUE | TRUE | TRUE | MISSENSE | FALSE |
| 5:96423354:CA:C | 0.0000065 | TRUE | TRUE | TRUE | TRUE | FRAMESHIFT | TRUE |
| 5:96423356:T:A | 0.0000085 | FALSE | TRUE | TRUE | TRUE | MISSENSE | FALSE |
| 5:96423377:C:T | 0.0000085 | FALSE | TRUE | TRUE | TRUE | MISSENSE | FALSE |
| 5:96423386:G:A | 0.0000070 | FALSE | TRUE | TRUE | TRUE | MISSENSE | FALSE |
| 5:96423392:C:T | 0.0000065 | FALSE | TRUE | TRUE | TRUE | MISSENSE | FALSE |
| 5:96423393:C:T | 0.0000385 | FALSE | TRUE | TRUE | TRUE | MISSENSE | FALSE |
| 5:96423401:CAA:C | 0.0000035 | TRUE | TRUE | TRUE | TRUE | FRAMESHIFT | TRUE |
| 5:96423416:T:G | 0.0000085 | FALSE | TRUE | TRUE | TRUE | MISSENSE | FALSE |
| 5:96423431:G:A | 0.0000065 | FALSE | TRUE | TRUE | TRUE | MISSENSE | FALSE |
| 5:96423452:G:T | 0.0000245 | FALSE | TRUE | TRUE | TRUE | MISSENSE | FALSE |
| 5:96425819:C:G | 0.0000035 | TRUE | TRUE | TRUE | TRUE | SPLICE_DONOR | TRUE |
| 5:96425827:C:G | 0.0000035 | FALSE | TRUE | TRUE | TRUE | MISSENSE | FALSE |
| 5:96425827:C:T | 0.0000105 | TRUE | TRUE | TRUE | TRUE | STOP_GAINED | TRUE |
| 5:96425837:T:G | 0.0000175 | FALSE | TRUE | TRUE | TRUE | MISSENSE | FALSE |
| 5:96425843:TG:T | 0.0000130 | TRUE | TRUE | TRUE | TRUE | FRAMESHIFT | TRUE |
| 5:96425845:G:A | 0.0000065 | FALSE | TRUE | TRUE | TRUE | MISSENSE | FALSE |
| 5:96425855:A:C | 0.0000175 | FALSE | TRUE | TRUE | TRUE | MISSENSE | FALSE |
| 5:96425887:C:G | 0.0000065 | FALSE | TRUE | TRUE | TRUE | MISSENSE | FALSE |
| 5:96425887:C:T | 0.0000140 | FALSE | TRUE | TRUE | TRUE | MISSENSE | FALSE |
| 5:96425888:G:A | 0.0000140 | FALSE | TRUE | TRUE | TRUE | MISSENSE | FALSE |
| 5:96425891:T:A | 0.0000035 | TRUE | TRUE | TRUE | TRUE | STOP_GAINED | TRUE |
| 5:96425892:AC:A | 0.0000070 | TRUE | TRUE | TRUE | TRUE | FRAMESHIFT | TRUE |
| 5:96425929:A:G | 0.0000065 | FALSE | TRUE | TRUE | TRUE | MISSENSE | FALSE |
| 5:96429214:C:T | 0.0000035 | FALSE | TRUE | TRUE | TRUE | MISSENSE | FALSE |
| 5:96429215:G:A | 0.0000583 | FALSE | TRUE | TRUE | TRUE | MISSENSE | FALSE |
| 5:96429228:T:A | 0.0000035 | FALSE | TRUE | TRUE | TRUE | MISSENSE | FALSE |
| 5:96429238:G:A | 0.0000085 | FALSE | TRUE | TRUE | TRUE | MISSENSE | FALSE |
| 5:96429255:C:A | 0.0000035 | FALSE | TRUE | TRUE | TRUE | MISSENSE | FALSE |
| 5:96429256:CT:C | 0.0000065 | TRUE | TRUE | TRUE | TRUE | FRAMESHIFT | TRUE |
| 5:96429259:C:T | 0.0000065 | FALSE | TRUE | TRUE | TRUE | MISSENSE | FALSE |
| 5:96429260:G:A | 0.0000065 | TRUE | TRUE | TRUE | TRUE | STOP_GAINED | TRUE |
| 5:96429262:G:C | 0.0000245 | FALSE | TRUE | TRUE | TRUE | MISSENSE | FALSE |
| 5:96429269:TG:T | 0.0000130 | TRUE | TRUE | TRUE | TRUE | FRAMESHIFT | TRUE |
| 5:96429299:G:T | 0.0000070 | FALSE | TRUE | TRUE | TRUE | MISSENSE | FALSE |
| 5:96429310:G:T | 0.0000070 | TRUE | TRUE | TRUE | TRUE | STOP_GAINED | TRUE |
| 5:96429319:T:C | 0.0000070 | TRUE | TRUE | TRUE | TRUE | SPLICE_ACCEPTOR | TRUE |
| 5:96432082:C:T | 0.0000035 | TRUE | TRUE | TRUE | TRUE | SPLICE_DONOR | TRUE |
| 5:96432861:A:C | 0.0000065 | TRUE | TRUE | TRUE | TRUE | SPLICE_DONOR | TRUE |
| 5:96432869:CA:C | 0.0000085 | TRUE | TRUE | TRUE | TRUE | FRAMESHIFT | TRUE |
| 5:96432913:C:A | 0.0000035 | TRUE | TRUE | TRUE | TRUE | STOP_GAINED | TRUE |
| 5:96432918:C:T | 0.0000490 | FALSE | TRUE | TRUE | TRUE | MISSENSE | FALSE |
| 5:96432922:C:T | 0.0000140 | FALSE | TRUE | TRUE | TRUE | MISSENSE | FALSE |
| 5:96432957:CT:C | 0.0000065 | TRUE | TRUE | TRUE | TRUE | FRAMESHIFT | TRUE |
| 5:96432971:AC:A | 0.0000259 | TRUE | TRUE | TRUE | TRUE | FRAMESHIFT | TRUE |
| 5:96432981:G:GCA | 0.0000035 | TRUE | TRUE | TRUE | TRUE | FRAMESHIFT | TRUE |
| 5:96432993:CAA:C | 0.0000065 | TRUE | TRUE | TRUE | TRUE | FRAMESHIFT | TRUE |
| 5:96433040:C:T | 0.0000065 | TRUE | TRUE | TRUE | TRUE | START_LOST | TRUE |
| 5:96433041:A:G | 0.0000035 | TRUE | TRUE | TRUE | TRUE | START_LOST | TRUE |

TABLE 6

| VARIANT | AAF | M1 | M2 | M3 | M4 | ANNOTATION | IS_LOF |
|---|---|---|---|---|---|---|---|
| 5:96393012:C:T | 0.0000175 | FALSE | TRUE | FALSE | TRUE | MISSENSE | FALSE |
| 5:96393026:A:C | 0.0000035 | FALSE | TRUE | FALSE | TRUE | MISSENSE | FALSE |
| 5:96393027:C:T | 0.0000175 | FALSE | TRUE | FALSE | TRUE | MISSENSE | FALSE |
| 5:96393044:C:T | 0.0000245 | FALSE | TRUE | TRUE | TRUE | MISSENSE | FALSE |
| 5:96393045:G:A | 0.0002452 | FALSE | TRUE | TRUE | TRUE | MISSENSE | FALSE |
| 5:96393048:C:T | 0.0000420 | FALSE | TRUE | TRUE | TRUE | MISSENSE | FALSE |
| 5:96393050:T:C | 0.0000070 | FALSE | TRUE | TRUE | TRUE | MISSENSE | FALSE |
| 5:96393053:C:T | 0.0000035 | FALSE | TRUE | TRUE | TRUE | MISSENSE | FALSE |
| 5:96393057:G:T | 0.0000065 | FALSE | TRUE | TRUE | TRUE | MISSENSE | FALSE |
| 5:96393065:G:A | 0.0000194 | FALSE | TRUE | TRUE | TRUE | MISSENSE | FALSE |
| 5:96393077:T:C | 0.0000035 | FALSE | TRUE | TRUE | TRUE | MISSENSE | FALSE |
| 5:96393078:A:G | 0.0000035 | FALSE | TRUE | TRUE | TRUE | MISSENSE | FALSE |
| 5:96393083:A:C | 0.0000035 | FALSE | TRUE | FALSE | TRUE | MISSENSE | FALSE |
| 5:96393101:T:C | 0.0000070 | FALSE | TRUE | TRUE | TRUE | MISSENSE | FALSE |
| 5:96393104:A:C | 0.0000035 | FALSE | TRUE | TRUE | TRUE | MISSENSE | FALSE |
| 5:96393105:G:C | 0.0000035 | FALSE | TRUE | FALSE | TRUE | MISSENSE | FALSE |
| 5:96393105:G:T | 0.0000035 | FALSE | TRUE | TRUE | TRUE | MISSENSE | FALSE |
| 5:96393134:G:C | 0.0000085 | FALSE | TRUE | FALSE | TRUE | MISSENSE | FALSE |
| 5:96393155:G:A | 0.0000035 | FALSE | TRUE | TRUE | TRUE | MISSENSE | FALSE |
| 5:96393156:C:T | 0.0000035 | FALSE | TRUE | TRUE | TRUE | MISSENSE | FALSE |
| 5:96393159:C:T | 0.0000140 | FALSE | TRUE | FALSE | TRUE | MISSENSE | FALSE |
| 5:96393169:T:G | 0.0000035 | FALSE | TRUE | FALSE | TRUE | MISSENSE | FALSE |
| 5:96393172:A:C | 0.0000085 | TRUE | TRUE | TRUE | TRUE | STOP_GAINED | TRUE |
| 5:96393179:A:C | 0.0000105 | FALSE | TRUE | FALSE | TRUE | MISSENSE | FALSE |
| 5:96393186:G:A | 0.0000085 | FALSE | TRUE | FALSE | TRUE | MISSENSE | FALSE |
| 5:96393187:C:G | 0.0000035 | FALSE | TRUE | FALSE | TRUE | MISSENSE | FALSE |
| 5:96393210:G:A | 0.0000035 | FALSE | TRUE | FALSE | TRUE | MISSENSE | FALSE |
| 5:96393239:A:G | 0.0000140 | FALSE | TRUE | TRUE | TRUE | MISSENSE | FALSE |
| 5:96393257:C:A | 0.0000065 | FALSE | TRUE | FALSE | TRUE | MISSENSE | FALSE |
| 5:96393257:C:T | 0.0000140 | FALSE | TRUE | FALSE | TRUE | MISSENSE | FALSE |
| 5:96393258:G:A | 0.0000070 | TRUE | TRUE | TRUE | TRUE | STOP_GAINED | TRUE |
| 5:96393272:G:A | 0.0000035 | FALSE | TRUE | FALSE | TRUE | MISSENSE | FALSE |
| 5:96393284:T:C | 0.0000065 | FALSE | TRUE | FALSE | TRUE | MISSENSE | FALSE |
| 5:96393285:C:T | 0.0000130 | FALSE | TRUE | FALSE | TRUE | MISSENSE | FALSE |
| 5:96393300:T:A | 0.0000065 | FALSE | TRUE | FALSE | TRUE | MISSENSE | FALSE |
| 5:96393303:G:A | 0.0000140 | FALSE | TRUE | FALSE | TRUE | MISSENSE | FALSE |
| 5:96393308:C:A | 0.0000453 | FALSE | TRUE | FALSE | TRUE | MISSENSE | FALSE |
| 5:96393366:G:A | 0.0000194 | TRUE | TRUE | TRUE | TRUE | STOP_GAINED | TRUE |
| 5:96393378:C:G | 0.0000035 | FALSE | TRUE | FALSE | TRUE | MISSENSE | FALSE |
| 5:96394878:C:A | 0.0000070 | FALSE | TRUE | FALSE | TRUE | MISSENSE | FALSE |
| 5:96394882:C:G | 0.0000035 | FALSE | TRUE | FALSE | TRUE | MISSENSE | FALSE |
| 5:96394890:C:A | 0.0000035 | FALSE | TRUE | FALSE | TRUE | MISSENSE | FALSE |
| 5:96394893:C:T | 0.0000035 | FALSE | TRUE | FALSE | TRUE | MISSENSE | FALSE |
| 5:96394901:T:G | 0.0000035 | FALSE | TRUE | FALSE | TRUE | MISSENSE | FALSE |
| 5:96394911:C:T | 0.0000175 | FALSE | TRUE | FALSE | TRUE | MISSENSE | FALSE |
| 5:96394925:G:A | 0.0000175 | FALSE | TRUE | TRUE | TRUE | MISSENSE | FALSE |
| 5:96394934:C:T | 0.0000175 | FALSE | TRUE | TRUE | TRUE | MISSENSE | FALSE |
| 5:96394935:G:A | 0.0000035 | FALSE | TRUE | TRUE | TRUE | MISSENSE | FALSE |
| 5:96394941:G:A | 0.0000035 | TRUE | TRUE | TRUE | TRUE | STOP_GAINED | TRUE |
| 5:96394947:T:C | 0.0000065 | FALSE | TRUE | FALSE | TRUE | MISSENSE | FALSE |
| 5:96394964:G:T | 0.0000065 | FALSE | TRUE | FALSE | TRUE | MISSENSE | FALSE |
| 5:96394973:T:C | 0.0000035 | FALSE | TRUE | TRUE | TRUE | MISSENSE | FALSE |
| 5:96394988:C:T | 0.0000065 | TRUE | TRUE | TRUE | TRUE | STOP_GAINED | TRUE |
| 5:96394997:A:G | 0.0000035 | FALSE | TRUE | TRUE | TRUE | MISSENSE | FALSE |
| 5:96395003:C:T | 0.0000065 | FALSE | TRUE | TRUE | TRUE | MISSENSE | FALSE |
| 5:96395024:G:C | 0.0000065 | FALSE | TRUE | TRUE | TRUE | MISSENSE | FALSE |
| 5:96397344:T:C | 0.0000085 | FALSE | TRUE | FALSE | TRUE | MISSENSE | FALSE |
| 5:96397346:A:T | 0.0000105 | FALSE | TRUE | TRUE | TRUE | MISSENSE | FALSE |
| 5:96397347:T:A | 0.0000455 | FALSE | TRUE | FALSE | TRUE | MISSENSE | FALSE |
| 5:96397351:CAA:C | 0.0000194 | TRUE | TRUE | TRUE | TRUE | FRAMESHIFT | TRUE |
| 5:96397351:C:G | 0.0000254 | FALSE | TRUE | TRUE | TRUE | MISSENSE | FALSE |
| 5:96397357:C:G | 0.0000065 | FALSE | TRUE | TRUE | TRUE | MISSENSE | FALSE |
| 5:96397358:C:T | 0.0000085 | TRUE | TRUE | TRUE | TRUE | STOP_GAINED | TRUE |
| 5:96397362:T:C | 0.0000035 | FALSE | TRUE | FALSE | TRUE | MISSENSE | FALSE |
| 5:96397364:C:A | 0.0000065 | FALSE | TRUE | TRUE | TRUE | MISSENSE | FALSE |
| 5:96397372:G:T | 0.0000035 | FALSE | TRUE | FALSE | TRUE | MISSENSE | FALSE |
| 5:96397377:C:A | 0.0000065 | TRUE | TRUE | TRUE | TRUE | STOP_GAINED | TRUE |
| 5:96397380:C:T | 0.0000065 | FALSE | TRUE | TRUE | TRUE | MISSENSE | FALSE |
| 5:96397382:C:T | 0.0000035 | TRUE | TRUE | TRUE | TRUE | STOP_GAINED | TRUE |
| 5:96397390:AAC:A | 0.0000065 | TRUE | TRUE | TRUE | TRUE | FRAMESHIFT | TRUE |
| 5:96397392:C:T | 0.0000701 | FALSE | TRUE | FALSE | TRUE | MISSENSE | FALSE |
| 5:96397397:A:G | 0.0000065 | FALSE | TRUE | TRUE | TRUE | MISSENSE | FALSE |
| 5:96397424:G:C | 0.0000210 | FALSE | TRUE | FALSE | TRUE | MISSENSE | FALSE |
| 5:96397431:T:A | 0.0000035 | FALSE | TRUE | FALSE | TRUE | MISSENSE | FALSE |
| 5:96397432:A:T | 0.0000035 | FALSE | TRUE | TRUE | TRUE | MISSENSE | FALSE |
| 5:96397433:T:C | 0.0000070 | FALSE | TRUE | TRUE | TRUE | MISSENSE | FALSE |
| 5:96397436:C:G | 0.0000194 | FALSE | TRUE | FALSE | TRUE | MISSENSE | FALSE |
| 5:96397437:G:A | 0.0000315 | FALSE | TRUE | FALSE | TRUE | MISSENSE | FALSE |
| 5:96397439:T:C | 0.0000035 | FALSE | TRUE | FALSE | TRUE | MISSENSE | FALSE |
| 5:96397440:C:A | 0.0000065 | TRUE | TRUE | TRUE | TRUE | STOP_GAINED | TRUE |
| 5:96397451:A:G | 0.0000035 | FALSE | TRUE | TRUE | TRUE | MISSENSE | FALSE |
| 5:96397461:T:C | 0.0000035 | FALSE | TRUE | FALSE | TRUE | MISSENSE | FALSE |
| 5:96397463:C:T | 0.0000035 | FALSE | TRUE | FALSE | TRUE | MISSENSE | FALSE |
| 5:96398877:A:G | 0.0000065 | TRUE | TRUE | TRUE | TRUE | SPLICE_DONOR | TRUE |
| 5:96398887:GAAGT:G | 0.0000085 | TRUE | TRUE | TRUE | TRUE | FRAMESHIFT | TRUE |
| 5:96398897:T:C | 0.0000035 | FALSE | TRUE | FALSE | TRUE | MISSENSE | FALSE |
| 5:96398902:T:C | 0.0000035 | FALSE | TRUE | FALSE | TRUE | MISSENSE | FALSE |
| 5:96398911:C:A | 0.0000130 | FALSE | TRUE | TRUE | TRUE | MISSENSE | FALSE |
| 5:96398917:C:T | 0.0002172 | FALSE | TRUE | TRUE | TRUE | MISSENSE | FALSE |
| 5:96398920:G:A | 0.0000035 | FALSE | TRUE | FALSE | TRUE | MISSENSE | FALSE |
| 5:96398922:A:C | 0.0000035 | TRUE | TRUE | TRUE | TRUE | STOP_GAINED | TRUE |
| 5:96398929:A:G | 0.0000035 | FALSE | TRUE | TRUE | TRUE | MISSENSE | FALSE |
| 5:96398945:G:T | 0.0000085 | FALSE | TRUE | FALSE | TRUE | MISSENSE | FALSE |
| 5:96398966:T:G | 0.0000070 | FALSE | TRUE | FALSE | TRUE | MISSENSE | FALSE |
| 5:96398980:C:T | 0.0000035 | FALSE | TRUE | FALSE | TRUE | MISSENSE | FALSE |
| 5:96398983:T:A | 0.0000085 | FALSE | TRUE | FALSE | TRUE | MISSENSE | FALSE |
| 5:96399007:A:G | 0.0000035 | FALSE | TRUE | FALSE | TRUE | MISSENSE | FALSE |
| 5:96399008:T:C | 0.0000065 | FALSE | TRUE | FALSE | TRUE | MISSENSE | FALSE |
| 5:96399952:C:A | 0.0000065 | TRUE | TRUE | TRUE | TRUE | SPLICE_DONOR | TRUE |
| 5:96399958:C:G | 0.0000070 | FALSE | TRUE | FALSE | TRUE | MISSENSE | FALSE |
| 5:96399969:T:C | 0.0000035 | FALSE | TRUE | FALSE | TRUE | MISSENSE | FALSE |
| 5:96399978:C:T | 0.0000245 | FALSE | TRUE | FALSE | TRUE | MISSENSE | FALSE |
| 5:96399980:A:G | 0.0000453 | FALSE | TRUE | FALSE | TRUE | MISSENSE | FALSE |
| 5:96399993:T:A | 0.0000105 | TRUE | TRUE | TRUE | TRUE | STOP_GAINED | TRUE |
| 5:96399994:C:G | 0.0000070 | FALSE | TRUE | FALSE | TRUE | MISSENSE | FALSE |
| 5:96399996:C:T | 0.0000070 | FALSE | TRUE | FALSE | TRUE | MISSENSE | FALSE |
| 5:96399999:G:T | 0.0000175 | FALSE | TRUE | TRUE | TRUE | MISSENSE | FALSE |
| 5:96400002:C:G | 0.0000070 | FALSE | TRUE | FALSE | TRUE | MISSENSE | FALSE |
| 5:96400002:C:T | 0.0000596 | FALSE | TRUE | TRUE | TRUE | MISSENSE | FALSE |
| 5:96400010:C:G | 0.0000085 | FALSE | TRUE | TRUE | TRUE | MISSENSE | FALSE |
| 5:96400028:A:G | 0.0000065 | FALSE | TRUE | TRUE | TRUE | MISSENSE | FALSE |
| 5:96400032:C:T | 0.0000035 | FALSE | TRUE | FALSE | TRUE | MISSENSE | FALSE |
| 5:96400038:G:C | 0.0000070 | FALSE | TRUE | TRUE | TRUE | MISSENSE | FALSE |
| 5:96400047:C:A | 0.0000035 | FALSE | TRUE | TRUE | TRUE | MISSENSE | FALSE |
| 5:96400049:T:A | 0.0000035 | FALSE | TRUE | TRUE | TRUE | MISSENSE | FALSE |
| 5:96400052:A:G | 0.0000065 | FALSE | TRUE | TRUE | TRUE | MISSENSE | FALSE |
| 5:96400061:A:T | 0.0000035 | FALSE | TRUE | FALSE | TRUE | MISSENSE | FALSE |
| 5:96400070:C:T | 0.0000065 | FALSE | TRUE | FALSE | TRUE | MISSENSE | FALSE |
| 5:96400071:G:A | 0.0000070 | TRUE | TRUE | TRUE | TRUE | STOP_GAINED | TRUE |
| 5:96400091:G:T | 0.0000035 | FALSE | TRUE | TRUE | TRUE | MISSENSE | FALSE |
| 5:96400096:A:T | 0.0000259 | FALSE | TRUE | TRUE | TRUE | MISSENSE | FALSE |
| 5:96400100:T:C | 0.0000085 | FALSE | TRUE | FALSE | TRUE | MISSENSE | FALSE |
| 5:96400106:C:T | 0.0000130 | TRUE | TRUE | TRUE | TRUE | STOP_GAINED | TRUE |
| 5:96400128:G:A | 0.0000085 | FALSE | TRUE | FALSE | TRUE | MISSENSE | FALSE |
| 5:96400133:T:G | 0.0000175 | FALSE | TRUE | FALSE | TRUE | MISSENSE | FALSE |
| 5:96400142:G:C | 0.0000035 | FALSE | TRUE | FALSE | TRUE | MISSENSE | FALSE |
| 5:96400146:A:G | 0.0000070 | FALSE | TRUE | FALSE | TRUE | MISSENSE | FALSE |
| 5:96400156:G:C | 0.0000065 | FALSE | TRUE | TRUE | TRUE | MISSENSE | FALSE |
| 5:96400169:C:T | 0.0000035 | FALSE | TRUE | TRUE | TRUE | MISSENSE | FALSE |
| 5:96400170:G:A | 0.0000175 | TRUE | TRUE | TRUE | TRUE | STOP_GAINED | TRUE |
| 5:96408221:A:C | 0.0000035 | TRUE | TRUE | TRUE | TRUE | SPLICE_DONOR | TRUE |
| 5:96408226:G:T | 0.0000280 | FALSE | TRUE | TRUE | TRUE | MISSENSE | FALSE |
| 5:96408232:A:G | 0.0000130 | FALSE | TRUE | TRUE | TRUE | MISSENSE | FALSE |
| 5:96408238:A:G | 0.0000035 | FALSE | TRUE | TRUE | TRUE | MISSENSE | FALSE |
| 5:96408242:C:G | 0.0000065 | FALSE | TRUE | TRUE | TRUE | MISSENSE | FALSE |
| 5:96408242:C:T | 0.0000070 | FALSE | TRUE | TRUE | TRUE | MISSENSE | FALSE |
| 5:96408244:A:G | 0.0000280 | FALSE | TRUE | TRUE | TRUE | MISSENSE | FALSE |
| 5:96408251:C:T | 0.0000035 | FALSE | TRUE | TRUE | TRUE | MISSENSE | FALSE |
| 5:96408254:C:T | 0.0000035 | FALSE | TRUE | TRUE | TRUE | MISSENSE | FALSE |
| 5:96408256:G:T | 0.0000065 | FALSE | TRUE | TRUE | TRUE | MISSENSE | FALSE |
| 5:96408271:G:A | 0.0000035 | FALSE | TRUE | FALSE | TRUE | MISSENSE | FALSE |
| 5:96408272:C:T | 0.0000065 | FALSE | TRUE | TRUE | TRUE | MISSENSE | FALSE |
| 5:96408274:G:A | 0.0000105 | FALSE | TRUE | TRUE | TRUE | MISSENSE | FALSE |
| 5:96408283:G:C | 0.0000194 | FALSE | TRUE | TRUE | TRUE | MISSENSE | FALSE |
| 5:96408285:G:T | 0.0000085 | FALSE | TRUE | TRUE | TRUE | MISSENSE | FALSE |
| 5:96408288:C:CA | 0.0000065 | TRUE | TRUE | TRUE | TRUE | FRAMESHIFT | TRUE |
| 5:96408289:G:A | 0.0000666 | FALSE | TRUE | FALSE | TRUE | MISSENSE | FALSE |
| 5:96408292:T:A | 0.0000065 | FALSE | TRUE | FALSE | TRUE | MISSENSE | FALSE |
| 5:96408295:G:A | 0.0000035 | FALSE | TRUE | TRUE | TRUE | MISSENSE | FALSE |
| 5:96408296:T:A | 0.0000065 | FALSE | TRUE | TRUE | TRUE | MISSENSE | FALSE |
| 5:96408299:A:AG | 0.0000065 | TRUE | TRUE | TRUE | TRUE | FRAMESHIFT | TRUE |
| 5:96408303:A:T | 0.0000085 | FALSE | TRUE | FALSE | TRUE | MISSENSE | FALSE |
| 5:96408310:A:AG | 0.0000035 | TRUE | TRUE | TRUE | TRUE | FRAMESHIFT | TRUE |
| 5:96408312:G:C | 0.0000085 | FALSE | TRUE | TRUE | TRUE | MISSENSE | FALSE |
| 5:96408313:T:C | 0.0000065 | FALSE | TRUE | TRUE | TRUE | MISSENSE | FALSE |
| 5:96408316:G:A | 0.0000175 | FALSE | TRUE | FALSE | TRUE | MISSENSE | FALSE |
| 5:96408322:G:A | 0.0000070 | FALSE | TRUE | FALSE | TRUE | MISSENSE | FALSE |
| 5:96408323:T:C | 0.0001401 | FALSE | TRUE | FALSE | TRUE | MISSENSE | FALSE |
| 5:96408324:C:G | 0.0000035 | TRUE | TRUE | TRUE | TRUE | SPLICE_ACCEPTOR | TRUE |
| 5:96410771:T:TA | 0.0000105 | TRUE | TRUE | TRUE | TRUE | SPLICE_DONOR | TRUE |
| 5:96410773:C:T | 0.0000065 | TRUE | TRUE | TRUE | TRUE | SPLICE_DONOR | TRUE |
| 5:96410776:T:C | 0.0000035 | FALSE | TRUE | FALSE | TRUE | MISSENSE | FALSE |
| 5:96410785:C:T | 0.0000035 | FALSE | TRUE | TRUE | TRUE | MISSENSE | FALSE |
| 5:96410794:C:G | 0.0000070 | FALSE | TRUE | FALSE | TRUE | MISSENSE | FALSE |
| 5:96410797:C:A | 0.0000070 | TRUE | TRUE | TRUE | TRUE | STOP_GAINED | TRUE |
| 5:96410797:C:T | 0.0000035 | FALSE | TRUE | TRUE | TRUE | MISSENSE | FALSE |
| 5:96410808:G:C | 0.0000035 | FALSE | TRUE | TRUE | TRUE | MISSENSE | FALSE |
| 5:96410814:G:A | 0.0000035 | FALSE | TRUE | TRUE | TRUE | MISSENSE | FALSE |
| 5:96410818:G:T | 0.0000065 | FALSE | TRUE | TRUE | TRUE | MISSENSE | FALSE |
| 5:96410826:G:A | 0.0000035 | FALSE | TRUE | TRUE | TRUE | MISSENSE | FALSE |
| 5:96410839:C:A | 0.0000085 | FALSE | TRUE | FALSE | TRUE | MISSENSE | FALSE |
| 5:96410839:C:T | 0.0000105 | FALSE | TRUE | FALSE | TRUE | MISSENSE | FALSE |
| 5:96410840:G:C | 0.0000280 | TRUE | TRUE | TRUE | TRUE | STOP_GAINED | TRUE |
| 5:96410840:G:T | 0.0000065 | TRUE | TRUE | TRUE | TRUE | STOP_GAINED | TRUE |
| 5:96410842:A:G | 0.0000085 | FALSE | TRUE | TRUE | TRUE | MISSENSE | FALSE |
| 5:96410844:CAGGGGGAT:C | 0.0000070 | TRUE | TRUE | TRUE | TRUE | FRAMESHIFT | TRUE |
| 5:96410845:A:C | 0.0000210 | FALSE | TRUE | TRUE | TRUE | MISSENSE | FALSE |
| 5:96410856:C:A | 0.0000140 | FALSE | TRUE | TRUE | TRUE | MISSENSE | FALSE |
| 5:96410856:C:T | 0.0000070 | FALSE | TRUE | TRUE | TRUE | MISSENSE | FALSE |
| 5:96410857:C:A | 0.0000065 | FALSE | TRUE | TRUE | TRUE | MISSENSE | FALSE |
| 5:96410875:T:C | 0.0000035 | FALSE | TRUE | FALSE | TRUE | MISSENSE | FALSE |
| 5:96410878:T:A | 0.0000070 | FALSE | TRUE | TRUE | TRUE | MISSENSE | FALSE |
| 5:96410883:A:G | 0.0000105 | FALSE | TRUE | TRUE | TRUE | MISSENSE | FALSE |
| 5:96410887:T:C | 0.0000065 | FALSE | TRUE | TRUE | TRUE | MISSENSE | FALSE |
| 5:96410895:C:A | 0.0000065 | FALSE | TRUE | TRUE | TRUE | MISSENSE | FALSE |
| 5:96410901:G:A | 0.0000035 | FALSE | TRUE | TRUE | TRUE | MISSENSE | FALSE |
| 5:96410907:C:A | 0.0000035 | FALSE | TRUE | TRUE | TRUE | MISSENSE | FALSE |
| 5:96410907:C:T | 0.0000035 | FALSE | TRUE | TRUE | TRUE | MISSENSE | FALSE |
| 5:96410910:T:TC | 0.0000130 | TRUE | TRUE | TRUE | TRUE | FRAMESHIFT | TRUE |
| 5:96410913:C:T | 0.0000065 | FALSE | TRUE | TRUE | TRUE | MISSENSE | FALSE |
| 5:96410929:C:T | 0.0000453 | FALSE | TRUE | FALSE | TRUE | MISSENSE | FALSE |
| 5:96410934:C:T | 0.0000035 | FALSE | TRUE | FALSE | TRUE | MISSENSE | FALSE |
| 5:96410935:G:A | 0.0000035 | FALSE | TRUE | TRUE | TRUE | MISSENSE | FALSE |
| 5:96410935:G:GC | 0.0000035 | TRUE | TRUE | TRUE | TRUE | FRAMESHIFT | TRUE |
| 5:96410940:C:A | 0.0000035 | FALSE | TRUE | TRUE | TRUE | MISSENSE | FALSE |
| 5:96410941:C:T | 0.0000085 | FALSE | TRUE | TRUE | TRUE | MISSENSE | FALSE |
| 5:96410946:C:T | 0.0000105 | FALSE | TRUE | TRUE | TRUE | MISSENSE | FALSE |
| 5:96410949:G:A | 0.0000175 | FALSE | TRUE | FALSE | TRUE | MISSENSE | FALSE |
| 5:96410959:C:T | 0.0000525 | FALSE | TRUE | FALSE | TRUE | MISSENSE | FALSE |
| 5:96410965:T:G | 0.0000070 | FALSE | TRUE | FALSE | TRUE | MISSENSE | FALSE |
| 5:96410970:C:T | 0.0000035 | FALSE | TRUE | TRUE | TRUE | MISSENSE | FALSE |
| 5:96410974:TC:T | 0.0000712 | TRUE | TRUE | TRUE | TRUE | FRAMESHIFT | TRUE |
| 5:96410976:C:G | 0.0000065 | FALSE | TRUE | TRUE | TRUE | MISSENSE | FALSE |
| 5:96410983:T:TC | 0.0000035 | TRUE | TRUE | TRUE | TRUE | FRAMESHIFT | TRUE |
| 5:96410985:C:T | 0.0000065 | FALSE | TRUE | TRUE | TRUE | MISSENSE | FALSE |
| 5:96412322:T:C | 0.0000130 | FALSE | TRUE | FALSE | TRUE | MISSENSE | FALSE |
| 5:96412328:C:T | 0.0000035 | FALSE | TRUE | TRUE | TRUE | MISSENSE | FALSE |
| 5:96412332:A:G | 0.0000130 | FALSE | TRUE | FALSE | TRUE | MISSENSE | FALSE |
| 5:96412355:C:T | 0.0000065 | FALSE | TRUE | FALSE | TRUE | MISSENSE | FALSE |
| 5:96412356:G:A | 0.0000175 | FALSE | TRUE | TRUE | TRUE | MISSENSE | FALSE |
| 5:96412362:G:A | 0.0000130 | FALSE | TRUE | TRUE | TRUE | MISSENSE | FALSE |
| 5:96412362:G:T | 0.0000035 | FALSE | TRUE | TRUE | TRUE | MISSENSE | FALSE |
| 5:96412364:C:G | 0.0000169 | FALSE | TRUE | TRUE | TRUE | MISSENSE | FALSE |
| 5:96412379:C:G | 0.0000070 | FALSE | TRUE | TRUE | TRUE | MISSENSE | FALSE |
| 5:96412382:T:C | 0.0000065 | FALSE | TRUE | TRUE | TRUE | MISSENSE | FALSE |
| 5:96412385:T:C | 0.0000169 | FALSE | TRUE | TRUE | TRUE | MISSENSE | FALSE |
| 5:96412395:C:T | 0.0000065 | FALSE | TRUE | TRUE | TRUE | MISSENSE | FALSE |
| 5:96412400:C:T | 0.0000085 | FALSE | TRUE | TRUE | TRUE | MISSENSE | FALSE |
| 5:96412416:C:T | 0.0000130 | FALSE | TRUE | TRUE | TRUE | MISSENSE | FALSE |
| 5:96412418:A:G | 0.0000140 | FALSE | TRUE | TRUE | TRUE | MISSENSE | FALSE |
| 5:96412419:C:T | 0.0000070 | FALSE | TRUE | TRUE | TRUE | MISSENSE | FALSE |
| 5:96412420:G:T | 0.0000105 | FALSE | TRUE | TRUE | TRUE | MISSENSE | FALSE |
| 5:96412425:C:T | 0.0000085 | FALSE | TRUE | FALSE | TRUE | MISSENSE | FALSE |
| 5:96412436:C:G | 0.0000065 | FALSE | TRUE | TRUE | TRUE | MISSENSE | FALSE |
| 5:96412436:C:T | 0.0000070 | FALSE | TRUE | TRUE | TRUE | MISSENSE | FALSE |
| 5:96412437:C:CA | 0.0000065 | TRUE | TRUE | TRUE | TRUE | FRAMESHIFT | TRUE |
| 5:96412437:C:T | 0.0000035 | FALSE | TRUE | TRUE | TRUE | MISSENSE | FALSE |
| 5:96412439:A:G | 0.0000085 | FALSE | TRUE | TRUE | TRUE | MISSENSE | FALSE |
| 5:96412440:T:C | 0.0000065 | FALSE | TRUE | FALSE | TRUE | MISSENSE | FALSE |
| 5:96412443:A:T | 0.0000065 | FALSE | TRUE | TRUE | TRUE | MISSENSE | FALSE |
| 5:96412445:C:T | 0.0000035 | FALSE | TRUE | FALSE | TRUE | MISSENSE | FALSE |
| 5:96412448:G:A | 0.0000070 | FALSE | TRUE | TRUE | TRUE | MISSENSE | FALSE |
| 5:96412452:C:A | 0.0000105 | TRUE | TRUE | TRUE | TRUE | STOP_GAINED | TRUE |
| 5:96412454:A:AT | 0.0000065 | TRUE | TRUE | TRUE | TRUE | FRAMESHIFT | TRUE |
| 5:96412454:A:G | 0.0000035 | FALSE | TRUE | TRUE | TRUE | MISSENSE | FALSE |
| 5:96412455:T:C | 0.0000712 | FALSE | TRUE | FALSE | TRUE | MISSENSE | FALSE |
| 5:96412463:G:A | 0.0000140 | FALSE | TRUE | TRUE | TRUE | MISSENSE | FALSE |
| 5:96412464:T:A | 0.0000035 | FALSE | TRUE | TRUE | TRUE | MISSENSE | FALSE |
| 5:96412480:C:G | 0.0000035 | FALSE | TRUE | TRUE | TRUE | MISSENSE | FALSE |
| 5:96412484:C:A | 0.0000085 | FALSE | TRUE | TRUE | TRUE | MISSENSE | FALSE |
| 5:96412488:T:C | 0.0000035 | FALSE | TRUE | FALSE | TRUE | MISSENSE | FALSE |
| 5:96416041:T:A | 0.0001296 | FALSE | TRUE | TRUE | TRUE | MISSENSE | FALSE |
| 5:96416041:T:G | 0.0000065 | FALSE | TRUE | TRUE | TRUE | MISSENSE | FALSE |
| 5:96416047:T:C | 0.0000070 | FALSE | TRUE | FALSE | TRUE | MISSENSE | FALSE |
| 5:96416050:T:C | 0.0000130 | FALSE | TRUE | TRUE | TRUE | MISSENSE | FALSE |
| 5:96416051:A:G | 0.0000070 | FALSE | TRUE | TRUE | TRUE | MISSENSE | FALSE |
| 5:96416053:G:A | 0.0000194 | FALSE | TRUE | TRUE | TRUE | MISSENSE | FALSE |
| 5:96416054:C:A | 0.0000035 | FALSE | TRUE | TRUE | TRUE | MISSENSE | FALSE |
| 5:96416054:C:G | 0.0000065 | FALSE | TRUE | TRUE | TRUE | MISSENSE | FALSE |
| 5:96416057:C:T | 0.0000194 | FALSE | TRUE | FALSE | TRUE | MISSENSE | FALSE |
| 5:96416059:C:A | 0.0000105 | FALSE | TRUE | TRUE | TRUE | MISSENSE | FALSE |
| 5:96416062:A:G | 0.0000070 | FALSE | TRUE | FALSE | TRUE | MISSENSE | FALSE |
| 5:96416063:C:T | 0.0000070 | FALSE | TRUE | TRUE | TRUE | MISSENSE | FALSE |
| 5:96416066:C:G | 0.0000035 | FALSE | TRUE | TRUE | TRUE | MISSENSE | FALSE |
| 5:96416066:C:T | 0.0000210 | FALSE | TRUE | TRUE | TRUE | MISSENSE | FALSE |
| 5:96416067:G:T | 0.0000035 | TRUE | TRUE | TRUE | TRUE | STOP_GAINED | TRUE |
| 5:96416068:C:T | 0.0000035 | FALSE | TRUE | TRUE | TRUE | MISSENSE | FALSE |
| 5:96416069:A:G | 0.0000035 | FALSE | TRUE | TRUE | TRUE | MISSENSE | FALSE |
| 5:96416072:T:A | 0.0000035 | TRUE | TRUE | TRUE | TRUE | STOP_GAINED | TRUE |
| 5:96416075:G:A | 0.0000035 | FALSE | TRUE | FALSE | TRUE | MISSENSE | FALSE |
| 5:96416092:G:A | 0.0001787 | FALSE | TRUE | TRUE | TRUE | MISSENSE | FALSE |
| 5:96416105:C:A | 0.0000035 | FALSE | TRUE | TRUE | TRUE | MISSENSE | FALSE |
| 5:96416105:C:T | 0.0000035 | FALSE | TRUE | TRUE | TRUE | MISSENSE | FALSE |
| 5:96416117:C:T | 0.0000035 | FALSE | TRUE | TRUE | TRUE | MISSENSE | FALSE |
| 5:96421879:CT:C | 0.0000035 | TRUE | TRUE | TRUE | TRUE | FRAMESHIFT | TRUE |
| 5:96421881:T:A | 0.0000035 | TRUE | TRUE | TRUE | TRUE | STOP_GAINED | TRUE |
| 5:96421882:G:T | 0.0000105 | FALSE | TRUE | TRUE | TRUE | MISSENSE | FALSE |
| 5:96421887:C:T | 0.0000105 | FALSE | TRUE | FALSE | TRUE | MISSENSE | FALSE |
| 5:96421893:TG:T | 0.0000065 | TRUE | TRUE | TRUE | TRUE | FRAMESHIFT | TRUE |
| 5:96421896:G:A | 0.0000070 | FALSE | TRUE | FALSE | TRUE | MISSENSE | FALSE |
| 5:96421898:T:A | 0.0000070 | FALSE | TRUE | TRUE | TRUE | MISSENSE | FALSE |
| 5:96421902:A:T | 0.0000035 | FALSE | TRUE | TRUE | TRUE | MISSENSE | FALSE |
| 5:96421904:C:T | 0.0000105 | FALSE | TRUE | TRUE | TRUE | MISSENSE | FALSE |
| 5:96421905:G:A | 0.0000194 | TRUE | TRUE | TRUE | TRUE | STOP_GAINED | TRUE |
| 5:96421908:G:A | 0.0000065 | FALSE | TRUE | TRUE | TRUE | MISSENSE | FALSE |
| 5:96421914:G:A | 0.0000194 | FALSE | TRUE | TRUE | TRUE | MISSENSE | FALSE |
| 5:96421916:T:C | 0.0000070 | FALSE | TRUE | TRUE | TRUE | MISSENSE | FALSE |
| 5:96421916:T:G | 0.0000105 | FALSE | TRUE | TRUE | TRUE | MISSENSE | FALSE |
| 5:96421922:T:C | 0.0000035 | FALSE | TRUE | TRUE | TRUE | MISSENSE | FALSE |
| 5:96421932:T:A | 0.0000065 | FALSE | TRUE | TRUE | TRUE | MISSENSE | FALSE |
| 5:96421934:A:C | 0.0000169 | FALSE | TRUE | FALSE | TRUE | MISSENSE | FALSE |
| 5:96421940:TA:T | 0.0000065 | TRUE | TRUE | TRUE | TRUE | FRAMESHIFT | TRUE |
| 5:96421941:A:G | 0.0000350 | FALSE | TRUE | TRUE | TRUE | MISSENSE | FALSE |
| 5:96421946:G:C | 0.0000210 | FALSE | TRUE | TRUE | TRUE | MISSENSE | FALSE |
| 5:96421950:C:G | 0.0000035 | FALSE | TRUE | FALSE | TRUE | MISSENSE | FALSE |
| 5:96421951:TG:T | 0.0000065 | TRUE | TRUE | TRUE | TRUE | FRAMESHIFT | TRUE |
| 5:96421954:AT:A | 0.0000065 | TRUE | TRUE | TRUE | TRUE | FRAMESHIFT | TRUE |
| 5:96423315:A:G | 0.0003818 | FALSE | TRUE | TRUE | TRUE | MISSENSE | FALSE |
| 5:96423317:T:C | 0.0000806 | FALSE | TRUE | TRUE | TRUE | MISSENSE | FALSE |
| 5:96423320:G:A | 0.0000035 | FALSE | TRUE | FALSE | TRUE | MISSENSE | FALSE |
| 5:96423326:A:T | 0.0000169 | FALSE | TRUE | TRUE | TRUE | MISSENSE | FALSE |
| 5:96423332:G:A | 0.0000455 | FALSE | TRUE | TRUE | TRUE | MISSENSE | FALSE |
| 5:96423336:G:A | 0.0000105 | FALSE | TRUE | TRUE | TRUE | MISSENSE | FALSE |
| 5:96423337:A:T | 0.0000085 | FALSE | TRUE | TRUE | TRUE | MISSENSE | FALSE |
| 5:96423338:T:A | 0.0000085 | FALSE | TRUE | TRUE | TRUE | MISSENSE | FALSE |
| 5:96423354:CA:C | 0.0000065 | TRUE | TRUE | TRUE | TRUE | FRAMESHIFT | TRUE |
| 5:96423356:T:A | 0.0000085 | FALSE | TRUE | TRUE | TRUE | MISSENSE | FALSE |
| 5:96423363:C:T | 0.0000175 | FALSE | TRUE | FALSE | TRUE | MISSENSE | FALSE |
| 5:96423375:C:T | 0.0000035 | FALSE | TRUE | FALSE | TRUE | MISSENSE | FALSE |
| 5:96423377:C:T | 0.0000085 | FALSE | TRUE | TRUE | TRUE | MISSENSE | FALSE |
| 5:96423386:G:A | 0.0000070 | FALSE | TRUE | TRUE | TRUE | MISSENSE | FALSE |
| 5:96423387:T:A | 0.0000035 | FALSE | TRUE | FALSE | TRUE | MISSENSE | FALSE |
| 5:96423392:C:T | 0.0000065 | FALSE | TRUE | TRUE | TRUE | MISSENSE | FALSE |
| 5:96423393:C:T | 0.0000385 | FALSE | TRUE | TRUE | TRUE | MISSENSE | FALSE |
| 5:96423401:CAA:C | 0.0000035 | TRUE | TRUE | TRUE | TRUE | FRAMESHIFT | TRUE |
| 5:96423409:T:C | 0.0000065 | FALSE | TRUE | FALSE | TRUE | MISSENSE | FALSE |
| 5:96423410:A:G | 0.0000070 | FALSE | TRUE | FALSE | TRUE | MISSENSE | FALSE |
| 5:96423411:T:A | 0.0000105 | FALSE | TRUE | FALSE | TRUE | MISSENSE | FALSE |
| 5:96423416:T:G | 0.0000085 | FALSE | TRUE | TRUE | TRUE | MISSENSE | FALSE |
| 5:96423431:G:A | 0.0000065 | FALSE | TRUE | TRUE | TRUE | MISSENSE | FALSE |
| 5:96423443:G:A | 0.0000245 | FALSE | TRUE | FALSE | TRUE | MISSENSE | FALSE |
| 5:96423452:G:T | 0.0000245 | FALSE | TRUE | TRUE | TRUE | MISSENSE | FALSE |
| 5:96425819:C:G | 0.0000035 | TRUE | TRUE | TRUE | TRUE | SPLICE_DONOR | TRUE |
| 5:96425827:C:G | 0.0000035 | FALSE | TRUE | TRUE | TRUE | MISSENSE | FALSE |
| 5:96425827:C:T | 0.0000105 | TRUE | TRUE | TRUE | TRUE | STOP_GAINED | TRUE |
| 5:96425836:T:A | 0.0000070 | FALSE | TRUE | FALSE | TRUE | MISSENSE | FALSE |
| 5:96425836:T:C | 0.0000085 | FALSE | TRUE | FALSE | TRUE | MISSENSE | FALSE |
| 5:96425837:T:G | 0.0000175 | FALSE | TRUE | TRUE | TRUE | MISSENSE | FALSE |
| 5:96425841:C:T | 0.0002347 | FALSE | TRUE | FALSE | TRUE | MISSENSE | FALSE |
| 5:96425842:A:T | 0.0000175 | FALSE | TRUE | FALSE | TRUE | MISSENSE | FALSE |
| 5:96425843:TG:T | 0.0000130 | TRUE | TRUE | TRUE | TRUE | FRAMESHIFT | TRUE |
| 5:96425845:G:A | 0.0000065 | FALSE | TRUE | TRUE | TRUE | MISSENSE | FALSE |
| 5:96425851:T:C | 0.0000194 | FALSE | TRUE | FALSE | TRUE | MISSENSE | FALSE |
| 5:96425855:A:C | 0.0000175 | FALSE | TRUE | TRUE | TRUE | MISSENSE | FALSE |
| 5:96425887:C:G | 0.0000065 | FALSE | TRUE | TRUE | TRUE | MISSENSE | FALSE |
| 5:96425887:C:T | 0.0000140 | FALSE | TRUE | TRUE | TRUE | MISSENSE | FALSE |
| 5:96425888:G:A | 0.0000140 | FALSE | TRUE | TRUE | TRUE | MISSENSE | FALSE |
| 5:96425891:T:A | 0.0000035 | TRUE | TRUE | TRUE | TRUE | STOP_GAINED | TRUE |
| 5:96425892:AC:A | 0.0000070 | TRUE | TRUE | TRUE | TRUE | FRAMESHIFT | TRUE |
| 5:96425899:T:A | 0.0000035 | FALSE | TRUE | FALSE | TRUE | MISSENSE | FALSE |
| 5:96425920:G:A | 0.0000259 | FALSE | TRUE | FALSE | TRUE | MISSENSE | FALSE |
| 5:96425929:A:G | 0.0000065 | FALSE | TRUE | TRUE | TRUE | MISSENSE | FALSE |
| 5:96429214:C:T | 0.0000035 | FALSE | TRUE | TRUE | TRUE | MISSENSE | FALSE |
| 5:96429215:G:A | 0.0000583 | FALSE | TRUE | TRUE | TRUE | MISSENSE | FALSE |
| 5:96429215:G:C | 0.0000035 | FALSE | TRUE | FALSE | TRUE | MISSENSE | FALSE |
| 5:96429217:T:C | 0.0000105 | FALSE | TRUE | FALSE | TRUE | MISSENSE | FALSE |
| 5:96429220:T:C | 0.0000070 | FALSE | TRUE | FALSE | TRUE | MISSENSE | FALSE |
| 5:96429228:T:A | 0.0000035 | FALSE | TRUE | TRUE | TRUE | MISSENSE | FALSE |
| 5:96429238:G:A | 0.0000085 | FALSE | TRUE | TRUE | TRUE | MISSENSE | FALSE |
| 5:96429242:T:C | 0.0000085 | FALSE | TRUE | FALSE | TRUE | MISSENSE | FALSE |
| 5:96429243:A:T | 0.0000035 | FALSE | TRUE | FALSE | TRUE | MISSENSE | FALSE |
| 5:96429244:T:C | 0.0000070 | FALSE | TRUE | FALSE | TRUE | MISSENSE | FALSE |
| 5:96429252:A:C | 0.0000035 | FALSE | TRUE | FALSE | TRUE | MISSENSE | FALSE |
| 5:96429253:C:G | 0.0000035 | FALSE | TRUE | FALSE | TRUE | MISSENSE | FALSE |
| 5:96429255:C:A | 0.0000035 | FALSE | TRUE | TRUE | TRUE | MISSENSE | FALSE |
| 5:96429256:CT:C | 0.0000065 | TRUE | TRUE | TRUE | TRUE | FRAMESHIFT | TRUE |
| 5:96429259:C:T | 0.0000065 | FALSE | TRUE | TRUE | TRUE | MISSENSE | FALSE |
| 5:96429260:G:A | 0.0000065 | TRUE | TRUE | TRUE | TRUE | STOP_GAINED | TRUE |
| 5:96429262:G:C | 0.0000245 | FALSE | TRUE | TRUE | TRUE | MISSENSE | FALSE |
| 5:96429264:C:A | 0.0000130 | FALSE | TRUE | FALSE | TRUE | MISSENSE | FALSE |
| 5:96429265:C:T | 0.0000085 | FALSE | TRUE | FALSE | TRUE | MISSENSE | FALSE |
| 5:96429269:TG:T | 0.0000130 | TRUE | TRUE | TRUE | TRUE | FRAMESHIFT | TRUE |
| 5:96429272:G:A | 0.0000035 | FALSE | TRUE | FALSE | TRUE | MISSENSE | FALSE |
| 5:96429283:T:C | 0.0000035 | FALSE | TRUE | FALSE | TRUE | MISSENSE | FALSE |
| 5:96429287:T:G | 0.0000035 | FALSE | TRUE | FALSE | TRUE | MISSENSE | FALSE |
| 5:96429290:A:G | 0.0000035 | FALSE | TRUE | FALSE | TRUE | MISSENSE | FALSE |
| 5:96429299:G:T | 0.0000070 | FALSE | TRUE | TRUE | TRUE | MISSENSE | FALSE |
| 5:96429310:G:A | 0.0000389 | FALSE | TRUE | FALSE | TRUE | MISSENSE | FALSE |
| 5:96429310:G:T | 0.0000070 | TRUE | TRUE | TRUE | TRUE | STOP_GAINED | TRUE |
| 5:96429319:T:C | 0.0000070 | TRUE | TRUE | TRUE | TRUE | SPLICE_ACCEPTOR | TRUE |
| 5:96432082:C:T | 0.0000035 | TRUE | TRUE | TRUE | TRUE | SPLICE_DONOR | TRUE |
| 5:96432105:A:T | 0.0000140 | FALSE | TRUE | FALSE | TRUE | MISSENSE | FALSE |
| 5:96432108:G:A | 0.0000035 | FALSE | TRUE | FALSE | TRUE | MISSENSE | FALSE |
| 5:96432113:C:G | 0.0000194 | FALSE | TRUE | FALSE | TRUE | MISSENSE | FALSE |
| 5:96432115:T:C | 0.0000065 | FALSE | TRUE | FALSE | TRUE | MISSENSE | FALSE |
| 5:96432118:C:G | 0.0000065 | FALSE | TRUE | FALSE | TRUE | MISSENSE | FALSE |
| 5:96432861:A:C | 0.0000065 | TRUE | TRUE | TRUE | TRUE | SPLICE_DONOR | TRUE |
| 5:96432869:CA:C | 0.0000085 | TRUE | TRUE | TRUE | TRUE | FRAMESHIFT | TRUE |
| 5:96432870:A:G | 0.0000070 | FALSE | TRUE | FALSE | TRUE | MISSENSE | FALSE |
| 5:96432874:G:A | 0.0000070 | FALSE | TRUE | FALSE | TRUE | MISSENSE | FALSE |
| 5:96432876:T:C | 0.0000130 | FALSE | TRUE | FALSE | TRUE | MISSENSE | FALSE |
| 5:96432877:C:T | 0.0000035 | FALSE | TRUE | FALSE | TRUE | MISSENSE | FALSE |
| 5:96432886:G:C | 0.0000070 | FALSE | TRUE | FALSE | TRUE | MISSENSE | FALSE |
| 5:96432888:T:G | 0.0001166 | FALSE | TRUE | FALSE | TRUE | MISSENSE | FALSE |
| 5:96432889:C:T | 0.0000085 | FALSE | TRUE | FALSE | TRUE | MISSENSE | FALSE |
| 5:96432894:G:T | 0.0000085 | FALSE | TRUE | FALSE | TRUE | MISSENSE | FALSE |
| 5:96432895:C:A | 0.0000105 | FALSE | TRUE | FALSE | TRUE | MISSENSE | FALSE |
| 5:96432896:G:C | 0.0001878 | FALSE | TRUE | FALSE | TRUE | MISSENSE | FALSE |
| 5:96432897:A:T | 0.0000130 | FALSE | TRUE | FALSE | TRUE | MISSENSE | FALSE |
| 5:96432909:G:T | 0.0000105 | FALSE | TRUE | FALSE | TRUE | MISSENSE | FALSE |
| 5:96432913:C:A | 0.0000035 | TRUE | TRUE | TRUE | TRUE | STOP_GAINED | TRUE |
| 5:96432918:C:T | 0.0000490 | FALSE | TRUE | TRUE | TRUE | MISSENSE | FALSE |
| 5:96432921:C:G | 0.0000065 | FALSE | TRUE | FALSE | TRUE | MISSENSE | FALSE |
| 5:96432922:C:T | 0.0000140 | FALSE | TRUE | TRUE | TRUE | MISSENSE | FALSE |
| 5:96432927:A:G | 0.0000035 | FALSE | TRUE | FALSE | TRUE | MISSENSE | FALSE |
| 5:96432948:A:G | 0.0000085 | FALSE | TRUE | FALSE | TRUE | MISSENSE | FALSE |
| 5:96432952:A:G | 0.0000035 | FALSE | TRUE | FALSE | TRUE | MISSENSE | FALSE |
| 5:96432957:CT:C | 0.0000065 | TRUE | TRUE | TRUE | TRUE | FRAMESHIFT | TRUE |
| 5:96432971:AC:A | 0.0000259 | TRUE | TRUE | TRUE | TRUE | FRAMESHIFT | TRUE |
| 5:96432981:G:GCA | 0.0000035 | TRUE | TRUE | TRUE | TRUE | FRAMESHIFT | TRUE |
| 5:96432981:G:T | 0.0000140 | FALSE | TRUE | FALSE | TRUE | MISSENSE | FALSE |
| 5:96432985:A:G | 0.0000070 | FALSE | TRUE | FALSE | TRUE | MISSENSE | FALSE |
| 5:96432993:CAA:C | 0.0000065 | TRUE | TRUE | TRUE | TRUE | FRAMESHIFT | TRUE |
| 5:96433008:G:A | 0.0000065 | FALSE | TRUE | FALSE | TRUE | MISSENSE | FALSE |
| 5:96433040:C:T | 0.0000065 | TRUE | TRUE | TRUE | TRUE | START_LOST | TRUE |
| 5:96433041:A:G | 0.0000035 | TRUE | TRUE | TRUE | TRUE | START_LOST | TRUE |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having obesity and/or elevated body mass index (BMI), wherein the subject is heterozygous for a proprotein convertase subtilisin/kexin type 1 (PCSK1) variant genomic nucleic acid molecule associated with an increased risk of developing obesity and/or elevated BMI, comprising administering an agonist of the leptin-melanocortin signaling pathway to the subject.

2. (canceled)

3. The method according to claim 1, wherein the agonist of the leptin-melanocortin signaling pathway is a melanocortin-4 receptor (MC4R) agonist or a proprotein convertase subtilisin/kexin type 1 (PCSK1) agonist.

4. The method according to claim 3, wherein the MC4R agonist comprises a protein, a peptide, a nucleic acid molecule, or a small molecule.

5. The method according to claim 4, wherein the protein is a peptide analog of MC4R.

6. The method according to claim 4, wherein the MC4R agonist is setmelanotide, a peptide comprising the amino acid sequence His-Phe-Arg-Trp, 1,2,3R,4-tetrahydroisoquinoline-3-carboxylic acid, or ALB-127158(a).

7-14. (canceled)

15. The method according to claim 1, wherein the PSCK1 loss-of-function variant genomic nucleic acid molecule is a protein truncating variant genomic nucleic acid molecule.

16-30. (canceled)

31. A method of identifying susceptibility of a subject having obesity and/or elevated body mass index (BMI) to treatment with an agonist of the leptin-melanocortin signaling pathway, the method comprising detecting the presence or absence of one copy of a PCSK1 variant nucleic acid molecule associated with an increased risk of developing obesity and/or elevated BMI in a biological sample obtained from the subject, wherein when one copy of a PCSK1 variant nucleic acid molecule is detected, the subject is susceptible to treatment with the agonist of the leptin-melanocortin signaling pathway.

32. (canceled)

33. The method according to claim 32, wherein the PCSK1 variant nucleic acid molecule associated with an increased risk of developing obesity and/or elevated BMI is a PCSK1 loss-of-function variant nucleic acid molecule or a PCSK1 missense variant nucleic acid molecule.

34. The method according to, claim 31, wherein detecting the PCSK1 variant nucleic acid molecule associated with an increased risk of developing obesity and/or elevated BMI comprises detecting a PCSK1 variant genomic nucleic acid molecule associated with an increased risk of developing obesity and/or elevated BMI, a PCSK1 variant mRNA molecule associated with an increased risk of developing obesity and/or elevated BMI, a PCSK1 variant cDNA molecule produced from the mRNA molecule, and/or a PCSK1 variant polypeptide associated with an increased risk of developing obesity and/or elevated BMI, by:
obtaining or having obtained a biological sample from the subject; and
performing or having performed an assay on the biological sample to determine whether the subject is heterozygous for a PCSK1 variant nucleic acid molecule or PCSK1 variant polypeptide associated with an increased risk of developing obesity and/or elevated BMI.

35. The method according to claim 34, wherein the assay is a sequence analysis or genotyping assay for nucleic acid molecules.

36. The method according to claim 34, wherein the assay is an immunoassay for polypeptides.

37-43. (canceled)

44. The method according to, claim 31, further comprising administering the agonist of the leptin-melanocortin signaling pathway to the subject identified as susceptible to the leptin-melanocortin signaling pathway agonist treatment.

45. The method according to, claim 31, wherein the agonist of the leptin-melanocortin signaling pathway is a melanocortin-4 receptor (MC4R) agonist or a PCSK1 agonist.

46. The method according to claim 45, wherein the MC4R agonist comprises a protein, a peptide, a nucleic acid molecule, or a small molecule.

47. The method according to claim 46, wherein the protein is a peptide analog of MC4R.

48. The method according to claim 46, wherein the peptide MC4R agonist is setmelanotide, a peptide comprising the amino acid sequence His-Phe-Arg-Trp, 1,2,3R,4-tetrahydroisoquinoline-3-carboxylic acid, or ALB-127158(a).

49-51. (canceled)

52. The method according to claim 1, wherein the PCSK1 variant genomic nucleic acid molecule does not comprise 5:96421905:G:A according to the GRCh38/hg38 human genome assembly.

53. The method according to claim 1, wherein the PCSK1 variant genomic nucleic acid molecule comprises 5:96393172:A:C, 5:96393258:G:A, 5:96393366:G:A, 5:96394941:G:A, 5:96394988:C:T, 5:96397351:CAA:C, 5:96397358:C:T, 5:96397377:C:A, 5:96397382:C:T, 5:96397390:AAC:A, 5:96397440:C:A, 5:96398887:GAAGT:G, 5:96398922:A:C, 5:96399952:C:A, 5:96399993:T:A, 5:96400071:G:A, 5:96400106:C:T, 5:96408221:A:C, 5:96408288:C:CA, 5:96408299:A:AG, 5:96408310:A:AG, 5:96408324:C:G, 5:96410771:T:TA, 5:96410797:C:A, 5:96410840:G:T, 5:96410844:CAGGGGGAT:C, 5:96410910:T:TC, 5:96410935:G:GC, 5:96410974:TC:T, 5:96410983:T:TC, 5:96412437:C:CA, 5:96412454:A:AT, 5:96416067:G:T, 5:96416072:T:A, 5:96421879:CT:C, 5:96421881:T:A, 5:96421893:TG:T, 5:96421940:TA:T, 5:96421951:TG:T, 5:96421954:AT:A, 5:96423354:CA:C, 5:96423401:CAA:C, 5:96425819:C:G, 5:96425827:C:T, 5:96425843:TG:T, 5:96425891:T:A, 5:96425892:AC:A, 5:96429256:CT:C, 5:96429260:G:A, 5:96429269:TG:T, 5:96429310:G:T, 5:96429319:T:C, 5:96432082:C:T, 5:96432861:A:C, 5:96432869:CA:C, 5:96432913:C:A, 5:96432957:CT:C, 5:96432971:AC:A, 5:96432981:G:GCA, 5:96432993:CAA:C, and/or 5:96433040:C:T according to the GRCh38/hg38 human genome assembly.

54. A method of treating a subject having obesity and/or elevated body mass index (BMI), wherein the subject is heterozygous for a proprotein convertase subtilisin/kexin type 1 (PCSK1) variant genomic nucleic acid molecule associated with an increased risk of developing obesity and/or elevated BMI, the method comprising:
detecting the presence of a PCSK1 variant genomic nucleic acid molecule associated with an increased risk of developing obesity and/or elevated BMI in a biological sample from the subject; and
administering an MC4R agonist to the subject that is heterozygous for the PCSK1 variant genomic nucleic acid molecule associated with an increased risk of developing obesity and/or elevated BMI,
wherein the PCSK1 variant genomic nucleic acid molecule comprises 5:96393172:A:C, 5:96393258:G:A, 5:96393366:G:A, 5:96394941:G:A, 5:96394988:C:T, 5:96397351:CAA:C, 5:96397358:C:T, 5:96397377:C:A, 5:96397382:C:T, 5:96397390:AAC:A, 5:96397440:C:A, 5:96398887:GAAGT:G, 5:96398922:A:C, 5:96399952:C:A, 5:96399993:T:A, 5:96400071:G:A, 5:96400106:C:T, 5:96408221:A:C, 5:96408288:C:CA, 5:96408299:A:AG, 5:96408310:A:AG, 5:96408324:C:G, 5:96410771:T:TA, 5:96410797:C:A, 5:96410840:G:T, 5:96410844:CAGGGGGAT:C, 5:96410910:T:TC, 5:96410935:G:GC, 5:96410974:TC:T, 5:96410983:T:TC, 5:96412437:C:CA, 5:96412454:A:AT, 5:96416067:G:T, 5:96416072:T:A, 5:96421879:CT:C, 5:96421881:T:A, 5:96421893:TG:T, 5:96421940:TA:T, 5:96421951:TG:T, 5:96421954:AT:A, 5:96423354:CA:C, 5:96423401:CAA:C, 5:96425819:C:G, 5:96425827:C:T, 5:96425843:TG:T, 5:96425891:T:A, 5:96425892:AC:A, 5:96429256:CT:C, 5:96429260:G:A, 5:96429269:TG:T, 5:96429310:G:T, 5:96429319:T:C, 5:96432082:C:T, 5:96432861:A:C, 5:96432869:CA:C, 5:96432913:C:A, 5:96432957:CT:C, 5:96432971:AC:A, 5:96432981:G:GCA, 5:96432993:CAA:C, and/or 5:96433040:C:T according to the GRCh38/hg38 human genome assembly; and
wherein the MC4R agonist is setmelanotide, a peptide comprising the amino acid sequence His-Phe-Arg-Trp (SEQ ID NO:32), 1,2,3R,4-tetrahydroisoquinoline-3-carboxylic acid, or ALB-127158(a).


SEQUENCE LISTING (32 sequences)

SEQ ID NO: 1 - DNA, 42916 bp, Homo sapiens
  source 1..42916: mol_type = genomic DNA
actcagcctggagaccgaagcgcttcactgagcgctcgccgccgcccagcctctcctctcgcgcctcctagctcttcgcagagcaaccaggagccaggagtggtctagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcaggtaagagtttccactttcaagaaactttctggggccccaggggactggcgggactggagcgcggtcttccaagtgggagccccaggttgcactctccagagcccagagtagcaaagagcaaggagctgctccagccctcgcccctgtcgcgcttggagagagagaaagcttgcattatttactgtttaggctgggcgcagccaagcagaaggaagccgctgcagctgggtagggcttatgcttactgttggccgctctagaattgaaatccgttcccgcgcggtttggaagaaaaatccctccacgcttgttcagggaacttgagatacactcaagctcttaccaaccaagcgcccccgggagaagcagtgcgctccatcccccgggactgaggcggtctgcgtcaggagccggctgttgccttcttttcaatatggcagcatcagatccaagtcctgatgcaaggggctaagcaggcttggggctgtccgccctggcagcctgtaagggcacgcccagggcattggaagagtgccagctgctgggactcgcgaccaggaggttgggaggccgcggagtagcctgggaagctggggggagaaatggtgggtgaaagacacgggaccatctatccccgcgactctctagggagctccggcttttcactgggaacttataaagtccaagcttcataactttgcccggccagtcccactgcagggtcgagatgccgaactgactatggggaagggatctatttctttcttgttcttcagccaagtacgtaatcgttttcccgggaatttcagcttattgttgcctgtccaagtgaaggcttggtcgataggtagctggttggaggtgggtgggcgtcaagacattaagtgggagactgcttatagctgagcgattccgaggggaccctagggagtgtgacttctagtctagagctttcctttccttggctcggaatcaagccctcgccccctgcttgggaagttggtctgcattgaagcgtgggaggaggcagttgtaaaacattttggcgcgttttttctggaggtggtggttgagacgccgctggagtcacggatgagtaatcggcgggggcgttggcgattctgtagaccgaaccctctggggaattcgcctgctcttgctagaggcttcattctcaactttataaaccaaattgtctttgaaatgcccgaaaggaaaatcaatgcagattggcttcctggtgtcccagcagcagatgagtcttttgcttttggccagcccttgccctcgtgccaggctccgtgttaggggctgaggaataagaggagggaaagaaacgctctcaagtaatcaagaatctagaggagactagcttagagataaatgattgcaactgattgtaatttgtgtaatcatagaagttgtacttgaagtctcaatattcattggttgaaggaatgaaagtagtagggaattcgggaggggtgaatgaaaactgcccaaaggagaaaacatttgctccgggaacagaagtcaaaggttgccaggagcacataatgagatcggagaagacagtctaggcatagaaaacagcttgtgggaaaacagtgagacataatacagtttagtagtaagtctgggaattgcacgcaattattaggaatttttgagacccaaagagggtatgagtagcagaggaaattgtgctggtgggtactgtggacatcatacaaatcatgtatgacctatgggggtggacactggggatttaattgtgtattccttgagcagctactgaaaggctaaatagcactggtaaccgtgtaggaggagactggggaagacagcaaaaactaaatctctcttctttttcccacctgttagctctaggcatgcatctaaacctagaccattcaatgtgaaaactgcttttcaaaataaataaacataaatcttaatagtaaaaaacaatacatcatcattcttgggaggatctttgagtaagggatagcatagtcacaaaagcattaacaaatagtgggggaaaaccatctatatttatatatgatttacaatactatctttcctctaaccatttttagttaagatggcattactaacaaaagttttaaaacaaacttaacaaaattattttattagaataataattctttgtagaaggctgatagcacgttcctggttctgcttatttctctttttgtttaatattactaaaattacactaatttagcattactatattgggtaccttattgcctcaggataaagcttggattaaggtagtgacaagaagaaaaaccagttgacagcagagaaaacagagctaagcactggtgatctttttctgcggctgctgatttctctccccagttccatccacagcttataggaagattcactgagattcaggcttggagcagggcctaagcttctctattttaaaattctctgggcgatgagaatcaccaatttaaagaacacgtttcttctgtaatatactgttgtcaactgtttgtttcttaattttaaaaatgcagggttacctagaggatttggtagcctcaaatttagtactgagtaaagggagactagctcaaaagaataatgaatatagtcctaaatgaaccaaagatgtcaatagatacccggagaagtacaaaccctctaaagtgggacaactgccctaaatgtagcatgttcttttctgaaagccttgaattagtggaaaatcaaacgatcactaatagtggctaatagcaatttatcactatgaattcaatggctacattttataacaacgataatttattttagtggaaagtggcactgaccatagatagtatcccaggtgtttgcctgcttaccacaaataattgttctttgaacagatatatttgattaaatgagcattttaatatgatattgtcacacatgaaaaacatgtatttcctccaatgtgtgtaaaatagaagcatcactagcctcctactgtgagctaatgaatatactgtgaactcaagcaataatattctgagggtcatcttaagacctctatcagtgccttcttcatgaagaatgtggatggagctggaggccattattgttggcaaactaacacaggaacagaaaaccgaataccgcatgttctcactcacaagtgagagctaaatgatgagaacacgtaaatacatagaggggaacaacatatactggggccttttggagggtggagggtgggagaagagaagggatcagaaaaaaataactaatgggtattgggcttaatacctgggtgatgaaataatctgtacaaaaataccccgtgacacaagtttacctatgtaacaaacctgtacttgtactgctgaacttaaaagtttaaaaaaagaaaaaaaatattaatttcagataccaggaatgggcttaataaaagttgcctatctctaagttagctgagttttaaactagtccatatatacttgtttgggaacgtggatatttattttatttctttccttttatagattggttcacttgaaaatcactacttattcaaacataaaaaccaccccagaaggtctcgaaggagtgcctttcatatcactaagagattatctgatgatgatcgtgtaagtgttgtacatttgtcttcaaaccaatactctagcttatctgctttctttatgatttctaataaaaacagaagcattttgcaaatgtggttttttttttattgtgaccaagattgtttatctgtattatttctaatttctttccataaaattataatggcacataataataacaagattgggttgaattggttgagccaaagttaattttacacatatatacgcagtttaattatataattacatatatatctaaagttaattttatatgtacactcacaaattcagtctaagagcatgaaagtgaatatttaaaatgagagggcagcactaataaaaatgtttgacaatacagtcaatcaacaaccttttattaggtacctattatgtaccaaacactttaataccaatgtgggctcaaagaaatacatgggccttactttcaataacatttcagtataactgaaaaaaaagtctattatttctatttggttttgctgtataaggagctagtgtttatgagagaactgtaaatccatgtcttcatgaggcaaatgtttccataagtaatctattgttggatccaaaccatctgatctcttttcatcctagtttgtatcttggttcaatgattaggttacagcagaaataaatataggtaaataaggtagaggcagtttgaatagatagctaatgtacctattgattttgaagtctattttcaaggtcaacaaacatattagaaaatgtttactatgttgggagtattgtaattttcatacagtgttgccttttctcaaagaaatgccatactggaagaaatttatcctgattcgcctgatatctgtgtacataatttttgttacctaggacaacctggagaattagaatgagagatcctcttggtgagaggattaaaaacatcctttcaacttcccagggggaaaatatatatatatgtatgtgacagaaaggtgatagaactgttgaaagtagacgaaattcatgaggactgtaagataatttgcccttttattcattctgaaactgatagaatttttaaaagaagaagaaaaagacaaattatgagggtagggaatgggaatggcctgggtcaccagtgttcgaatgtaaaagaaacaacaacatcctgacaatgctgtattaggaccacggacagtgaccgtatagctcatctacacccactgctagctcttttttgtcaagttattgcttgatgtgggagaccaaatcagaggccctggaccttattacagaagtttcttgtccactgaggtcttcccttactcacttcaagggagagggctcatcgtgtgtgggctctgaaaagcagaataacttctagcactctggctaggcttagtccccgggctaagcagaagagagatttccttctttgcctaaatgcatgcccaaactgcatgcccattaatgcacatagaactcaaactgacctgccttctcactttcataggagaaaattggacagaatagagcatacctaactgttattcttactcaaagtatctcatgacaatgaagatggcccattctactctttatacatccccttgatagctgatttggtgggggaggggtggaatctcaagacattatgaaaaaatgctaagaattccttctccagcacaggtataactttgtaaaaaattagcccaagaataattttattttaggaaacattctggaaccatagaaacttgcattggaaggttgaattagatgatcttcaatgctctttccagatcaaaagtattatttataattaagtacatgatacctataatgcataatagaaagacactaccaattttcctctataccattcagcctgctcttctagagattaatacatgattctgagttaatgacattaatcataacactgttttctacaattggatgtttatctgatcagacggcagagtgcctcctcttatatcccatttccaaaaaattgctcttggttttcattttattgctcttttttgatggaggataatattctttcaaatcagccctgtgaaatgcagcaacagtctatctttacaagtctgttaataaaatctccaagtgtaagtgaccctctacctcaaagattagtacttattccctagcagtcagcttccagagatcaactcagatgataggcagcattccaagcattgaggtattttaagccaaaacaattaagcttgaaaatacaagcactttaaaaataaatcatagtgactaatttctagctttcaggttcttcccattatttttaataggtttgcagtatatttttaattgaaaattgagggcagtcttagtggatggttttatccttcagttgcaaattatttttcggtttttatttttggagctgattcatgagtactctcacattaacaatattccaccacacactgaattgtcatcatgcatacagcagaaaagatgtgtttggggtaggaaatagtcatattgccaagaaaaataagagtatagaattatctcataatgaatataactacaaggcagcccctcactctctcagggcttcaagtataatagtattatgctgtaaatatggggcaggtaattttcgtattgtcagatctctttgcataatacatgagactgaattaatggtaagtgtgcgtggtcagtagtgctcattcatcacccaggatctgtgtctcatattaaatacttttaagcagaggtcgaagcacaggggagggagtttcaaatttgcatggatacagcaccattcttactttataaaatatttgcaatttctacctgttgtatgtaaagacagagaaattaatctataaaagcattataacagcagtctcttggcttgttgactcaatccataaaagcagacaaattttcagccatggagccctgaaacctcaatataaaaatgagtattatggtccatttgctgatgaaagcagactgaacttttgcttccactgtaaccacaatgtaaataatttagggaattatcatttcactgactgcttctcctccccctaaaggtgcactttactgtaatagaggaaatattcatctccccagtgggcaatcccttcttctcactgaggtgccagtttaatctgtagtcaaatgaaaatattgcagagctgcctgaactctgatgggaagggtactggagatagttagaggaagtatacagaggtagatatttgacttttgattttcttgctataaaaatccttgtaggtgatatgggctgaacaacagtatgaaaaagaaagaagtaaacgttcagctctaagggactcagcactaaatctcttcaatgatcccatgtggaatcagcaatggtacttggtaagtacctacagaaggactatgtgggtgggacctgggacctggcttctttatgttgttacatttgcaacatggatttttttttttttttatgatggagaatttcatagggagctatattgtctggagattagggctgatgggatcttcttgatctgcctcttctgttttgctgagtaaggatgttggaaacagatatctttagctgctgtgaaaaaaataggttgtattttttgtcttcttaagtttatttgggataaatgcagaaaaataatatcttgcaggttagggtttccaaaaacagtacttgtggcaaaaatctcatttggttaaagttattcttcagaaccctttaaagcactcacagagtatagtgaacataatttggcatatacaaccctgaatgacaattcttatatgacacagatgagaatctctgaaagaggttaaaagaaggaacacaagaaaagtctattgcaaatgtctagacttgcaaaccattctacctttaagatagttaccaaatccttgagcgaagaaattgttaagtgttgctgtctacctgcagggtgtcctctcttctgtttaatgttacgtctgtatggtagtagtataggctaatagaaggatttgttgttgagaattttataaaagattatttctcttgaaaaacacaagctaagactttgatactcttgttgcataagggttagaaaaggggatcagtttctcttgaccctacgttttctttctttctttctttctttctttctttctttctttctttctttctttctttctttctttcttttctttctttctctttctttctttctttctatctttctttttttttttgacagagttttgctcttgttgcccaggctggagtgcaatggcatgatctcagctcactgcaatctctgtctcctggcttcaagcgattctcctgcctcagcctctggagtagcttggattacagatgcccgccaccatgcccagctaatttttgtatttttaatagagatggggttttgtcacattggtcagtctggtctcgaactcctgacctcaggtgatccacccgccttggcctcccaaagtgctgggattacaggcatgagccaccgcaccgggcctcttgccactatcttaagtatttgctcattgcagcagaagagacttatcagttaacacttgtgcatttcctgtgtgacaaacactattctaaaatgtttgatatgaattaattcattttgttctcatgattgtctgtgttgtagaaatagtaattatgcccatttaacagatggaaaaacagagatattggaaagtagaataatttgccaaaatccatggctgctagtggtgggactataattcacgactccagagtccatactcttaacaactacagtctaaaattgttcctgcaactgacatagaggccttcttctctgactttggccaacaatctgtgtacagttccagcctcctgtgattcagcttcataccagcctggcaaatacttgtcaccccaccccccaacctctttatggggaggagggcacaaaagcaagagggcagctccttcactgacacttttgattgtggttatttatagatttcttattgaccctttcaccgggtacctggcattcttttactgtctctgtctcacatgcttagaaatgtgcttatatctgtgttcccttgctaccctgtacttatctcctgtacagtcccacactctgactatcagaaaatgtattttaaaaggaaaaccctgaatctcctctcacagacacttaccccctaaatcactcatacttaaaaataggcaatcacattgcaattcctgaggcaatcacagtctcctgagcattccaggttctgctttttcctttagaataaaaattctaaaacattcatgaatgactatgcaccaggcaatataatgagagctcaatgtggcaatggagaatcaggatttggggggagatgaaaacatcgactatcaaatagagcttccatagatgaaagggttgagcatgttattataaatgaaagaaatttagggataaaattcttcaaagccatgtagttcaaagagaaggagcacaggtgattcatcttttcaagtgatgtccatttgggacaatcgggagaagagcagcactctgggggttcatttggccttactctcaactttgttttctcatttcactgaattggcttcttctaaagagtagagtgacccaaggaaaaagaaagatgggtctccaggttggaactgaagtgcccattttgtagcaagcaaatgataattttatcaatgcttcgttgtttttctttaccagcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgtaagtgtgtgccttctcatgactgtgacttagggagctgtctgtgcagacacacagttagggctttccttcttttcaaggggatgctgctctggtgtcagtccctgggattatgggccaaggttatgtcccttctcccacttcccactagcttatatgaatttattttgtaccagtgggcattgaaataattcagtgggatcaaaaattgtacaaaagtatatgtgttgggagaagaaggggcagtgcctacaccaattaagttaccaaaatctgatgactttgagttgctcagtgtttggggcattaaagtaatcaaatcaagccccttgcctgagatcattgtcacagtttccctcctcacctcttttatgagtctatatccctgacattgtaaaattatatgaatgtgattccttccatgcaaatccaaagtgaataccacacaatgcacctatgccaagctgagaaaggaaaagttttctagaaatgggaaaataaataaataaaaacaatttgcttcattgtttattaagtttataatataatatgaacaccatcatatatcttaggtctttagatcaggaggctggtgtttcatctttcagattatcccttgtttttctctcatcctttccacctatcaaacattccccagctataacaccaagctaaagagccaaggaaaagagaaggaaaaatagtagcacagagtagaaattaggaatgattgaaatatacggaaagcattgaaaagtaacagtaagggtatgcgggaggcgtttaagtgtagctattgggttggaaaagggagtggagcctggaagcttgtcttgaatttcaagcaccctctctcaaattaaattacatgtttatttagtgtgcctgggaggctgaaggagagcaccatggacctgagtcccaccctcagccacttctttgcacctatattcataagagagatgagtgtcaaagtgtcactaactagctttatggccttagggaagtcatttaacttctctgggcctcaattttctcatctgaaaactgagatgactgaactaagtaaactaattccttccaactccaatatcccatgagtgcctgtgaaccataggcttggtaacattcactgagaattgaatgctggctcacctggctgtgttcatcatctctgcctggtttgtgagtaaaataaatattcagtgactcggccagaaactctaaaatgcaggcttttgagctatcagccaggatgggaaggcatagggtaagaggcttgggaagtgatgcttttttttttttttttttttttttttaatgctgccacagtgttatattttgttgtctctttaggatccagaggctagctatgattttaatgataatgaccatgatccatttccccgatatgatcccacaaacgagaacaagtgagtaaatatgcatttgtgtgaaataaagtactttacaatgcttttatcttgtttgagaaaattataccatatatttcatccacattccagtgctcaggaaatgttaatgcacaacatccataccacaatattttgttgcttttaaaacagaaaccaaagtttctatttttatattccctttggtttatagaaccatattttcattaaacttcttacattatgaaattctcctctcaaatgttactgaactgttgtagctttagaaagaatgtttgagacataaagagtacacactttgagcccatccctcccctcaattcctctatgaatcttttactcttagcaacatgctttatgttagtcatgaaaagaaatatatgtaaagaaatggatatgaacaatgcccacaacttctgacggtggctacagtcaccttcaccagttctctcagaagagcatggccaaactccagacagagaaatgcctctgaggtctggcccctgaatccaacacagtctccagggcattgagccagccggaagtctcctccagagttcctgctgctataaggccataggaaagagagaggagacagcacccgactcccagattaactctctgtcccttgaagccacattatggtgagagaggccaagagccacagaggccaagagcaagatcttctaaagggagaggtgaaagaaagacccagaggcaagagactttcaacttctctcctagccccaaagaaacttcacgctctaggcatgtggaccctcaaagtgtccatgcccatgggagagaccatctttgctcataccgatgttctcacagacatcaaggagacactcaaactttccttttcattttctctacacagaagagacttgcaccaaaatgatcaaagccaaggtcaaaattagggcttgttgctacaagtgaggatggaaaaagccaagcatctaacagctcttggtatatgtcacatgaagcgcttctttattctctctggatggcactgtctcttggactgttacccaacttgggtcagacaagtctgtctctgtctctctctctctctctctctctttctctctctctctccctctcttccttcctccctccctccctccctcccttccttattctccctcagaactcccaagtggctggtgcagtaatactgatctaaagttatttctttctttggatagtcttccctccagaaagccaacaaaccccaggatgggaggctggggtacatggacttggcctcaacttctgggtgtttatttcttccttggggaaaatgggaggcttgtggatagttcagggatatgacttctttccgtaccatttatttctttgagggtgactaaaggggttaaccgcaaatatcccttagaggtgaaaacattccagtaaaaacacaataggaaccactggcaagggtacctttggagcaaagtgggagtgtttcgagagcttacagcagaccctcagacaaactttttgctatatgtctgaagcaggataatgcaggaccagtaaggacatctactcttttggggcacagccttgcaatgtagttatggctttgccaattcccagagtaaagggtgaccttggagaaatcacttacctctctctgcctctattccattttcttcccctaaactgggtgacaacgtaccttctccctatgtttcttaaatgaacttcctgaggaaatggctaataagcacttggaataataagccttcttagagtggagtagaaacaactttcattccacccccttggttaagactacgggaaatctgttcccagacacttgaggagacttagatggaggcactggctccttcctaggcagtatacggtccttccaggcagatcctgggtctggccccaggatccagtgtgggtgataacttgtacgatggctgcatagtgggcgaattcttgatgaaatgacattctctggagtttttccagtcaggagatggaacactcagaccttcctcaaaacaaaggggatctcacagcacagcatttataatagttgttttatatatatgaatataataattaaaataaaatataatttccctgactcagggagttacctgcaaaggccctagggaagcagcccatataatgctatttgttttgtgcctgaaagccctgagcttccagatcatagcctgctgtgaatcttccacacagcattgagagagcatgtcatagcccaacagttaagagcagacttgggagccaaatttaggtcaaattttagatctgccacttagtagttgggtgatcttgaggaaattatttaacttctttgtgcttaagttttatcatcttttgaaaaagaatctctacttccttgagttagaggtattaagtgagtgtatatatatatagagagagagagggagagagagagagagagagaggtattaagtgagtttatatatatatatataataagtattatataacatatataatacttcattgagttaggggttaaagtgaggttttatatatatatatatatctcacttaatatatatatacatataaactcatttaatacctctaacttaatgaggtagattatttatctatatctatatctgtatatctggcatttaaacaaattgatttgtttaaaaattattccacaagatatttaaatactacccttattctggttaaaatattcttgtcacacttgttatttaagccatatatgtatatgtgtgcatatgtatatatatggcttaaataacatgtgtgacaaggatatttggcttggacatacacatatatatggcttggacactggtacacaaaaaagttaagtaagtgtttgctgtggtgatgatgatgatgataatgatcacagcttctcatttagtaactgaggtagaccttctttcagtgagaaatcaacatgacaagaataatatcatccgatgggagcagcagagacaagtcaattttctgagccaattcagaatctgctatttggaaatgacataatgagcaactctctctcaggatcctcttgactatcaaccaaaaaggtggcatccttatcatttatcagaaggtataataaaaagtagatttgccttctagcatgttgtcagacataaagcccttaggtttagaaaatgaatggctccattttgtcttggcctttacagcaattggagaggtattataccccaattccaaggttattatctcttcaaactatcagtattgctgttgttctttattaccacgtaaatgagattaatagaagctacatttgaatgctactctgaaagtcaaaagaggaatcactaaataataaaatcttaacagtgcaggtaatgcttaccattaagctggcataaatctgtattcattatttcaacaccaattacagattgtttttctagttaaataacaaatgtgacaaggatattttaaccagaataagggtagtatttaaactatctcatggaataatttttaaacaaatcattttgtttaaatgccagttactaccattgttctaagataatagaaagttctttactgcagcacctcacttagaaaagaaaaagagctgcggcctataaagaatgacataggaacacttttctttctctcaacgtttggaggtggaacctgaggataagaggaggaaaacatgtcaatgcatatttgtaccatgatcctcctgcagctctcactagagaatagtcgtccatatacaaggagagataaataataggtgctgtcacactgcatcattttttaggacaaaggcacaaacctggggatgtctgtcagcagattgattctgttggctcaggctgaagacctgcactggattcttttgctgacctgagccttgtctcccatcctgtatacatggaaatatataagaaggcagtggtgacttctggaagtacatttctcagagatgacaactgaattactccttctactaaggcaaagagcgttcatccatccacaaagcataagttgccttctgtatgttcagatgtgtgcacttttctcagagaatggtgtggtcaggacagcttcagttgcaaatgaaagaatcctaactcaaagattaagtaacaaagggattttaaaaatattcacccaagtgatgttctagtttctgatgtttcaagatccaagagctcaaacaatatgttctcaggacaccaagcactctgtttccatctctagtgctgctcgcctctgcttggcttcactttcaagagaatcctgcctggcagagcaagacccatatgactcacatggaacttaaagctcataattctcaagaaagagtggggtttcttccctataatctacatcagtccttgaaagttgtctgagaggaggggccccatgattgaaaaaaatcacaaaaagtcaggagggctgcttccccaaacaggaaaatgctagcatacataaaagaagcaggtgtttgctgcatcaggaaagatcttgaattaaagataattatattgtcctgacttagaattcctagccaggattaacatgaaggagcaggaatcttattttgtggaaggagaccaggaaatcatctttgctacttcaggaaaaagagaagttgaacaattcttcttgatggccagtcaatttaggaattttatattccatgctatgctgaattaatagatggccagtatagtagaattgaaatgtaactggccaacattattgctccgagctaacccttcctctctttcactctatgttggtatatctggtaggctcctccaagccaccaacagccttcacactcttacaggttgtttccacaatagaataaaatcacgaaataaatgaattcacattaatgtgcttttggcttgttggccctcctgagtcaggatggtggtgggaaaagacaattatcatcctcagaatttcagaatagatgctttagtggtggaatttggtgtttggaagacgcatttttcttccaaatactctgaaatcataaattaccctctttgcttgactgcaaaaccaatcatgttcaggcttctggccccagttgggatttcaggagcatcttggctttccaattagctggtaatgtcttgcaaatatctcaagctcctcttggtaccagataatcttcagacattttagtgtggcatatatttagaggagaggaggagggcttaataggggacttgttctaggatatcctactaaggctgatcaaggataatctaatccaaggcagtcactagggagaaaagtgaggagccactttgctaccaaaaagtacaaagcagaagccatttctctgaaattgtttctgaatgtcagagttcttataatagtatcaatagttaacactcactgtgttcttactatctgccatccactgctctaagtgttttacatataatttgtcatgtgaccaagcctttgcagacaagtaacttacccaaggtcacacccttagtgaatggcagaagccagaatttgaaatcacatactttgtctctaatacttgtgcccttcatctgaacataagttttatgttatactaatttaaaacaaacaaacaaaaacagtaacaaggccgacaaaaagttaatatacctatgccccattaattcattcatatgcaaaacaaatgtttgttcttctgtatgttttataattcttatttttcaatcctcagacacgggaccagatgtgcaggagaaattgccatgcaagcaaataatcacaaatgcggggttggagttgcatacaattccaaagttggaggtaaaacagaggattgtccctatagcttggcattttaatgtgaagcttacatcacccattgtaaatgggggaggggagcttttctttttaacaactggaggaacaaattagttccatgccaaagttgctacaggacttgaatatgatttgccagcgagtcaatagatatggattcctagaaaatggatagttgaatatagcctatacatctttgcaggaaaaaaaaatgtggaaatgttgaatagcatttgaaccaaatctaattttctctatgttctgatttagaatggtttgttattgagtgcttgctaaggaaacagaaggaggaggtaaatttaggcaaagacttttcctgcctctcaattctgtagaagttcttcactttatagagaagaacagcttcaaaatttaaaaaaagtacatacttattatattaaatagccaaattttatcttttatagcaaggatgtaggatttaattgttttcatcagtgtccttatggggaaacacatggtaagttgcattagggcaactgtggacattttaataaagaaacgatttacaagaattagagaaaaccaacaaggggctaagaagtggtaggcagatgttatcacctttagcctcaaggagcaaagggaagaagtcagcagaaaatagagagggaaagctgtggtcacaggagagagccactggataggccttgagtacaacaacagagccacttctaggcaaaggggattcaggggtataggcacctcattctcctcttgattcccaccctcttgctggtgtcttcagttggctgaacccaatgggaagacagagcccattgatgcagtccatgttctaatggagaagagtggaaagtgtatctgcaggggccaagagaaaagaaaaaactgtctctcctctttatgctatcccaacctcaggctttcatgttatgttataagcttcaagtttagtttggttttcaaaaaccataatcacaaaaagaatcaattgtagaacatttcaattacaataaaaccccattaaactctacatgagtataccttgcatttggttatgcatttctttttagaaaccaaataaatatacatcgtgacatgtaatgcatctttcatgaatatagggcactcttagaattttaagtgtcattcctgattcctttgatactaacatcttcaaagtgttctgttaatttttaaaaaaaatgaagggagaagcacaaattatgtaattagctagtaaaagaattcactcttttagtcataatgaacgaaacagctatagtttgtgaaagaatcatctgatcccttcattttgtagagaaagaatcagaagcaagctgctcaaggtaatgaaagcaagtttgtggcagtgtagaactgaaacacaagattccaagcctactgatgttttccattactccatcctttgatggtctggccccaaatgcattttttaccaataatcttcatttattaggcacctattgtatgtcagttactctacaaatattataggcagtgtatcctggtggttaacagatttaggggctagatttgtagggttcaaattctggctccccagtttcaagctatgtggccttgagcaagtcactatcccttcctggcctcaatctctccatttataaagtgaggagaattacggcacccacctcaaatggtggttgtaaggtttagatgagtttatacatgtaaagcctgtggattggtggctgcatatagactatcatgaatgcacatgagctgttattatcatccctattttacagttgaagaaactgaggcacagagggttaactggcttgcccaagattccacagctggtaagagcagaactgggattttttttttttttttttagacagagtctcactctgtcgcccaggctggagtgcagtggcgcgatctcggctcactgcaagctccgcctcccgggttcatgccattctcctgcctcagcctcctgagtagctaggactacaggaacccgccactacgcccggctaattttttttttttttttttttttttttgtatttttagtggagacagggtttcatcatgttagccaggatggtctcgatctcttgacctcgtgatccgcccgcctcggcctcccaaagtgctgggattacaggcgtgagccaccgcacctggccgattttttttctaaatgcattttttttttttttttttgagacagagtcttgctctgttgcccaggctggagtgcagtggttgcaatcttagctcactgcaacctctgcctcccaggttcaagcatttcttgtgcctcagcctcctgagtagctgggattataggtgcgtgccagcatgcctgactaatttttgtatttttagtaaagatggggtttctccatgttggccaggctggtcttgaactcctggcctcaagtgatccacccacctcggcctcccaaagtgttggtattaaaggcatgagccactgcgcctggctctaaatgcatttttgaacaatttcttttttacaaatacaactttcactgatcatatcaatttcatatcattttacctttgctccatagtaaataagtgtgaaaatctaaggctgtgcactttattgtattatgagcctgaagccagaaataaccaggtagctgcctgtaatctgtccagattgtttttgccttcctggctcttgaattcataagtctgatgacgggtgacagcctgttggaaagcacaaagagctaccgcacctacattgtcctgatgaaaaatgattccccttgggggtgaaatccatggcaatgatttcatccttgagattatttagaagaataggaagcatgttccttgaaaatgtttcttagcaaacacatgaaaggatggcctctgggttatttttaatcagggcttagaaacaaaagtgctttccctatttgttagtttcctgttgtgactagatcttttgaagctacatgtttcctgtccagagtccttgtgtggcgtagactgggccacagccatttgtgggacagccatgaggaggtgcatcaccagagggctgcttggagggtctttctgaaagctggtctgtcatttagttggggtgggagggagggcaaacattttgtaattgacaacaatcacggccacttcctctcatccaaggatgctgctccacttcagactcaatgcccagagcttttgctgggaagagagccttgagtgggaccaaaaaaaaaaaaaaaaaaaaaaacatcacagctgcctacacagtgcatggtattgcatagtattatctctaaagtgcgacaaaaaagcttatgggggcagcaacatgttccttccttctgtggtgtcagtagctgggttacagaaattggggacatctatctagtagttacctggtttttgactcttaaaatcctttaaaaatacaaatagtaagagtattgggcttttgtccatgtacatactgacatcaacctttgtgtgtctttgtttattttgtttctcaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggtaagatgctttacacagacccacatgaaatgaaacctgaccatctggatatgaggagaaggaaagaacacttaggttacatggaataacattgttgtggatttcttaacaaagtcctttacaattttaaatgcacttagagggccaaatcattcttcagagggatgggagttaaggcttaagaaagttttccctatttgtggccaggcgcgatggccaagggaggcagatcaattgaggccaggagtggccaatatggagaaaccccacctctactaaaaacacaaaaattagccaggtgtaactaggttgcactgttctgcaacctagtttagacagtgaaactgtggtctcaacttagatgcctatagtcccagctactcgggaggctgaggcacaagaattgcttgaacccagaaggcggaggtagcagtgagctgagattgcagcattgcactccagcctgggtgacagagtgagaccctgcctcaaaactaaatataaataaataaaatgaattttccctatttgggtctgaggttgaatggaaatttattacttttagcatatttattttaaaagtgcttctggacatttctttctccttactcattttcctgctgcttttgggtttttacactagtaaaaaactactcagtcttcttccaagcgcggaaatgcttttcaaatatagaaacgtgttgccttctgctctgcacgtgcctgttgaatacctgtgccttggttggattgctctgtcacttgggtttactgtattcttgcacagattctaaagccttgtgctccataagatgaaacaaaagtagaaactctggagagcacaaattgaaaaatcatagaaagggcggctttgacgagttccattcaattgaagagtcccttttaatgcagctgcaaggcagatctgaataattcctaattttgtcattctatcagtcagccagaagattgaagcagaaagaaagagaggaagttcaaagatttaagtgcagtgacataaactaagagaagggtggaaaaatatgagcaagtgcaaaacgctagattatgaatctgcataagaaagaaaagcgtgtgctcagccctaccccaggagggcccagagactggcagggccagagtccagtggcttataatacacagccttgtgagatttccaatatcgaagccaaaagatctggaattgaaaatagatcccaaatagctgtctctgagaatttcacacatgtagtcgttggggattttattgggatataacaatgaccccaatagatgataacagataatatgcattctggcttttcctttaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcgtatgttgcttttgtctaattctctctgcttagttaggagagcgtggcatgcatgtgaaatgattcccctaactgacctttggtacgactgattcgcttaagcttgatgtctcagttgggagaactcacacttactcacttgaaaggctggcatccatcagaaagttttctctccagggataatgacacttctgccaagatgatcttcccctctgtcatttttaaacccagccttgagaagccaccaaacctgtcattacacacagcggagactccaaatgtcttttctgcttgtctgatgcctccctggaaacagccttaaaggagatctcagggttgtaggggctttatcacccttgtacttgctgatcctttaacagcagggatatagtctgacctgccctgtcgcagatgataaaaatacagagtctacaccagctatgtctcctaagaatggatgacctgcaggaaaatagcagtttcagtgtcccctgaggtgaactgctcagcatctgggtaagcagctcggggaggtcgtatgctcagagagctcctccagcccacgcttgcctgcgcctttgcttccatgagtcagacggccaagggcaattactggatttcccttcaagggcatgacgatggaggggcatttaggggctttgagaacaaagggagacacctttaatttaaaaaatgtaaggcccccaatttaactcctgacacagaaaggaccaactagctaaagtatatagatcctatctcagtgaaatagcaggtcatgatctaattagagggtgggtgaaccctataataatgaccttggaatgcgtgcaagattcctattaatggcatctcaacaaaattgtcaacacgaacatcttcttcatacaggatcacttctgcaagctagtttagacagtaaagctgtggtctcaacttagaagtgtgggcctgcttctcacgcgcatctcttcagctactcaagtaatagacagtggcattctaccgaggggttttatggcaggcacattttaaagggtttgaaaggggcatatcagacatctgccccacttgtcttcagagcccctggctgttgtcatttccctgggtgtccagagctgcctttagaaaatggcctgttgggcatttcagaatcaccatttcacagactgttccttatcttgggtaacctgcactgaagcagaagaaagattttctatctttgtaaaactccctccttaagaaagccagccttaggagagacagctggtggattctatttgagacagtctcagtaacataacctttttcttacgcaaaccttttcctcagcatagtcatctctgggggcttgcttgcgtcaacaggtaaaaagggcaaccttgcttccgcagccctctgaggagcagagccatcataacttgagaaattcagcaactccaagcaggaagccaatggaggtgataatagaacaaattatttttttacacctccctcccacacatcgaaatctcgagctagaacttcggtgctggagcctgggtgggtgtcttgttagtaatcccttggcccaaccagcagcgccgtggtccttaaggcagacaggaagtcttttcagtttcttcccttcatctcagcaatctcttcattaccctaaagttagccagggtaattccttacttgcaagaaaattatgtaaaactactttcacagagaacatctcgggtaagtctcgcatatgagtcagaaaatataaatcgcattttacagatggggagccaaatgacttgcccgaggacaaatctattaagtagcagttagagaatttgaacccagaactaagtccagcacactttccattttaaagacccatcgttttcctcaaagtgtaaatgtggacaccaactcacaggtatcaaatctagttcaacagtgagtgcaaactacatattaagcattttttgtgcaagtagttcaaatggctaaggccaggatgtcttcatatccttgggctgaagaggcagccaaatgtcaatctgaaggcaaaagggaattttgtatgagacagaatttctaaatatgccagaccaaacacaatggacccgatgattaatgggccagcatgattcacagtgattttgaaactttgttttttatagacagcatttcgtttgggaatctcagaaaagaaaatattcgtagtgggagtacttcatccccagaaaaggcatcttgctgccagaattatagagaagttgcatatagagggtgaactaaaatatcagcagatggcaaacatccgcctgtggtttagaggagaaagtaggcaatcgaatcagctgagtttcctggtcatagcagatgcagcctttggtacagttaacccccaagacaggcccacactcctcacgtgttctccctgcctttctctccttcctgacgtcatccagacgagcgctgacctgcacaatgactgcacggagacgcacacaggcacctcggcctctgcacctctggctgctggcatcttcgctctggccctggaagcaaagtaaggcccagaaagcttctaatcacctttacaaaggctgattctccttatttttaataacacttttttttctgaatacatggttacaggagaaattttggaatactgcaaataaagataagctctttcctgccacttcgaggtcactaaccatagttaacatctcaatgtagatttaatacacacacatcttacaaaattgggatcttaatatagacgttattttgtaatctgtttttaaaacccattctacaatatattaaacatactttctcctgtctaaatcaatatgctttgacctttaaaactcatgctatccctcaaatcttcttcccactcagctacaacgccttttgtttattctcttccagcaggattttgtttaaatagtttccagtgggcagtattaggaacaagtttaaaaaactcctttctttgaatatgtaagcctagtcctggagctgatataaccacatcaggggacatgagatttaaaggcaggagttttatattttcacagtgttccattgtataaacacaaatttccttaaagtcatctaatcatgcttaaacaccattgtcagtaaatattagttgtcatctctgacaatttcctcaggataaacactcagaattggaatttctgggtcaaagggcaagcatgttttaatgatttttatatatattgacaagatgctgccttgaatgactataccattttatattccctccaggaatatatgtgtgaaagtgccagtttccaaaacccttgtcaaaactacttttattatcttttaaaatatttgctatgttgataggtgaaaatggtatctcattttaatatgcttgtatttaattactaatgaagctcaatatttcatacccttcagctctttgtcttctggggttgcttctttatggccttagttttgttttatggccttggttcacaaaagctagatgctttataagtttgacatttaccttttaatttgtttctatgtaatttttaatgaaatgtagttaatatgtatgtagttatgtttgtcagtctgtttctttaaggcttcttcctttgcttttatgctcagaaaaaatcttcacaaccccaaattagataaacattaattatatgtcttttattatggtttctttaccatatttaaattctaaatccctctggaatttattttagtgtatgatgttaaaaaaagatttaactttttttttctcataattacccaatagtccaatacttaacacagaggaaaaaaattgaactgtctcacactgcacatcacagctttttatgtgtcctcattcctgctttctgatgtgtggtgacagtgaatcttgaaatgtcaagtcaggtgctatggttgcatattagaagaagtgattaaacttgtaaattactattatccttcctaataaggagattatcctagaataaattacatattctctagagagagaacaaagcagtgtccttatgtcatattcagtagactcagttcgtaataacagaaaggaaaaatatcttggcctttgccatgactggaaaagccctgaggttcctgagcctttgtccaggagagtccagtttatctctaggatccctggggcttcctatggagatggggtatattcctgggtatctcggctgtgagatggctacagtggctaagcaaggaagtctagcaaggctcttaccaagagtccacctacttggaggggctggcaagatgtttggtaagggaagaagtaaattcaggtgctcaattagaaatagatattgggccctccagaaaagctatgttcagaatctaagttcacttataataggaagtagatggacaaaagacaaaaatctatgaacacaagctgagactggcatcagcagatccaaattaaccgagaaataattgtccaagcagtgaaaattgcaaggaacacctggcttcaagttctagctctaccactagctatgtggccttcggcaagttaatttacttgtggggttcagtctttccatctataaaatgaaggggtcaaacgagacctttttttacaaacttcttctcttcctaagattctcagagttgagattgaaaatgtcagcctgcataggtaatccaacatgaattgttggggtcagtgatgaggggtctggactggaagaaaatagggtcctatggattataggatcctgtgcaacagacttattatatttaagatttcaaaacacagactactaccccagggagctgttaaaagaaagcaatcttaaaattttatgtctagagcctttcttctctatccaacacctgtcagatgcatgtaaatataactagccgtgcctagcccatccttgtatagtgaccaatatagtagaaatatccatctgctgaacagaagtaatgccaattttatgtaagagctgaaattgtaaaagtgcaatccttggtttctaaaacctcaaggcttctttttctgtatagtggaaaatgatcctatacagaaaataatcctttccttttctttttcttttggagacagtgtctcactccgtcacccaggctggagtgcagtggcaagatctcggctcactgcaacctcaacccctccggttcaagaaattcttatgcctcagcctcctgagtagctgggattacaggcatgtgccaccatgcctggctaatttgatccttcttttttctatgtaggagaaaatgatctaggaatgatctagcctcattaatctcagtctctgatggacagtctctaaagcaggtgtttcatttctgtgaaacactcagatgggttccaaggggtccatgaatctctttgcaactaaatgcagaattgcttataaatgtatgtatggcatatttctggggaatggcttataggtttcaccagattctcagtgttcgtgatttaagagatattaagagtctatgatctcccaggtgcttatcataagaatttaattcccttcattttgactcaattcatcagtaagataagtatgactgatctatcttaacatttatatccttactgccaaggtacatagaggttattaagatttctgttctcaattgaacatttttctgaactctattcaattgtctataccaaagctgaatgccagaatttctagacatgtgaccaagctttatcccaaggcactcaagaatgagaaaatcatttaatcatgtgccatcccacaggaacaactatggagactgaagagcccttgcctaaacttatcaagaataatggcacccaatattgattaggttcttactgtagaccagacacagctggcctctttatatacaatattaattataacgcttacaacattcttatgacatagaaattattaatttcacctataaatgggaaaaaataaggcttagagaggttgagatatataaccaagatcccacaaagaaattcaaactcaagtctctctgactgcaaagtctgtgctatcagcaaccacaccttgcagcctcccacagccctgtgagggagaggcaatgttgaatggcagcctatcatctcctcaaataaaggagggttcggaagattgaggctgataaatgaacatgcatttatgagtttcttataagttttctgtcacaagcagactactcaatatgctgtgttaaatgttatatagccagagtattggggaatgggaggagaaaaggaaaggagaaggaatggtttctggctgaatatgcacaaagaaatactttccttccccaaagcgatgtttctcaagtaccagcatatatagtcatcacctggggagtttatttaacatgcaggttcccaggtcattttgccagcaactattcagaccttgtatctgcctaggagtttctggtattcaacctaggaattccttttttaacaaatctcccaggcactttttaatgctagtaataccttaaccaagttttgagaaacaaatgctataaagagttacttttaccactgtgttttcctacaacctcatccacctgtgtttgtgtagaaacccattagggtagttagtaggtgtatcttggagataaatatactaagagaagaaggaattccttgcataaatgtttgtggcactattattattatgtagtgagtatacctaagaaagctgtagaaaatagcctcagccttctgaggagagatcaaaagaatcagaataatttttaataggtgaggaaaattctgaagtgaaccattgaatcccttcacacctttggaaatattaaacaaacagataagccaaacttctggtcttattaaaacctggagaaagagaaatggactttcaatgctgcatgaaacctttggaaaaaaatcacaacaaaaaagtcttgacttagtgggttgtgaaatgttagaaagtccaagttagaaaaattttaagatctaggctaatttaggtatgattagaggctgaaccagatagctcctaagacttttcactctggaattttgtctccaaattacattacctacatctcttctaattagaagcaatgcttaattcggtgtttaattccatacccttattttctgtaaacatatctttgattagccactcaatgcagtgtttaaaaatttaattatcgtggatgtcattgagcacaaattaacaaaggaaggctgtcaggaatgagttgcactgttgttagcagctccttttcatttatgaaatacaagcacacgaatgaacaataactctgttagaaaaatacaccggggcctgtcatggggtggggtgagtggagagggatagcattaggagatatacctaatgtaaatgacgagttaacgggtgcagcacaccaacatggcacatgtgtacatatgtaacaaaactgcacgttgtgcacatgtaccctagaacttaaagtataataataataaaaaaagaaaaataggtttttctaattgcatgacaatgaagctccttaatattttgcagagactagataaaggaaatttttaaaacaaaataacctgcaactgcatataaaataaatgctctactacagaaacataggacacacatgctatatgtgataatcatgattatcagcaccttgccttaagcctcctcagtacgcttcctccttcctgcctccacacatgcacctgcacatactgatgatccaaacggaaaactggtgtgttgcccattgactgccttgataagaggcttttacctaaaactccttttaaaagactggaaggaagcaagcatttcaataaaatgtcctattctatttacaagtgtaccaggtgatcatgaggcaagcattctgggactcactgaggatccctaaactggtgtccttccaaggcctctctgtcagtcactctccagggtaaagtggtatgtttgtcgggagctcagctcaggtgagctgagctgtcaggtgtggagtagcctattaattcaacaagccaaaaatgaaagcattgagcctttagtcacttactgctatgacataagcaagagtctgaaaccggagaaaacacttactcgccctgttcctggcatactggttgagtggctcagcacagacttgagggtcatctcactgctgagggagccccgaacaaaaggctctggcaattttatggactctgaggtgtgaggcggggagggaccaagagagtacgggagtggagaagcaccaggcactgagtctgagtgaggaaagtgtctagatgtttcccctcctctctccacgaggaggcctgaccaagggacctaggaaggaaagctctcaccagctaggaatgtcaatatggaaagaacatgaggggccagggggcctgcggccttcatggaaactcctctcagaggctgcgagccacaaactgcacactgtgtctgcatggggagggcagcttccccgtgtgtcgtgccaggtaagacctttgaaatcgcctatggtcaggcctgaaaaatcacacacggtttttaactaggagccggactcttcagtgtttaggaatgtccactgcctctttcttgtcacaggggattactgagccagtacatgatgggctctggggagaaggcagcaaagtgagccagactcccctgatgattgttctccaaccgagtgctgagacaggaaccttgaggagtaaaaactgggcatctacttattacataatgcttgattggcttcagggaacaatacaggtctcaaagtcgaggaaaagtttgagaatctgactgatgactaggagcaatatgttttaaaattatatttggggctagttagcacttaggcatgttcagtctctgaaccaaccatatctgctgagcatatataccctgtgcccagcattcttctgggtgctacacatcagagaacaagatggataaagatctcaccacttctcatggttaaattagaaccagggagagagacatcaacaaaacacaatacaaatgagtttcccgcatgttagaagcctataagtgcaatggaaggagaatatgcacatatagcagaataaaagagctcgggaccatgtgggagagtggaaattttaaataggctgatcaaggaaggcctcatcatagtataatagatccaacaatcccttatatctctgatttatctttatagtgttgctaaattaatcttactaagacttatgattttactatttccttactcaaaaattctcactgattcccactgcccaaaagataaagtataagctccctttctggcatgccaggttctcatcgctctggcccaatactcctcactgcctctcccctgccccagcaaaccaggctgtcatctggccacacctgtactgtcccactttggtgcttttgctccagtggccttcttctctatctccacttcttaacactctaccccctgtcaaagtccagctcaaatatttctcccccacaaagttttcctagatctttctattccatggaaattctttctactctaaactcccacaatgcgttaagctatgaatatagcactcaccagatagaattataaaagccttaattctttttactggattgtaaacttcttttttttttttttttttttttttttgagacggagtcccgctctttagcccaggccggattgcagtggcacaatcttggctcactgcaagctccgcctcccaggttcacgccattctcctgcctcagcctcccgagtagctgggactacaggcgcccgccaccgcgcccggctaattttttgtatttttagtagagacggggtttcaccgtgttagccaagatggtctcgatctcctgaccttgtgatccgcccgcctcggcctcccaaagtgctgggattacaggcgtgagccaccgcgcccagcctggattgtaaacttctaaaagacaagaaccacatccttgaatatccccatggtattagcatagtgccttgcaagagtgagctcaaatagctaagagtttcttgacgcttttgatagataaagaaaaaggcaatacatgtttacagcaggtagccagtctgtgtctttaacatctcactcttcctaagaaggaagtcaaggcctcgtctgtgggttcataatgctgaaagtgctaaggcttttaatggacagcatgagactggagggcataaagaaagtgttaccatttaaaatgtttgctcagttaaatggaagcaattagaaattaaggacaaagttttatacttctatattgctgaggactgaattttactcatagtaaaagcttatatgctctggagagaagtcataacttatatgttacagggagaagttttcagctctgtattctgaattctgaatgaaaagatatatgaacagtaatagacactagaataacataactacaatagcaaaaggcacttcataatccctgaatggagatgcttattgttagcacttgcttttgcaaggaaacttcatttttctctgaaagacactttgggtcacttcccctccagcccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccaggtaagtatctcctggaattttaaacacatgtgtgcccatggcataatttcattactatgtttgccattctgcctgatttttttgtatctacccttctgaagtagatggaggtggaattttctagtgttctctgtgggtatccatggggactaactaagagaccaaagctctagaaaccatgttaaccatttgcatgttcctcttgtagtccacctaccacatagcacccagtctaagatgcaccattgttttattgtcaattaaactgtgatcaaccacccatcagttttaagattcattctaaattcaatgtttaactatttttttaaaatgcatcttagggttgatgaaatgcagtacttgccaaccagttttacccaaaatctgcttgcaaaatagccttgcttcatgccacagtgctgtgtgattcatggcttttcttccatgagggattctttatcatacacttacttagactccctctagcacaccacatcttgtcacagaagaaatttctactaggagtgttttgtaatttctatcaaagcaatcaaaataatactgaagacctgctcacacaaaatatccaaggatgggaggcttggggcatgccttcagaaggccttctagataaaatcatttttttcttaagaggggctttttgatgggtctacaataaatctgcctgaatttgactccacagcaaatagcattgtatttaagtattagtgccccttaccattttcttatgcattaaaattcatgtatacataattctgaattaggttctacacatcacagggagtgacaaaaagcttagttgagcatctagtcaagattgatgcatgcagatatgcataaaatgcaaggaagtttggatatactttattcaatgctgattctttaatgtatttagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggtaaattaaaaaagttctaagccatttatatttttaagtgtaagttgaatgattcaaataactgattgcttttgtttattcatagaacaaagtctcctaatagatattttcaggtcttaaaaaacatgcttattttttcctttttcttggtgttagatctccctctgccctgcccatttccactgatcctttgataagcagagaatgaggcccattcaaataatgctaattaatacacttttctgaagtcatgcattttcaaactgtttacttttccattttactgacctatcacttttctcttagatagcaaattagtgaaagttactctgataattaaccaaactcacagatatatccgaaacatacaaggagtccattgaaaactgggaattcagatcgattagaggagattgagaagattaacaaatttatcatagtaacatgaataaggccattttatatgatgtatgaacatttgctttttcctgcgagggcactaattcagataagatagtggatgggaaagagctcgtgaaccataagccataatctaaccaggaggagcagtcccaatactcctttatgttttagcaggactcaactgactaaatttggtttctgtgggtgatatgtgaggaaagatgagctatctctatgatcttcctcctgaatattaattcttagaggtttggaataggctctgttaattttgaagttatgctttctagatgtattggaattaacctgaaatattggtggaatgcttttttaattgatagacctgcagttatttaacccttccacatagaactattatcaattgatctagaaagggctgggacttaaaaatttacaggtaacctgtcttttaatataagagattgttaaattaatatatctttaagaaattaatacattaattaaataattatgattattaatgattgcataataaattaaataataatttttatgtttaaatcattatggagtcaggcacctactaaattttcaagatttcctctcacacttcagaaatgatagtttcattaaaatatttttagatttttgactctctaattcaatttgtaaatgtagttcgccgagttttccttatttaaataaaccttagtcaacattttcattcattcattcattattttttccacaatatttattgaggcaaataatattattcattattatatatgccaggcaatgtgctaagcactatagataaaaacatcatattttttccttgtgagtgtctctatcctggtcttatactaaaagagaaaattaggagaactcatctttttcagcttttattttcagttatcagatgctagagtgtatcctactatcagagctattaggacattcattaacttgtatttgtttcataggaactagcactgtgctcttggctgaaagagaacgggatacatctcctaatggctttaagaattgggacttcatgtctgttcacacatggggagagaaccctataggtacttggactttgagaattacagacatggtaagtgtgaatgagaggatgaaaaaacacaagcttacttttaaacattttgaagcacttttaaggttgatttcatcattaattagggccctaaagaattgtaccccttcttagtagaagtaagggaaaagtagagtgtttcaagccctgaccacttggttacgtagataatattcaaaaagaaagattaggcttcagccatactataagagggggaaaaacacttatataaacaatgtgacagactgtgggacaaatccaggtgttcccatgtctgcatgcagccagccatctaacctattcagccaacaaaatcaatcatattcacaaagatacttaaatgctcagaccaactttggtgtgacattctgccagagtcacatcttaaaggcatgcttatgattattctgcaatagctatttagatgtagcttaatgtttggagagatatcttatttaatatgacttttttaaacagttttagtcatgaaacaaaccgaaagcatttctcatttctttttcattctgtttactaacattctttgacatccttctgtaattttaacacacagtcacaaaaatgcaagtacagagcaaaagttagaaaaaagccaaatatagcttaattatttatttttgcatcatttctccaacattctctgtaggtgagaatataaatgaataatgttttggctctgagttcttctgtgacaaaagtgtaataaatatcagcagtgtagaattatacgcctaaataacttttttttttttttttgagacggagtcttgctctgtcacccaggctggagttgcagtggcacagtctcggctcactgcaacctctgcctcctgggttcaagcgcttctcctgcctcagcctcccgactagctggaattacaggtacccaccaccaagcctggctaatttttgtatttttagtagaaatggggtttcaccatgttggtcaggctggtctcgagctccttacctcaggtgatttacccacctcggcctcccaaagtggtgggattacaggtgtaagccaccacacccggcctagataactttttgaaaataaatcatttaaagttcttggagatgtcaattaaattaatacaaattctatagtcgtttgttacatttttttctctgaggatagctttctcctaaagactgctaaaattgtgatttagaaatggaagttgtaaaatgtccttctctcatagagcaaaaaaagaatgttcttagcatctgggcctgttcttgaatttggcctctattcttcaactcaatttcaccatcaggaaactaagaaaatctatttaccaaggtgggatttatttcatgtcaagtctgcaaaatctgaccttacacttctaaatttttaaagatattttagattttaacacattatttataattgttgagccttgaaaattatgactattcccccaaatatgcaactttttgtaactgaatttatagacctcttatttatttattttttgtcattgaactttatagacctcttttatttagttttttattatactttaagttctggagtacatgtacagagtgtgcaggtttgttacataggtatacacatgccatggtggtttgctgcacccatcaacccatcatctacattagatatttctcctaatgctatccctccccttgccccctactccctgacaggcccattgtgtgatgttcccctccctgtgcccatatgttcccactgttcaactcccacttatgagtgagaacattcagtgtttggttttctgttcctgtgttggtttgctgagaatgatggtttccagctttatccatgtccctgcagaggacatgaactcactcttttttatggctgcatagtattccacagtgcatatgtgccacacaatttttaagggcttattaatgctagaggttgtttataatactccatagaggctgacttttttcccagtggaatgtttctataagttgataattgggaaatatatttatagaaggtacagtcagctttccaagaacacatccaatatggaaaagaagacttacatagagatttctgatttttaaattaaatgtttgattatgaagcaatcagtttctaaactgttgaacctacaaattgtcaatttttatagcaagagtggatccaatggtttctttcacatgaaatgcacagaatcttaaaatgagattttaacaatgaaatcctttttagctattttgaatgagtttgttttatattatctaaaacacatactaaatgtaggtatgcttatttgttagtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggtctgtgtggctctgttcttggcatgctctcttttgaactggggtagacaatgtagtcagtgaagctgtacccaacttccaagcaggaaataagtcaaacatggatggggaggataagaagtgatagtggtaaactattcttttccataggataaaactgtatttttttcattcaatattttatccagaacttagaaatcacaattaatgaattcaaaagtattaaatttcctaacatgaattttctccacttatgacagataacaaatggaaaaaaaagttatctttatgcccaatcacatatatctaacatttgtactgtactttgtttttaagcattttccacattgtctcattaacctataatccctataacctaggaaggatagacattcgtgtatccattttgcagaaagaaaaacaaggctcaaatggccaaatcatgtaggcttctttttattaattcattaactgacacttcttggcaacaccaaggggagcgatagatcacactttttctcaaaaaatttcagaggatagctaaaataagtcgttcacattcctgggtttcagcctgctatggctcctggcttccttctatcagatggtgagaaaagcccacattttctttatctctgtatcccagcatctagcacagtgcctcaggtgtaataggtaggttttcagaattttcagaacgccctgctctgaataggtccagctctgagctgggtgttttggaagatacagaggcaaagtacatattcaagaaacttacacatgaactgtgaaggcctcagcttgaattctagttccatttatattcttcaaagcagcttccctccaggacctgagtcctgagtacttttcttcacagtctgggaagcaggttaaccttgtcagtgttggaatattgaaacacttccacagcagttggccaaggttggcttctctgagctctccagctccctcctccccagcctagcacctcagtctctcccctgaccacttcttacagctcagtggctaaaagggtagttctgaaagccagtgcctccaggaaggcacacccatgctaccgcaaccagtgggtgccttctatatgacttgttagatattttgatatcctctgcttcccaaaatggattccatgacagcaggctgacaagtaaagaaaaccaccatgtactgagtacttacctatacagtctcatctagtcttcaaaaccaccacaaatggaggtactcttattccacctgacaggtgaagttgagggagattaaaaaggttgttaaaggtaccccagagggtaggcaaatgagggttgaagcccaagtccatgtggctcccagtcttgaactctctcccctcccctcattgcctctcaagcggcaggcagcgttcctgtggtagggttgcaactagcttgcctggccataaataatgaaccttcttccctttgtggatggcattctatgttttaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttctgaaagttttctatacctgtctcctttctcagccacaaaataaatgttgggagaaatgatatataccactttcccagaaaaaaaaaaacttacacttgggacttggcaaattcctagtcacaatttttttcagcagtaacaggaaaccacttatcacatggagacctaatgtaataatagaaaaatactcataatagggagaaaccaagagaagttttgtttttgtttttttccaactgtgttcattagaacagcgtgttctaagtatttgaaactgaatgtttattccttgatactaaaagttcttctccaatcctatcactgatagtgtccaaattctcaccaaattgctcctaagcttcaaatcagaagcagaaactggcaggccatggaccttaattgtccctcaggtagattttgtttggtatgcagaatgtttttaaaatatgagtggttattgaaaatatgatgtttcacataaaacctcattctcggacccatctttgctcatggcaacagttagctggagctgagtagcagctgcctgattagatgactctcagtccccatggcaccctgctccatgttacctagagcaggcacttgattccttgctgggcagtatccaataggcatttgattttgcccactcctacactaagcgaatgtgtacaaagtgtaaatgcattaggaaaaacaaactacccgcatcttctgttaggcaggatctgtacaataataattatgagtttgcttatgtaatctcacctcacctggatgatcactaatactaattcatttattactaaccttctggcttccttctctcaatatgcttacaaagtctccagtcacctacaatgctggctttctcccactgagtttgctgtttgcaatttttccatgaagtttgaacttcataaggtaattcatggcattgaactggttcatgaaaagaacactagagtctgtcatttgctttggcttgaagtatggttggtaacacaaattttcacctgctcttctaccatttgaatttgtgtagagggtgtttgcagagcaatgcccgtaatgcttagagaatgttctcctaaaagacttgcggaatcactctgtccttggaagtttcatatattgtttgatatgaagtgttagatagaatttccaatattggagcatatcaaaaagtattaaaactaaaaaggaccagagaattcttagattggcccggaaaggccaataaagagttagaatgaaaactcattacttttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttggtattcttaagggaacttccacattatacatttgatgtattgaccagaatatgtaaaatatgcttataaatcagaaaaataaattgtttctcactaagtcaaaagaaaaaaagctcatgtttccaccacaaa

SEQ ID NO: 2 - RNA, 2394 nt, Homo sapiens
  source 1..2394: mol_type = mRNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 3 - RNA, 2394 nt, Homo sapiens
  source 1..2394: mol_type = mRNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcagataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 4 - RNA, 2394 nt, Homo sapiens
  source 1..2394: mol_type = mRNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggctttgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 5 - RNA, 2394 nt, Homo sapiens
  source 1..2394: mol_type = mRNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacaggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 6 - RNA, 4815 nt, Homo sapiens
  source 1..4815: mol_type = mRNA
ggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgaattggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccccgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttcttctgaaagttttctatacctgtctcctttctcagccacaaaataaatgttgggagaaatgatatataccactttcccagaaaaaaaaaacttacacttgggacttggcaaattcctagtcacaatttttttcagcagtaacaggaaaccacttatcacatggagacctaatgtaataatagaaaaatactcataatagggagaaaccaagagaagttttgtttttgtttttttccaactgtgttcattagaacagcgtgttctaagtatttgaaactgaatgtttattccttgatactaaaagatcttctccaatcctatcactgatagtgtccaaattctcaccaaattgctcctaagcttcaaatcagaagcagaaactggcaggccatggaccttaattgtccctcaggtagattttgtttggtatgcagaatgtttttaaaatatgagtggttattgaaaatatgatgtttcacataaaacctcattctcggacccatctttgctcatggcaacagttagctggagctgagtagcagctgcctgattagatgactctcagtccccatggcaccctgctccatgttacctagagcaggcacttgattccttgctgggcagtatccaataggcatttgattttgcccactcctacactaagcgaatgtgtacaaagtgtaaatgcattaggaaaaacaaactacccgcatcttctgttaggcaggatctgtacaataataattatgagtttgcttatgtaatctcacctcacctggatgatcactaataccaattcatttattactaaccttctggcttccttctctcaatatgcttacaaagtctccagtcacctacaatgctggctttctcccactgagtttgctgtttgcaatttttccatgaagtttgaacttcataaggtaattcatggcattgaactggttcatgaaaagaacactagagtctgtcatttgctttggcttgaagtatggttggtaacacaaattttcacctgctcttctaccatttgaatttgtgtagagggtgtttgcagagcaatgcccgtaatgcttagagaatgttctcctaaaagacttgcggaatcactctgtccttggaagtttcatatattgtttgatatgaagtgttagatagaatttccaatattggagcatatcaaaaagtattaaaactaaaaaggaccagagaattcttagattggcccggaaaggccaataaagagttagaatgaaaactcattacttttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttggtattcttaagggaacttccacattatacatttgatgtattgaccagaatatgtaaaatatgcttataaatcagaaaaataaattgtttctcactaagtc

SEQ ID NO: 7 - RNA, 5037 nt, Homo sapiens
  source 1..5037: mol_type = mRNA
aagcgcttcactgagcgctcgccgccgcccagcctctcctctcgcgcctcctagctcttcgcagagcaaccaggagccaggagtggtctagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcggcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaactgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttctgaaagttttctatacctgtctcctttctcagccacaaaataaatgttgggagaaatgatatataccactttcccagaaaaaaaaaaacttacacttgggacttggcaaattcctagtcacaatttttttcagcagtaacaggaaaccacttatcacatggagacctaatgtaataatagaaaaatactcataatagggagaaaccaagagaagttttgtttttgtttttttccaactgtgttcattagaacagcgtgttctaagtatttgaaactgaatgtttattccttgatactaaaagttcttctccaatcctatcactgatagtgtccaaattctcaccaaattgctcctaagcttcaaatcagaagcagaaactggcaggccatggaccttaattgtccctcaggtagattttgtttggtatgcagaatgtttttaaaatatgagtggttattgaaaatatgatgtttcacataaaacctcattctcggacccatctttgctcatggcaacagttagctggagctgagtagcagctgcctgattagatgactctcagtccccatggcaccctgctccatgttacctagagcaggcacttgattccttgctgggcagtatccaataggcatttgattttgcccactcctacactaagcgaatgtgtacaaagtgtaaatgcattaggaaaaacaaactacccgcatcttctgttaggcaggatctgtacaataataattatgagtttgcttatgtaatctcacctcacctggatgatcactaatactaattcatttattactaaccttctggcttccttctctcaatatgcttacaaagtctccagtcacctacaatgctggctttctcccactgagtttgctgtttgcaatttttccatgaagtttgaacttcataaggtaattcatggcattgaactggttcatgaaaagaacactagagtctgtcatttgctttggcttgaagtatggttggtaacacaaattttcacctgctcttctaccatttgaatttgtgtagagggtgtttgcagagcaatgcccgtaatgcttagagaatgttctcctaaaagacttgcggaatcactctgtccttggaagtttcatatattgtttgatatgaagtgttagatagaatttccaatattggagcatatcaaaaagtattaaaactaaaaaggaccagagaattcttagattggcccggaaaggccaataaagagttagaatgaaaactcattacttttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttggtattcttaagggaacttccacattatacatttgatgtattgaccagaat

SEQ ID NO: 8 - RNA, 3298 nt, Homo sapiens
  source 1..3298: mol_type = mRNA
gttgtcgactgtcaggaccgaagcgcttcactgagcgctcgccgccgcccagcctctcctctcgcgcctcctagctcttcgcagagcaaccaggagccaggagtggtctagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaactgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagcctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttctgaaagttttctatacctgtctcctttctcagccacaaaataaatgttgggagaaatgatatataccactttcccaaaaaaaaaaaaa

SEQ ID NO: 9 - RNA, 2233 nt, Homo sapiens
  source 1..2233: mol_type = mRNA
ggccagtcccactgcagggtcgagatgccgaactgactatggggaagggatctatttctttcttgttcttcagccaaattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaactgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacggaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtg

SEQ ID NO: 10 - RNA, 2450 nt, Homo sapiens
  source 1..2450: mol_type = mRNA
accaggagccaggagtggtccagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtc

SEQ ID NO: 11 - RNA, 2450 nt, Homo sapiens
  source 1..2450: mol_type = mRNA
accaggagccaggagtggtccagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccaacaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtc

SEQ ID NO: 12 - RNA, 2276 nt, Homo sapiens
  source 1..2276: mol_type = mRNA
gcgatcgccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatgtttaaac

SEQ ID NO: 13 - DNA, 2394 bp, Homo sapiens
  source 1..2394: mol_type = other DNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 14 - DNA, 2394 bp, Homo sapiens
  source 1..2394: mol_type = other DNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcagataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 15 - DNA, 2394 bp, Homo sapiens
  source 1..2394: mol_type = other DNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggctttgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 16 - DNA, 2394 bp, Homo sapiens
  source 1..2394: mol_type = other DNA
gttcgttgcaacaaattgatgagcaatgcttttttataatgccaactttgtacaaaaaagttggcaccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacaggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatttgccaactttcttgtacaaagttggcattataagaaagcattgcttatcaatttgttgcaacgaac

SEQ ID NO: 17 - DNA, 4815 bp, Homo sapiens
  source 1..4815: mol_type = other DNA
ggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgaattggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccccgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttcttctgaaagttttctatacctgtctcctttctcagccacaaaataaatgttgggagaaatgatatataccactttcccagaaaaaaaaaacttacacttgggacttggcaaattcctagtcacaatttttttcagcagtaacaggaaaccacttatcacatggagacctaatgtaataatagaaaaatactcataatagggagaaaccaagagaagttttgtttttgtttttttccaactgtgttcattagaacagcgtgttctaagtatttgaaactgaatgtttattccttgatactaaaagatcttctccaatcctatcactgatagtgtccaaattctcaccaaattgctcctaagcttcaaatcagaagcagaaactggcaggccatggaccttaattgtccctcaggtagattttgtttggtatgcagaatgtttttaaaatatgagtggttattgaaaatatgatgtttcacataaaacctcattctcggacccatctttgctcatggcaacagttagctggagctgagtagcagctgcctgattagatgactctcagtccccatggcaccctgctccatgttacctagagcaggcacttgattccttgctgggcagtatccaataggcatttgattttgcccactcctacactaagcgaatgtgtacaaagtgtaaatgcattaggaaaaacaaactacccgcatcttctgttaggcaggatctgtacaataataattatgagtttgcttatgtaatctcacctcacctggatgatcactaataccaattcatttattactaaccttctggcttccttctctcaatatgcttacaaagtctccagtcacctacaatgctggctttctcccactgagtttgctgtttgcaatttttccatgaagtttgaacttcataaggtaattcatggcattgaactggttcatgaaaagaacactagagtctgtcatttgctttggcttgaagtatggttggtaacacaaattttcacctgctcttctaccatttgaatttgtgtagagggtgtttgcagagcaatgcccgtaatgcttagagaatgttctcctaaaagacttgcggaatcactctgtccttggaagtttcatatattgtttgatatgaagtgttagatagaatttccaatattggagcatatcaaaaagtattaaaactaaaaaggaccagagaattcttagattggcccggaaaggccaataaagagttagaatgaaaactcattacttttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttggtattcttaagggaacttccacattatacatttgatgtattgaccagaatatgtaaaatatgcttataaatcagaaaaataaattgtttctcactaagtc

SEQ ID NO: 18 - DNA, 5037 bp, Homo sapiens
  source 1..5037: mol_type = other DNA
aagcgcttcactgagcgctcgccgccgcccagcctctcctctcgcgcctcctagctcttcgcagagcaaccaggagccaggagtggtctagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcggcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaactgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttctgaaagttttctatacctgtctcctttctcagccacaaaataaatgttgggagaaatgatatataccactttcccagaaaaaaaaaaacttacacttgggacttggcaaattcctagtcacaatttttttcagcagtaacaggaaaccacttatcacatggagacctaatgtaataatagaaaaatactcataatagggagaaaccaagagaagttttgtttttgtttttttccaactgtgttcattagaacagcgtgttctaagtatttgaaactgaatgtttattccttgatactaaaagttcttctccaatcctatcactgatagtgtccaaattctcaccaaattgctcctaagcttcaaatcagaagcagaaactggcaggccatggaccttaattgtccctcaggtagattttgtttggtatgcagaatgtttttaaaatatgagtggttattgaaaatatgatgtttcacataaaacctcattctcggacccatctttgctcatggcaacagttagctggagctgagtagcagctgcctgattagatgactctcagtccccatggcaccctgctccatgttacctagagcaggcacttgattccttgctgggcagtatccaataggcatttgattttgcccactcctacactaagcgaatgtgtacaaagtgtaaatgcattaggaaaaacaaactacccgcatcttctgttaggcaggatctgtacaataataattatgagtttgcttatgtaatctcacctcacctggatgatcactaatactaattcatttattactaaccttctggcttccttctctcaatatgcttacaaagtctccagtcacctacaatgctggctttctcccactgagtttgctgtttgcaatttttccatgaagtttgaacttcataaggtaattcatggcattgaactggttcatgaaaagaacactagagtctgtcatttgctttggcttgaagtatggttggtaacacaaattttcacctgctcttctaccatttgaatttgtgtagagggtgtttgcagagcaatgcccgtaatgcttagagaatgttctcctaaaagacttgcggaatcactctgtccttggaagtttcatatattgtttgatatgaagtgttagatagaatttccaatattggagcatatcaaaaagtattaaaactaaaaaggaccagagaattcttagattggcccggaaaggccaataaagagttagaatgaaaactcattacttttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttggtattcttaagggaacttccacattatacatttgatgtattgaccagaat

SEQ ID NO: 19 - DNA, 3298 bp, Homo sapiens
  source 1..3298: mol_type = other DNA
gttgtcgactgtcaggaccgaagcgcttcactgagcgctcgccgccgcccagcctctcctctcgcgcctcctagctcttcgcagagcaaccaggagccaggagtggtctagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaactgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtggttgttttgtcatgaattcttatgcttataatatcctttgtggcaccttttctttttctccctaaactgtacatgtgaaggggatgagctcaagcaggaagttcaacttccagaattgatcataggtatttcaaaacacatctttcctgtctgcacaagtgaagtgttttgttctttctggagtcacagttgacaaaaagctcttacactacattagaacactgcattagagcccatttcaattctcaaaagaaaaggcaaaacctgggatatcaattaatttgaaaacataatctgcaaagaatgagaaggagtcagaaactgtttctgtagcttgttccctgtcttgtccatgtggttcttcaaattttgatgccaagaaagtatttggtaggcctaatgaaggagttcactgtaagactcattccctagatctttctattccaaagtgccactcattcctgtagtcaaaatctggtcatgttggtcaaaagcctggattatttagatctagaaacagatcttgaaatctgaatgctctggtttgagcaattttcgaacattctttgcctggtgcactgtgtctgtggtgccagaggcgtccgtggatccagaggtggttatgactcgtgctgcatgcctggtctttcctctgtttctccttctgaaagttttctatacctgtctcctttctcagccacaaaataaatgttgggagaaatgatatataccactttcccaaaaaaaaaaaaa

SEQ ID NO: 20 - DNA, 2233 bp, Homo sapiens
  source 1..2233: mol_type = other DNA
ggccagtcccactgcagggtcgagatgccgaactgactatggggaagggatctatttctttcttgttcttcagccaaattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaactgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacggaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtctgaagtg

SEQ ID NO: 21 - DNA, 2450 bp, Homo sapiens
  source 1..2450: mol_type = other DNA
accaggagccaggagtggtccagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtc

SEQ ID NO: 22 - DNA, 2450 bp, Homo sapiens
  source 1..2450: mol_type = other DNA
accaggagccaggagtggtccagagcccgagggtgggaagggggagtctgtctggcttttctcctatcttgcttctttttcctcttcccttcccactcttgttcaagcgagtgtgtgagctatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccaacaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttccgaggccatgctgcgactcctgcaaagtgctttcagtaaaaactcaccgccaaagcaatcaccaaagaagtccccaactgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaattaaaataagtgtgtggtcccaagttggaaatattcatgcttcttccttaccctgcgattttgcctgtgtc

SEQ ID NO: 23 - DNA, 2276 bp, Homo sapiens
  source 1..2276: mol_type = other DNA
gcgatcgccatggagcgaagagcctggagtctgcagtgcactgctttcgtcctcttttgcgcttggtgtgcactgaacagtgcaaaagcgaaaaggcaatttgtcaatgaatgggcagcggagatccccgggggcccggaagcagcctcggccatcgccgaggagctgggctatgaccttttgggtcagattggttcacttgaaaatcactacttattcaaacataaaaaccaccccagaaggtctcgaaggagtgcctttcatatcactaagagattatctgatgatgatcgtgtgatatgggctgaacaacagtatgaaaaagaaagaagtaaacgttcagctctaagggactcagcactaaatctcttcaatgatcccatgtggaatcagcaatggtacttgcaagataccaggatgacggcagccctgcccaagctggaccttcatgtgatacctgtttggcaaaaaggcattacgggcaaaggagttgttatcaccgtactggatgatggtttggagtggaatcacacggacatttatgccaactatgatccagaggctagctatgattttaatgataatgaccatgatccatttccccgatatgatcccacaaacgagaacaaacacgggaccagatgtgcaggagaaattgccatgcaagcaaataatcacaaatgcggggttggagttgcatacaattccaaagttggaggcataagaatgctggatggcattgtgacggatgctattgaggccagttcaattggattcaatcctggacacgtggatatttacagtgcaagctggggccctaatgatgatgggaaaactgtggaggggcctggccggctagcccagaaggcttttgaatatggtgtcaaacaggggagacaggggaaggggtccatcttcgtctgggcttcgggaaacggggggcgtcagggagataattgtgactgtgatggctacacagacagcatctacaccatctccatcagcagtgcctcccagcaaggcctatccccctggtacgctgagaagtgctcctccacactggccacctcttacagcagcggagattacaccgaccagagaatcacgagcgctgacctgcacaatgactgcacggagacgcacacaggcacctcggcctctgcacctctggctgctggcatcttcgctctggccctggaagcaaacccaaatctcacctggcgagatatgcagcacctggttgtctggacctctgagtatgacccgctggccaataaccctggatggaaaaagaatggagcaggcttgatggtgaatagtcgatttggatttggcttgctaaatgccaaagctctggtggatttagctgaccccaggacctggaggagcgtgcctgagaagaaagagtgtgttgtaaaggacaatgactttgagcccagagccctgaaagctaatggagaagttatcattgaaattccaacaagagcttgtgaaggacaagaaaatgctatcaagtccctggagcatgtacaatttgaagcaacaattgaatattcccgaagaggagaccttcatgtcacacttacttctgctgctggaactagcactgtgctcttggctgaaagagaacgggatacatctcctaatggctttaagaattgggacttcatgtctgttcacacatggggagagaaccctataggtacttggactttgagaattacagacatgtctggaagaattcaaaatgaaggaagaattgtgaactggaagctgattttgcacgggacctcttctcagccagagcatatgaagcagcctcgtgtgtacacgtcctacaacactgttcagaatgacagaagaggggtggagaagatggtggatccaggggaggagcagcccacacaagagaaccctaaggagaacaccctggtgtccaaaagccccagcagcagcagcgtagggggccggagggatgagttggaggagggagccccttcccaggccatgctgcgactcctgcaaagtgctttcagtaaaaactcaccgccaaagcaatcaccaaagaagtccccaagtgcaaagctcaacatcccttatgaaaacttctacgaagccctggaaaagctgaacaaaccttcccagcttaaagactctgaagacagtctgtataatgactatgttgatgttttttataacactaaaccttacaagcacagagacgaccggctgcttcaagctctggtggacattctgaatgaggaaaatgtttaaac

SEQ ID NO: 24 - protein, 753 aa, Homo sapiens [sequence omitted]

SEQ ID NO: 25 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQADNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSEAMLRLLQSAFSKNSPPKQSPKKSPTAKLNIPYENFYEALEKLNKPSQLKDSEDSLYNDYVDVFYNTKPYKHRDDRLLQALVDILNEEN

SEQ ID NO: 26 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWALGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSEAMLRLLQSAFSKNSPPKQSPKKSPTAKLNIPYENFYEALEKLNKPSQLKDSEDSLYNDYVDVFYNTKPYKHRDDRLLQALVDILNEEN

SEQ ID NO: 27 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHRTSSQPEHMKQPRVYTSYNTVQNDRRGVEKMVDPGEEQPTQENPKENTLVSKSPSSSSVGGRRDELEEGAPSEAMLRLLQSAFSKNSPPKQSPKKSPTAKLNIPYENFYEALEKLNKPSQLKDSEDSLYNDYVDVFYNTKPYKHRDDRLLQALVDILNEEN

SEQ ID NO: 28 - protein, 724 aa, Homo sapiens
QFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRIGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSEAMLRLLQSAFSKNSPPKQSPKKSPTAKLNIPYENFYEALEKLNKPSQLKDSEDSLYNDYVDVFYNTKPYKHRDDRLLQALVDILNEEN

SEQ ID NO: 29 - protein, 753 aa, Homo sapiens [sequence omitted]

SEQ ID NO: 30 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 31 - protein, 706 aa, Homo sapiens [sequence omitted]

SEQ ID NO: 32 - protein, 4 aa, synthetic construct
  REGION 1..4: note = MC4R agonist
HFRW